(19) United States
(12) Patent Application Publication
(10) Pub. No.: US 2026/0116956 A1
(43) Pub. Date: Apr. 30, 2026

(54) ANTIBODY BASED TREATMENT TO REDUCE GUT BACTERIAL GLYCAN AND ASSOCIATED BACTERIA

(71) Applicant: NEW YORK UNIVERSITY, New York, NY (US)

(72) Inventors: Gregg J. SILVERMAN, New York, NY (US); Doua AZZOUZ, New York, NY (US)

(73) Assignee: NEW YORK UNIVERSITY, New York, NY (US)

(21) Appl. No.: 18/876,167

(22) PCT Filed: Jun. 21, 2023
(86) PCT No.: PCT/US2023/068808

§ 371 Date: Dec. 17, 2024

Related U.S. Application Data
(60) Provisional application No. 63/354,159, filed on Jun. 21, 2022.

(51) Int. Cl. C07K 16/1267; A61K 39/00; A61K 39/40; A61K 47/68; A61P 13/12; C12N 1/20; C12N 15/86

(52) U.S. Cl. CPC C07K 16/1267; A61K 39/40; A61K 47/6835; A61P 13/12; C12N 1/20; C12N 15/86; A61K 2039/53; A61K 2039/542; C07K 2317/14; C07K 2317/565

(57) ABSTRACT

This application provides isolated antibodies, or antigen-binding fragments thereof, that specifically recognize one or more gut bacterial lipoglycans, and polynucleotides and vectors that encode for such antibodies or antigen-binding fragments. This application further provides methods of producing the antibodies or antigen-binding fragments and using the antibodies or antigen-binding fragments for treatment of diseases.

### CROSS-REFERENCE TO RELATED APPLICATIONS

This patent application claims the benefit of U.S. Provisional Application No. 63/354,159, filed Jun. 21, 2022, the disclosure of which is incorporated by reference herein in its entirety for all purposes.

### STATEMENT REGARDING FEDERALLY SPONSORED RESEARCH

This invention was made with government support under AR42455 and AR070591 awarded by National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Jun. 20, 2023, is named 243735_000278_SL.xml and is 24,323 bytes in size.

### FIELD OF THE INVENTION

This application relates to isolated antibodies, or antigen-binding fragments thereof, that specifically recognize one or more gut bacterial lipoglycans, and polynucleotides and vectors that encode for such antibodies or antigen-binding fragments. This application further relates to methods of producing the antibodies or antigen-binding fragments and using the antibodies or antigen-binding fragments for treatment of diseases.

### BACKGROUND

The microbiome in the human gut is the largest collection of bacteria in the body, and the metabolic and biochemical pathways affect nutrition and immune setpoints. Barrier function, which delimits gut contents, cellular and microbial products is maintained to a large degree by tight junctions between intestinal epithelial cells, although imbalances or dysbiosis in the representation of certain bacteria or the local production of certain toxins can result in increased gut permeability, with entrance of bacterial components into the body itself to affect the systemic immune system. In murine model systems, it has been shown that gut colonization of mice with certain strains of Ruminococcus gnavus, a human commensal anerobic gram-positive spore-forming bacteria of the Lachnospiraceae family, and especially the strains isolated from fecal samples of Lupus patients, can induce increased gut permeability that is associated with adverse health effects.

B lymphocytes can develop into end-differentiated plasma cells that are high-level producers of antibodies. Antibodies of the IgA isotype are transported across the gut epithelial layer by a facilitated transport process. Some of these locally produced antibodies are induced by immune recognition and clonal selection of B cell clones specific for the bacteria and bacteria products present in the gut or at other sites. Encounter of such an antibody with its cognate antigen, either as a secreted product or on the surface of a B cells can interfere with the biologic functions of the bacterial product, with examples of cholera toxin and adhesion factors that contribute to bacterial pathogenic pathways. Formation of these complexes may therefore interfere with bacterial pathogenic pathways, including those with the host epithelial cells, which can have consequences for the host-bacteria relationship. Some may have benefits for the host. Formation of complexes of host antibody with secreted or bacteria associated factors can hasten their removal from the body, and can decrease the representation within the bowel of such bacteria by hastening their excretion.

### SUMMARY OF THE INVENTION

As specified in the Background section above, there is a great need in the art for antibodies that target pathogenic bacterial antigens. The present application addresses these and other needs.

In one aspect, provided herein is an isolated antibody, or antigen-binding fragment thereof, that specifically recognizes a lipoglycan-containing antigen associated with Ruminococcus gnavus strain CC55_001C/HM-1056, S107-86, S47-18, and/or S107-48.

In certain embodiments, the lipoglycan-containing antigen comprises a lipoglycan which has a molecular weight between about 20,000 and about 30,000 Daltons. In certain embodiments, the lipoglycan-containing antigen comprises a lipoglycan selected from those listed in Table 7.

In certain embodiments, the lipoglycan-containing antigen comprises a lipoglycan comprising three fatty acids with acyl chain composition of 47:0, one glycerol, eight hexoses, five N-acetyl-hexosamines, and three hexuronic acids (tri-acyl lipoglycan 47:0 with a mono-isotopic mass of about 3632).

In certain embodiments, the lipoglycan-containing antigen comprises a lipoglycan comprising two fatty acids with acyl chain composition of 31:0, one glycerol, eight hexoses, five N-acetyl-hexosamines, and three hexuronic acids (di-acyl lipoglycan 31:0 with a mono-isotopic mass of about 3394).

In certain embodiments, the lipoglycan-containing antigen comprises a lipoglycan comprising one fatty acid with acyl chain composition of 16:0, one glycerol, eight hexoses, five N-acetyl-hexosamines, and three hexuronic acids (mono-acyl lipoglycan 16:0 with a mono-isotopic mass of about 3170).

In certain embodiments, the lipoglycan-containing antigen comprises a lipoglycan comprising one margaric acid with acyl chain composition of 17:0, one glycerol, eight hexoses, five N-acetyl-hexosamines, and three hexuronic acids (mono-acyl lipoglycan 17:0 with a mono-isotopic mass of about 3184).

In certain embodiments, the lipoglycan-containing antigen is the same as the lipoglycan-containing antigen obtained using a method selected from:
(i) Method 1 comprising the steps:
   a) culturing Ruminococcus gnavus strain CC55_001C/HIM-1056, S107-86, S47-18, or S107-48, or any of other strains of Ruminococcus gnavus that produces a lipoglycan identified by structural analysis with above-described features, and/or based on immunoreactivity with lipoglycan specific monoclonal antibodies, at 37° C. under anaerobic conditions for 2-7 days, and
   b) producing bacterial extract in the presence of a lysozyme, Serratia marcescens endonuclease, Proteinase K, and a detergent under non-denaturing conditions;
(ii) Method 2 comprising the steps:
   a) culturing Ruminococcus gnavus strain CC55_001C/HIM-1056, S107-86, S47-18, or S107-48, or another strain of Ruminococcus gnavus that produces a lipoglycan as described herein at 37° C. under anaerobic conditions (75% N₂, 20% CO₂, and 5% H₂) for 2-7 days,
   b) pelleting bacteria by centrifugation,
   c) producing a bacterial extract in a protein extraction buffer in the presence of lysozyme, Serratia marcescens endonuclease, and a detergent under non-denaturing conditions,
   d) treating the extract obtained in step (c) with Proteinase K,
   e) incubating the treated extract obtained in step (d) at 55° C. for about 10 minutes,
   f) removing cell debris by centrifugation, and
   g) using the supernatant as an antigen preparation; or
(iii) Method 3 comprising the steps:
   a) disrupting Ruminococcus gnavus strain CC55_001C/HM-1056, S107-86, S47-18, or S107-48 cells or another strain of Ruminococcus gnavus that produces a lipoglycan as described herein with a French press,
   b) ultracentrifugating to obtain a precipitate and produce an ultracentrifugation supernatant,
   c) subjecting the ultracentrifugation supernatant obtained in step (b) to butanol-water extraction and isolating therefrom an aqueous phase,
   d) applying the aqueous phase from step (c) to a hydrophobic interaction chromatography matrix, and
   e) isolating lipoglycan-containing fractions.

In certain embodiments, the isolated antibody, or antigen-binding fragment thereof, comprises a heavy chain complementarity determining region 1 (CDR1), a heavy chain CDR2, a heavy chain CDR3 of a heavy chain variable region (VH) comprising an amino acid sequence of SEQ ID NO: 4 or 13; and/or a light chain CDR1, a light chain CDR1, a light chain CDR2, and a light chain CDR3 of a light chain variable region (VL) comprising an amino acid sequence of SEQ ID NO: 9 or 15.

In certain embodiments, the isolated antibody, or antigen-binding fragment thereof, comprises a heavy chain CDR1, a heavy chain CDR2 and a heavy chain CDR3 of a VH comprising an amino acid sequence of SEQ ID NO: 4, and a light chain CDR1, a light chain CDR2 and a light chain CDR3 of a VL comprising an amino acid sequence of SEQ ID NO: 9; or a heavy chain CDR1, a heavy chain CDR2 and a heavy chain CDR3 of a VH comprising an amino acid sequence of SEQ ID NO: 13, and a light chain CDR1, a light chain CDR2 and a light chain CDR3 of a VL comprising an amino acid sequence of SEQ ID NO: 15.

In certain embodiments, the isolated antibody, or antigen-binding fragment thereof, comprises a heavy chain CDR1 comprising the amino acid sequence of SEQ ID NO: 1, a heavy chain CDR2 comprising the amino acid sequence of SEQ ID NO: 2 or 11, and a heavy chain CDR3 comprising the amino acid sequence of SEQ ID NO: 3 or 12.

In certain embodiments, the isolated antibody, or antigen-binding fragment thereof, comprises a light chain CDR1 comprising the amino acid sequence of SEQ ID NO: 6, a light chain CDR2 comprising the amino acid sequence of SEQ ID NO: 7, and a light chain CDR3 comprising the amino acid sequence of SEQ ID NO: 8.

In certain embodiments, the isolated antibody, or antigen-binding fragment thereof, comprises the heavy chain CDR1 of SEQ ID NO: 1, the heavy chain CDR2 of SEQ ID NO: 2, the heavy chain CDR3 of SEQ ID NO: 3, the light chain CDR1 of SEQ ID NO: 6, the light chain CDR2 of SEQ ID NO: 7 and the light chain CDR3 of SEQ ID NO: 8; or the heavy chain CDR1 of SEQ ID NO: 1, the heavy chain CDR2 of SEQ ID NO: 11, the heavy chain CDR3 of SEQ ID NO: 12, the light chain CDR1 of SEQ ID NO: 6, the light chain CDR2 of SEQ ID NO: 7 and the light chain CDR3 of SEQ ID NO: 8.

In certain embodiments, the isolated antibody, or antigen-binding fragment thereof, comprises a heavy chain CDR1 comprising the amino acid sequence of SEQ ID NO: 21, a heavy chain CDR2 comprising the amino acid sequence of SEQ ID NO: 22, and a heavy chain CDR3 comprising the amino acid sequence of SEQ ID NO: 3; or a heavy chain CDR1 comprising the amino acid sequence of SEQ ID NO: 21, a heavy chain CDR2 comprising the amino acid sequence of SEQ ID NO: 25, and a heavy chain CDR3 comprising the amino acid sequence of SEQ ID NO: 12.

In certain embodiments, the isolated antibody, or antigen-binding fragment thereof, comprises a light chain CDR1 comprising the amino acid sequence of SEQ ID NO: 24, a light chain CDR2 comprising the amino acid sequence of KAS, and a light chain CDR3 comprising the amino acid sequence of SEQ ID NO: 8.

In certain embodiments, the isolated antibody, or antigen-binding fragment thereof, comprises the heavy chain CDR1 of SEQ ID NO: 21, the heavy chain CDR2 of SEQ ID NO: 22, the heavy chain CDR3 of SEQ ID NO: 3, the light chain CDR1 of SEQ ID NO: 24, the light chain CDR2 of KAS and the light chain CDR3 of SEQ ID NO: 8; or the heavy chain CDR1 of SEQ ID NO: 21, the heavy chain CDR2 of SEQ ID NO: 25, the heavy chain CDR3 of SEQ ID NO: 12, the light chain CDR1 of SEQ ID NO: 24, the light chain CDR2 of KAS, and the light chain CDR3 of SEQ ID NO: 8.

In certain embodiments, the isolated antibody, or antigen-binding fragment thereof, comprises a VH comprising an amino acid sequence of SEQ ID NO: 4 or 13; and/or a VL comprising an amino acid sequence of SEQ ID NO: 9 or 15.

In certain embodiments, the isolated antibody, or antigen-binding fragment thereof, comprises a VH comprising an amino acid sequence of SEQ ID NO: 4 and a VL comprising an amino acid sequence of SEQ ID NO: 9; or a VH comprising an amino acid sequence of SEQ ID NO: 13 and a VL comprising an amino acid sequence of SEQ ID NO: 15.

In certain embodiments, the antibody or antigen-binding fragment is recombinant. In certain embodiments, the antibody or antigen-binding fragment is a human antibody, a humanized antibody, a chimeric antibody, a murine antibody, a monoclonal antibody, a single chain antibody, a Fab, a Fab′, a F(ab′)2, a Fv, or a scFv or a nanobody.

In certain embodiments, the antibody or antigen-binding fragment is an IgG antibody. In certain embodiments, the antibody or antigen-binding fragment is of IgG1, IgG2, IgG3, or IgG4 subclass. In certain embodiments, the antibody or antigen-binding fragment is of IgG1 subclass. In certain embodiments, the antibody or antigen-binding fragment is of IgG2 subclass.

In another aspect, provided herein is an isolated polynucleotide encoding the isolated antibody or antigen-binding fragment of the antibodies disclosed herein.

In certain embodiments, the isolated polynucleotide, comprises a VH-encoding nucleotide sequence of SEQ ID NO: 5 or 14; and/or a VL-encoding nucleotide sequence of SEQ ID NO: 10 or 16.

In certain embodiments, the isolated polynucleotide, comprises a VH-encoding nucleotide sequence of SEQ ID NO: 5 and a VL-encoding nucleotide sequence of SEQ ID NO: 10; or a VH-encoding nucleotide sequence of SEQ ID NO: 14 and a VL-encoding nucleotide sequence of SEQ ID NO: 16.

In certain embodiments, the isolated polynucleotide is a DNA molecule or a derivative thereof. In certain embodiments, the isolated polynucleotide is an RNA molecule or a derivative thereof. In certain embodiments, the isolated polynucleotide is an mRNA.

In another aspect, provided herein is a vector comprising the polynucleotide disclosed herein. In certain embodiments, the sequence encoding the isolated antibody or antigen-binding fragment is operably linked to a promoter, wherein the promoter mediates expression of the isolated antibody or antigen-binding fragment. In certain embodiments, the vector is a viral vector. In certain embodiments, vector has tropism to the gastrointestinal tract.

In another aspect, provided herein is a recombinant cell comprising the polynucleotide disclosed herein or a vector disclosed herein. In certain embodiments, the cell is a hybridoma.

In certain embodiments, the antibody or antigen-binding fragment is recombinantly produced.

In another aspect, provided herein is an antibody-drug conjugate comprising an isolated antibody or antigen-binding fragment disclosed herein conjugated to a second moiety. In certain embodiments, the second moiety is a bacterial toxin or antibiotic.

In another aspect, provided herein is a pharmaceutical composition comprising an isolated antibody or antigen-binding fragment disclosed herein, or the polynucleotide disclosed herein, or the vector disclosed herein, or the antibody-drug conjugate disclosed herein and a pharmaceutically acceptable excipient or carrier.

In certain embodiments, the pharmaceutical composition is milk, yogurt, infant formula, or other dairy product.

In certain embodiments, the pharmaceutical composition is formulated such that it is released primarily in the gastrointestinal tract. In certain embodiments, the pharmaceutical composition is formulated such that it is released primarily in the small intestine. In certain embodiments, the pharmaceutical composition is formulated such that it is released primarily in the ileum.

In another aspect, provided herein is a vaccine composition comprising the polynucleotide disclosed herein or the vector disclosed herein. In certain embodiments, the polynucleotide is an mRNA.

In another aspect, provided herein is a kit comprising (i) an isolated antibody or antigen-binding fragment disclosed herein, or the polynucleotide disclosed herein, or the vector disclosed herein, or the antibody-drug conjugate disclosed herein and (ii) packaging and/or instructions for the same.

In another aspect, provided herein is a method of producing an isolated antibody or antigen-binding fragment disclosed herein, wherein said method comprises culturing the recombinant cell disclosed herein, and isolating the antibody or antigen-binding fragment.

In another aspect, provided herein is a method of producing an isolated antibody or antigen-binding fragment disclosed herein, wherein said method comprises isolating said antibody or antigen-binding fragment from an animal immunized with a lipoglycan-containing antigen associated with Ruminococcus gnavus strain CC55_001C/HM-1056, S107-86, S47-18, and/or S107-48 or a Ruminococcus gnavus strain that produces a lipoglycan that is structurally or functionally equivalent to Ruminococcus gnavus strain CC55_001C/HM-1056, S107-86, S47-18, and/or S107-48, or colonized with Ruminococcus gnavus strain CC55_001C/HM-1056, S107-86, S47-18, and/or S107-48 or a Ruminococcus gnavus strain that produces a lipoglycan that is structurally or functionally equivalent to Ruminococcus gnavus strain CC55_001C/HM-1056, S107-86, S47-18, and/or S107-48 in the gastrointestinal tract, or genetically modified to produce said antibody or antigen-binding fragment.

In certain embodiments, the animal is genetically modified with a polynucleotide disclosed herein or a vector disclosed herein. In certain embodiments, the animal is a dairy animal and said antibody or antigen-binding fragment is isolated from milk produced by the dairy animal. In certain embodiments, the dairy animal is a goat, a cow, a buffalo, a sheep, or a camel.

In another aspect, provided herein is a method of reducing the amount of a lipoglycan-containing antigen and/or a bacterial strain associated with the lipoglycan-containing antigen in a subject in need thereof, comprising administering to the subject an effective amount of an isolated antibody or antigen-binding fragment disclosed herein, or a disclosed herein, or a vector disclosed herein, or an antibody-drug conjugate disclosed herein, or a pharmaceutical composition disclosed herein.

In another aspect, provided herein is a method of treating or preventing a disease or disorder caused by a lipoglycan-containing antigen and/or a bacterial strain associated with the lipoglycan-containing antigen in a subject in need thereof, comprising administering to the subject an effective amount of an isolated antibody or antigen-binding fragment disclosed herein, or a polynucleotide disclosed herein, or a vector disclosed herein, or an antibody-drug conjugate disclosed herein, or a pharmaceutical composition disclosed herein, or a vaccine disclosed herein.

In certain embodiments, the lipoglycan-containing antigen of the methods comprises a lipoglycan which has a molecular weight between about 20,000 and about 30,000 Daltons. In certain embodiments, the lipoglycan-containing antigen of the methods comprises a lipoglycan selected from those listed in Table 7.

In certain embodiments, the lipoglycan-containing antigen of the methods comprises a lipoglycan comprising three fatty acids with acyl chain composition of 47:0, one glycerol, eight hexoses, five N-acetyl-hexosamines, and three hexuronic acids (tri-acyl lipoglycan 47:0 with a mono-isotopic mass of about 3632).

In certain embodiments, the lipoglycan-containing antigen of the methods comprises a lipoglycan comprising two fatty acids with acyl chain composition of 31:0, one glycerol, eight hexoses, five N-acetyl-hexosamines, and three hexuronic acids (di-acyl lipoglycan 31:0 with a mono-isotopic mass of about 3394).

In certain embodiments, the lipoglycan-containing antigen of the methods comprises a lipoglycan comprising one fatty acid with acyl chain composition of 16:0, one glycerol, eight hexoses, five N-acetyl-hexosamines, and three hexuronic acids (mono-acyl lipoglycan 16:0 with a mono-isotopic mass of about 3170).

In certain embodiments, the lipoglycan-containing antigen of the methods comprises a lipoglycan comprising one margaric acid with acyl chain composition of 17:0, one glycerol, eight hexoses, five N-acetyl-hexosamines, and three hexuronic acids (mono-acyl lipoglycan 17:0 with a mono-isotopic mass of about 3184).

In certain embodiments, the lipoglycan-containing antigen of the methods is the same as the lipoglycan-containing antigen obtained using a method selected from:
(i) Method 1 comprising the steps:
   a) culturing Ruminococcus gnavus strain CC55_001C/HM-1056, S107-86, S47-18, or S107-48 or any of other strains of Ruminococcus gnavus that produces a lipoglycan identified by structural analysis with above-described features, and/or based on immunoreactivity with lipoglycan specific monoclonal antibodies, at 37° C. under anaerobic conditions for 2-7 days, and
   b) producing bacterial extract in the presence of a lysozyme, Serratia marcescens endonuclease, Proteinase K, and a detergent under non-denaturing conditions;
(ii) Method 2 comprising the steps:
   a) culturing Ruminococcus gnavus strain CC55_001C/HM-1056, S107-86, 547-18, or S107-48 or another strain of Ruminococcus gnavus that produces a lipoglycan as described herein, in rich nutrient media at 37° C. under anaerobic conditions (75% N₂, 20% CO₂, and 5% H₂) for 2-7 days,
   b) pelleting bacteria by centrifugation,
   c) producing a bacterial extract in a protein extraction buffer in the presence of lysozyme, Serratia marcescens endonuclease, and a detergent under non-denaturing conditions,
   d) treating the extract obtained in step (c) with Proteinase K,
   e) incubating the treated extract obtained in step (d) at 55° C. for about 10 minutes,
   f) removing cell debris by centrifugation, and
   g) using the supernatant as an antigen preparation; or
(iii) Method 3 comprising the steps:
   a) disrupting Ruminococcus gnavus strain CC55_001C/HM-1056, S107-86, 547-18, or S107-48 cells or another strain of Ruminococcus gnavus that produces a lipoglycan as described herein, with a French press,
   b) ultracentrifugating to obtain a precipitate and produce an ultracentrifugation supernatant,
   c) subjecting the ultracentrifugation supernatant obtained in step (b) to butanol-water extraction and isolating therefrom an aqueous phase,
   d) applying the aqueous phase from step (c) to a hydrophobic interaction chromatography matrix, and
   e) isolating lipoglycan-containing fractions.

In certain embodiments, the bacterial strain is a strain of Ruminococcus gnavus. In certain embodiments, the lipoglycan-associated bacterial strain is Ruminococcus gnavus strain CC55_001C/HM-1056, S107-86, S47-18, and/or S107-48.

In certain embodiments, the methods relate to the disease or disorder is systemic lupus erythematosus (SLE), lupus nephritis, hidradenitis suppurativa, inflammatory bowel disease (IBD), incomplete lupus (ILE), undifferentiated connective tissue disease (UCTD), complications of SLE, IgA nephropathy, Henoch Schonlein Purpura (HSP), and other types of glomerulonephritis.

In certain embodiments, the lupus nephritis is proliferative lupus nephritis, membranous lupus nephritis, membranoproliferative lupus nephritis, or mesangial glomerulonephritis.

In certain embodiments, the isolated antibody or antigen-binding fragment, or the polynucleotide, or the vector, or the pharmaceutical composition disclosed herein is administered by a route selected from oral, nasal, rectal, mucosal, sublingual, and via naso/oro-gastric gavage.

In certain embodiments, the subject is a human.

### BRIEF DESCRIPTION OF THE DRAWINGS

FIGS. 1A-1C shows the immunoreactivity and binding specificity of the 36.2.2 monoclonal antibody (mAb) for the lipoglycan-expressing strains of Blautia (Ruminococcus) gnavus (RG). FIG. 1A shows a Coomassie stain for one of duplicate gels loaded equally with the whole extracts of a R. gnavus reference strain ATCC 29149 (RG1) that lacks the lipoglycan (LG, the two lupus RG strains (S107-48, S47-18) expressing LG, the reference strain CC55_001C (RG2) and LG purified from this strain (RG2-LG). FIG. 1B shows an Immunoblot with the mAb 36.2.2 using the other duplicate gel as in (B), the blot is showing the reactivity only to the LG band in the two RG lupus strains (S107-48, S47-18) and the reference strain (RG2) and its purified LG component. FIG. 1C shows immunoreactivity by ELISA reflected as ODs that reflects binding reactivity of the mAb 36.2.2 to all extracts and to both purified LGs from RG2 and the lupus strain S47-18. LPS from a Pseudomonas species.

FIGS. 2A-2D demonstrate diverse RG strains isolated from human donors that were then used to gut colonize germ-free (GF) mice and are passed to their litters. Abundance of RG deoxyribonucleic acid (DNA) in germ-free (GF) C57BL/6 mice breeding pairs, monocolonized by oral gavage with in RG1, RG2 and S107-48 RG strains. RG species-specific 16S rDNA genomic levels were quantified by genomic quantitative polymerase chain reaction (qPCR) analysis of fecal pellets, as shown. Inset values indicate normalized quantity of RG 16S rDNA (ribosomal RNA gene) with control mice, before (left) and weeks after (right) gavage with each of the RG stains, as indicated (FIG. 2A). Abundance of RG-specific genomic DNA in 4-week-old litters of the RG1, RG2 and S107-48-monocolonized GF breeding pairs. RG species-specific 16S rDNA levels were quantified by genomic qPCR analysis of fecal pellets, as shown (FIG. 2B). Abundance of RG DNA in fecal pellets of 12-14-week-old litters of the individual RG1, RG2 and S107-48 strains monocolonized GF breeding pairs (FIG. 2C). Abundance of RG DNA in cecal pellets of 12-14-week-old litters of RG1, RG2 and S107-48-monocolonized GF breeding pairs. Note that over time there was increased variation in the level of RG strain persistence as measured in fecal pellets, but less heterogeneity in RG representation in the luminal extracts of the cecum of these same individual colonized mice (FIG. 2D).

FIGS. 3A-3C demonstrate broad-spectrum oral antibiotic-conditioned mice raised under specific pathogen-free (SPF) conditions are colonized with diverse RG strains isolated from healthy and Lupus-affected human donors. The colonizing RG strains are then passed to their litters. The fecal pellets of SPF-raised C57BL/6 breeding pairs were collected before initiating an oral regimen of broad-spectrum antibiotics and repeated four weeks later, with total 16S rDNA genomic levels measured by qPCR analysis of extracts of fecal pellets (FIG. 3A). Fecal pellets from these same mice were evaluated for levels of RG-specific genomic DNA, before and after gavage with freshly cultured RG1, RG2 and S107-48 RG strains (FIG. 3B). After mating of pairs based on same RG strain, abundance of RG genomic DNA in fecal pellets of 5-week-old litters was measured in each of the SPF raised antibiotic preconditioned littermates then monocolonized by RG1, RG2, S107-48 or S47-18 strains, by RG-species specific 16S rDNA genomic DNA by qPCR analysis (FIG. 3C).

FIGS. 4A-4B show intestinal colonization of GF and SPF mice by select RG strains induces enhanced intestinal permeability. Neonatal intestinal colonization of the litters of GF mice by RG strains, RG2 and S107-48 but not the RG1 strain, induced increased intestinal permeability when tested by the fluorescein isothiocyanate (FITC)-dextran oral gavage challenge assay in mice at 10-14 weeks of age (FIG. 4A). Higher levels of intestinal permeability were found in RG2 and S107-48 strain colonized females than male littermates (FIG. 4B). Litters of SPF mice that were colonized by the RG strains, RG2 and S107-48, displayed increased intestinal permeability. Due to heterogeneity in responses, the group colonized by the S47-48 RG strain did not attain significance (FIG. 4C). Female mice colonized with RG2 and S107-48 displayed significantly greater levels of intestinal permeability than male mice or non-colonized control mice (FIG. 4D). Results indicate mean±standard error of the mean (SEM) (n=3-6 mice per group).

FIG. 5 depicts translocation of S107-48 RG strain 16S rDNA gene DNA in female mice colonized by the S107-48 RG Lupus strain. Extracts of draining mesenteric lymph nodes (MLN) from female 14-week-old littermates from GF mice colonized with the S107-48 RG strain demonstrated translocated RG genomic DNA (p=0.001). Levels in females were significantly higher than in males (p=0.002).

FIGS. 6A-6H show intestinal colonization with certain RG strains induces IgG RG strain-associated cell wall lipoglycan antibodies and anti-native DNA autoantibodies. Following neonatal colonization of litters from RG colonized GF breeding pairs, S107-48 RG strain colonized mice display numerically higher mean serum total IgG levels, compared to controls (FIG. 6A). Neonatal colonization with the RG2 strain (FIG. 6B) and the S107-48 RG strain (FIG. 6C) induced significantly raised serum levels of IgG anti-RG2 strain cell wall lipoglycan antibodies. Neonatal RG colonization did not induce raised IgG-antibody levels to: Salmonella LPS (FIG. 6D), Klebsiella LPS (FIG. 6E), or to pneumococcal cell wall polysaccharide (CWPS) (FIG. 6F). Neonatal colonization with the S107-48 RG strain induces raised serum levels of IgG anti-native DNA autoantibodies (FIG. 6G). Elevation of IgG anti native DNA following neonatal S107-48 RG strain was greatest in the female mice (FIG. 6H). (n=4 to 11 per group). Antibody assays for IgG anti-native DNA used plasma diluted at 1:100 in ELISA (OD₄₅₀). Antibody assays for IgG antibodies to RG lipoglycans, LPS and cell wall polysaccharide (CWPS) were performed by bead-based multiplex array, with relative antibody binding reactivity shown by mean fluorescence intensity (MFI) in murine plasma from these mice diluted 1:1000.

FIG. 7 shows correlation of levels of IgG anti-RG2 strain cell wall lipoglycan antibodies and IgG anti-native DNA autoantibodies correlate with levels of RG induced increased intestinal permeability. Each point represents an individual male and female littermate that had been neonatally colonized with S107-48 RG strain from a GF breeding pair, as depicted in FIG. 5, and FIG. 6B and FIG. 6G, respectively. Results from Spearman correlation analysis. IgG anti-RG2 plasma tested at 1:1000 dilution, and IgG anti-dsDNA was tested at a 1:100 dilution.

FIG. 8 shows neonatal RG colonization causes mild morphologic intestinal abnormalities with villous flattening. Representative hematoxylin and eosin (H&E) staining of terminal ileum sections from litters of RG-colonized and control or GF mice, with comparisons of mice sacrificed at 14 weeks of age (n=4 mice/group).

FIGS. 9A-9E show oral larazotide treatment normalizes abnormalities associated with gut permeability induced by RG strain neonatal colonization. Plasma zonulin levels in 14-week-old mice after neonatal colonization were significantly elevated in S107-48 RG strain mice compared to non-colonized controls (n=5 to 12 mice). For each group mean+/−standard deviation (SD) (FIG. 9A). In littermates from RG colonized SPF breeding pairs, individual mice were retested with results shown (before), then after a ten-day treatment with larazotide peptide in the water supply and 48-hour rest, gut permeability was then retested (after), with plasma FD4 (fluorescein isothiocyanate-dextran) levels shown for: RG2 strain colonized mice (FIG. 9B), S107-48 RG Lupus strain colonized mice (FIG. 9C), S47-18 RG Lupus strain colonized mice (FIG. 9D), with comparison to levels detected in non-colonized control mice (FIG. 9E). For the bottom panels, significance was tested by paired t test. *p<0.05 was considered statistically significant.

FIG. 10 shows an exemplary experimental timeline under germ-free (GF) (top panel) and under specific pathogen-free (bottom panel) conditions.

FIGS. 11A-11D depict dysbiosis and longitudinal instability in Systemic Lupus Erythematosus (SLE) microbiota communities compared to healthy individuals. Principal Coordinates Analysis (PCoA) was used to estimate beta diversity between groups using the Jensen-Shannon divergence (JSD) dissimilarity metric. Commensal communities from SLE patients were heterogenous, with most exhibiting significant distance variance from control (CTL) (multivariate distance Welch W*d test, p=0.001) (FIG. 11A). Compared to CTL, variance in diversity was greatest within the SLE disease activity index (SLEDAI) high subgroup (FIG. 11B), and the subset with active renal disease (FIG. 11C). To compare the overall dynamics of shifts in fecal communities sampled over time in different subjects, subject variances were computed based on JSD using the average multivariate dissimilarity estimation method reported in (Hamidi et al., 2019). The variances in these three groups were significantly different (Kruskal-Wallis ANOVA, p=0.03). Patients with a history of nephritis were assigned to the renal group based on American College of Rheumatology (ACR) criteria, whereas the patients in the non-renal group were without a history of documented Lupus nephritis (LN) based on ACR criteria. Lupus patients had more unstable gut microbiota than healthy individuals. Variance of gut microbiota was significantly different in the renal Lupus group, compared to healthy subjects (two-sided Mann-Whitney test, p=0.02). The non-renal Lupus group was also significantly different than the healthy subjects (p=0.03). The overall variance in the renal group was not different from the non-renal group (p=0.379, NS [not significant]) (FIG. 11D).

FIGS. 12A-12C depict dynamic changes in RG abundance documented at sequential time points in healthy and Lupus-affected individuals. All healthy control subjects displayed a stable low abundance in RG representation (FIG. 12A). In 11 of the 16 SLE patients under investigation, a stable low abundance in RG representation was detected (FIG. 12B). In 5/16 (31%) of the SLE patients evaluated over time, the abundance of RG fluctuated greatly over time (FIG. 12C). In these cases, RG abundance was at substantially higher levels in fecal samples obtained proximal to visits in which disease flares were documented. All but one of these RG bloom-associated patients had documented LN. RG relative abundance was evaluated for 16 SLE in 44 samples obtained at different time points, and for CTL subjects in 49 samples obtained at different time points, which ranged from 2-12 samples per donor. Clinical and demographic data are shown in Tables 1-6. Dotted line depicts an arbitrary 1% threshold of 16S rDNA amplicon representing RG abundance that is highly above the mean 0.15% level of abundance in these healthy controls. Abundance levels above 1% were considered a bloom. Note that in panel C, for patient S78 the greater range of RG abundance necessitated a different scale. SLE disease activity index (SLEDAI)>8 was considered high disease activity.

FIGS. 13A-13H depict analyses of Ruminococcus blautia gnavus (RG) isolate whole genomes from two Lupus patients in clinical flare. Long-read assemblies of five RG isolates. From inside to outside, circular tracks show GC content within 1 kbp (kilobase pair) windows (black line), assembled contigs (gray rectangles), locations of 1 kbp windows with BLAST hits to type strains RG1 (ATCC29149) and RG2 (CC55-00 1C) genome assemblies downloaded from RefSeq, and locations of genes as predicted and annotated by Prokka. Raw sequence data and assemblies deposited to NCBI repositories under BioProject PRJNA821229 (FIGS. 13A-13E). Dendrogram showing hierarchical clustering of the gene content (orthogroup presence/absence) of newly sequenced RG isolates together with assemblies downloaded from NCBI RefSeq (FIG. 13F-13H).

FIGS. 14A-14I show lipoglycan in different RG strains obtained from Lupus donors during nephritis flares are antigenically and structurally related. Molecular components of different RG strains were separated by polyacrylamide gel electrophoresis, transferred to membranes, and immunoblotted with serum IgG from patient S47, obtained during a disease flare. Strains isolated from Lupus patient S107 (S107-48, S107-61 and S107-86), and from patient S47 (S47-18), while RJX1118 was from an antibiotic-treated infant and RJX1125 strain was from a patient with irritable bowel disease (IBD) hall (Hall et al., 2017), and RG1 and MSK22.24 strains were from healthy donors (FIG. 14A). Aliquots of these same bacterial extracts were treated with proteinase K, which revealed persistence of the immune recognition by Lupus serum IgG of the oligobands of these non-protein antigens (FIG. 14B). Nine different RG strains from IBD patients, and two RG strains from antibiotic-treated infants, did not contain the immunoreactive oligobanding pattern detected by immunoblotting of the lipoglycan found in RG2 extract and purified RG2 lipoglycan. These strains and their genomes are described in (Hall et al., 2017). Non-reactive RG1 (without the pattern of immunoreactivity that is a characteristic of the lipoglycan) was also originally isolated from a healthy adult (FIG. 14C). Immunoblot reactivity of S47 patient's serum IgG-antibodies were reactive with extracts of five RG strains, and the lipoglycan purified from Lupus S47-18 strain (FIG. 14D). A replicate immunoblot was performed after preincubation of the S47 sera with 4 μg of purified S47-18 LG, which resulted in complete inhibition of Lupus serum IgG reactivity with oligobands of comparable molecular weight (MW) in S107-48, S107-86 and S47-18 extracts and the purified S47-18 LG. The lower MW band in RG1 from a healthy donor, which was previously shown to be protease sensitive, was unaffected. Lipoglycan oligobands migrate to an area delineated by the boxes (FIG. 14E). Heatmap of the 100 most abundant signals in the range of 2500-5000 Da of LGs from strains RG2, S107-86, and S47-18, respectively (corresponding mass spectra are shown in FIG. 26) (FIGS. 14F-14G). Isotope clusters originating from the same molecular species are grouped by bracket and the LG structural composition is assigned if applicable. Spectral similarity score calculated from these 100 most abundant peaks indicated a high similarity between the LG populations of these three strains (p<2×10⁻¹²) (FIG. 14H). Structure model of the major abundant LG species consisting of tri-acylated LG, di-acylated LG and mono-acyl LG. The glycoconjugate consists of a diacylglycerol-hexuronic acid linker and the next two connected sugars are hexoses. The lipid anchor can potentially include one or both hexoses. The third fatty acid can be either bond to the hexuronic acid, the two adjacent hexoses or to sugar moiety of the core glycan. While the core glycan composition of the purified LG of the three analyzed RG strains showed a remarkable conserved composition, this core glycan can be extended by further hexoses. This depiction was created with Biorender.com software (FIG. 14I).

FIGS. 15A-15C demonstrate reactivity of post-immunization murine monoclonal antibodies is restricted to conserved cross-reactive determinants associated with the oligobands of protease-resistant lipoglycans from RG strains isolated from clinically active lupus nephritis (LN) patients. Direct binding enzyme-linked immunosorbent assay (ELISA) demonstrates reactivity of both mAbs, 33.2.3 and 34.2.2, with the purified RG2 and S47-18 LGs, and bacterial extracts from the RG2 strain and Lupus-derived strains; S47-18, S107-48 and S107-86. In this assay, LG or nuclease-treated RG strain extracts were precoated overnight onto the well, then after blocking with bovine serum albumin, the mAbs, or isotype control at 1 μg/ml were incubated for 4 hours, then washed and developed (see below-described methods) (FIG. 15A). Immunoblot with mAb 33.2.3 (FIG. 15B) and immunoblot with 34.2.2 mAb (FIG. 15C), detect antigenically related LG oligobands in extracts of R. gnavus strains isolated from active Lupus nephritis strains. In each panel is shown samples of purified lipoglycan from the Lupus S47-18 strain, and the RG2 strain of R. gnavus. At left, extracts of whole bacteria are shown for the RG1 strain (from a healthy donor), and the Lupus-derived strains; S107-48, S107-86, S47-18, and for the RG2 strain.

FIGS. 16A-16L demonstrate serum IgG antibodies to Lupus RG strain LGs, parallel gut community abundance, peak with RG blooms and concordant disease flares. To investigate the Lupus host immune response overtime to colonization with RG strains, IgG anti-RG antibody responses were studied in longitudinally obtained sera from three Lupus patients, with titration of antibody binding reactivity. Results for IgG-antibody responses of patient S47 over time with different RG strains or purified RG strain LGs (FIGS. 16A-16D). For patient 61 (FIGS. 16E-16H). For patient S78 (FIGS. 16I-16L). Serum IgG-reactivity at multiple dilutions was assessed for binding to whole bacterial extract of RG2, the index RG strains that contained the first identified immunogenic LG (FIG. 16A, FIG. 16E and FIG. 16I). Comparisons with purified LG from this same RG2 strain (i.e., RG2 LG) (FIG. 16B, FIG. 16F, and FIG. 16J), which show very similar reactivity patterns, confirming the high immunogenicity of LG within the whole bacterial extract. Reactivity with the structurally related LG (see, e.g., FIG. 14C) from the S47-18 strain (S47-18 LG) that came from the S47 LN donor (FIG. 16C, FIG. 16G and FIG. 16K). The same relative reactivity patterns are seen, although uniformly stronger levels were seen with sera from each of the three patients with this RG strain LG (i.e., IgG-antibody binding curves for S47-18 LG are shifted up and to the right). By contrast, there is little or no reactivity with the LPS glycan from a Pseudomonas species, which is not known to be a blooming bacterial species within the microbiota communities in these subjects (FIG. 16D, FIG. 16H, and FIG. 16L). In each panel, reactivity with serum from a representative healthy female control is shown (CTL). Samples obtained sequentially over time are indicated accordingly. Studies performed with custom bead based MBI array (Luminex), as described in Example 7.

FIG. 17 demonstrates consistent library composition between the two 16S rDNA gene amplimer Miseq batches. Principal Coordinates Analysis (PCoA) on libraries generated from samples of same individuals/samples sequenced in the two different 16S rDNA sequencing runs (1^{st} and 2^{nd}) as an inter-run technical control, shows no consistent bias or batch effect.

FIGS. 18A-18C show dysbiosis in SLE microbiota communities. The number of distinct taxa were estimated based on observed Amplicon Sequence Variants (ASVs), and alpha diversity richness was reduced in samples from Lupus patients (SLE) compared to healthy controls (CTL) (Wilcoxon, p=0.0023) (FIG. 18A). Compared to CTL, alpha diversity was reduced in Lupus patients with low disease activity (based on SLEDAI score), with even greater contractions in the group with high disease activity, (Wilcoxon, p=0.034, p=0.0045, respectively) (FIG. 18B). Compared to CTL, alpha diversity was reduced in patients with inactive renal disease, and further reduced in patients with active renal disease (Wilcoxon, p=0.0006 and p=0.0033, respectively) (FIG. 18C). The purpose was to assess whether there were correlations that in an individual subject might be constant or that vary over time. These analyses did not consider statistical adjustments for multiple samples from the same patient.

FIGS. 19A-19C demonstrate alpha diversity is reduced in libraries from patients with high Lupus disease activity. Analyses performed as shown in FIG. 11. High disease activity was defined as a composite SLEDAI score of >8.

FIGS. 20A-20B demonstrate alpha diversity is reduced in libraries from patients with active renal disease compared to inactive renal disease. Analyses performed as shown in FIG. 12. Active renal disease was defined by standard clinical laboratory criteria.

FIGS. 21A-21C show RG expansions occur at the time of high Lupus disease activity and active LN. All samples from SLE patients had a numerical trend toward increased RG abundance compared to healthy CTL (Wilcoxon, p=0.0760) (FIG. 21A). Samples from patients with high disease activity (based on SLEDAI) showed a greater RG abundance, compared to low disease activity and CTL (Wilcoxon, p=0.81, p=0.01, respectively) (FIG. 21B). RG expansions were common in the active LN group compared to healthy CTL (Wilcoxon, p=0.02), but not significantly different in the inactive LN group (Wilcoxon, p=0.27, NS) (FIG. 21C). RG relative abundance is shown in log 2 values.

FIGS. 22A-22C show blooms of bacteria of the Veillonella family do not occur concurrent with episodes of higher Lupus disease activity. Veillonella abundance in healthy individuals (FIG. 22A). Veillonella abundance in SLE patients (FIG. 22B). Veillonella abundance in SLE patients with above-described RG blooms concordant with Lupus disease activity flares (FIG. 22C). Abundance based on ASV representation in total amplicon libraries.

FIGS. 23A-23C show blooms of Fusobacterium that do not occur concurrent with peak flares of Lupus disease activity. Fusobacterium abundance in healthy individuals (FIG. 23A). Fusobacterium abundance in SLE patients (FIG. 23B). Fusobacterium abundance in SLE patients with above-described RG blooms concordant with Lupus disease activity flares (FIG. 23C). Abundance based on ASV representation in total amplicon libraries.

FIG. 24 displays a heat map at the species level showing dominance of Blautia (Ruminococcus) gnavus (RG) in many patients with active LN. The abundance of the species in the different groups (healthy controls, CTL), patients with active nephritis and with inactive nephritis, by using the Z-score. Cut-offs were used that were calculated based on the Z-score. Taxa with a mean relative abundance of greater than 0.001 are depicted, and taxa with DEseq2 padj>0.1 across all samples. The higher-level abundance of R. gnavus and paucity of Faecalibacterium bacteria is evident in communities of Lupus patients with active disease.

FIGS. 25A-25C show a phylogenetic tree based on core alignment of Blautia (Ruminococcus) gnavus (RG) genome assemblies downloaded from NCBI RefSeq, together with five newly generated genome assemblies reported here. Newly sequenced isolates are shown in bold, along with the RG1 (ATCC 29149) strain genome for reference.

FIG. 26 shows charge-deconvoluted spectra of the mass spectrometric (MS) analysis of LGs from RG-strains RG2 (top), S107-86 (middle) and S47-28 (bottom) performed in the negative ion mode. Details of all detected molecular LG species for strain RG2 are summarized in Table 7. Relative abundance for the depicted spectral region was normalized to the respective base peak (tri-acyl LG 47:0).

FIG. 27 depicts MS² analysis of the basic de-O-acyl LG without hexose extension (composed of 1 Gro, 8 Hex, 5 HexNAc, 3 HexU; calc. mono-isotopic mass: 2931.963 Da) of strain RG2 obtained after hydrazine-treatment. In the experiment shown here, the double charged peak for this molecule was isolated and an NCE of 30 applied to induce fragmentation. Full spectrum is depicted in the upper panel. A zoom into the region of m z 140 to 650 comprising the small molecular fragments generated under these conditions is depicted below. Fragments indicative of the presence of a glycerol-hexuronic acid unit (221.0665 Da: glycerol-hexuronic acid [decarboxylated]; 249.0614 Da: glycerol-hexuronic acid, with loss of water) which was extended by at least two hexoses (545.1723 Da: glycerol-hexuronic acid [decarboxylated]-hexose-hexose) can be observed. The loss of the glycerol-hexuronic acid unit including loss of water can also be observed from the selected ion. (z=1, single charged ions [M−H]⁺; z=2, double charged ions [M−2H]²⁻).

FIGS. 28A-28B show variable region sequences of the 33.2.2 murine monoclonal antibody. Both nucleic acid and deduced protein sequences for VH region (FIG. 28A) and VL region (FIG. 28B) are shown. FIG. 28A discloses SEQ ID NOs 4-5, respectively, in order of appearance. FIG. 28B discloses SEQ ID NOs 9-10, respectively, in order of appearance. The CDR sequences according to the IMGT numbering system are shown.

FIG. 29 shows polyacrylamide gene analysis of purified chimeric antibody product, under non-reducing (left) and reducing conditions (right). The percentage of polyacrylamide in each gel is indicated at the bottom.

FIG. 30 shows chromatogram overlay of tracings from size exclusion chromatography (SEC)-high performance chromatography (HPLC). These results document that a single peak was obtained after purification, which had a molecular weight estimated at ˜158 kd (size marker not included) which was without detectable aggregates.

FIG. 31 shows binding activity of the 33.2.2 chimeric IgG2 antibody to R. gnavus lipoglycan immobilized onto commercial paramagnetic beads. Each point is the mean of duplicate measurements for this condition. Binding activity was detected with a signal above background for the assay with an IgG concentration of below 400 μg/ml, with activity also documented that was below saturation of the assay even when using 1500 ng/ml of the chimeric antibody.

FIGS. 32A-32B show variable region sequences of the 34.2.2 murine monoclonal antibody. Both nucleic acid and deduced protein sequences for VH region (FIG. 32A) and VL region (FIG. 32B) are shown. FIG. 32A discloses SEQ ID NOs 13-14, respectively, in order of appearance. FIG. 32B discloses SEQ ID NOs 15-16, respectively, in order of appearance. The CDR sequences according to the IMGT numbering system are shown.

### DETAILED DESCRIPTION

### Definitions

The term “antibody” refers to an immunoglobulin molecule capable of specific binding to a target, such as a carbohydrate, polynucleotide, lipid, polypeptide, etc., through at least one antigen recognition site, located in the variable region(s) of the immunoglobulin molecule. As used herein, the term “antibody”, e.g., anti-lipoglycan antibody, encompasses not only intact (e.g., full-length) polyclonal or monoclonal antibodies, but also antigen-binding fragments thereof (such as Fab, Fab, F(ab′)2, Fv), single chain (scFv), mutants thereof, fusion proteins comprising an antibody portion, humanized antibodies, chimeric antibodies, diabodies, nanobodies, linear antibodies, single chain antibodies, multi-specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-lipoglycan antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (VH) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, the EU definition, the “Contact” numbering scheme, the “IMGT” numbering scheme, the “AHo” numbering scheme, and/or the contact definition, all of which are well known in the art. (See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; Edelman et al., Proc Natl Acad Sci USA. 1969 May; 63(1):78-85; and Almagro, J. Mol. Recognit. 17:132-143 (2004); MacCallum et al., J. Mol. Biol. 262:732-745 (1996), Lefranc M P et al., Dev Comp Immunol, 2003 January; 27(1):55-77; and Honegger A and Pluckthun A, J Mol Biol, 2001 Jun. 8; 309(3):657-70. See also hgmp.mrc.ac.uk and bioinf.org.uk/abs).

In some embodiments, the anti-lipoglycan antibody described herein is a full-length antibody, which contains two heavy chains and two light chains, each including a variable domain and a constant domain. Alternatively, the anti-lipoglycan antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, CL and CH1 domains; (ii) a F(ab′)₂ fragment, a bivalent fragment including two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

Any of the antibodies described herein, e.g., anti-lipoglycan antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

As used herein “specifically binds”, “specific binding”, “specifically recognizes” or “specifically recognition” refers to the ability of the antibodies or antigen-binding fragments of the disclosure to bind to a predetermined antigen (e.g., a lipoglycan-containing antigen associated with Ruminococcus gnavus strain CC55_001C/HM-1056, S107-86, S47-18, and/or S107-48) with a dissociation constant (K_{D}) of about 1×10⁻⁶ M or less, for example about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. Typically, the antibody or antigen-binding fragment binds to an antigen (e.g., a lipoglycan-containing antigen associated with Ruminococcus gnavus strain CC55_001C/HM-1056, S107-86, S47-18, and/or S107-48) with a K_{D} that is at least ten-fold less than its K_{D} for a nonspecific antigen (for example BSA or casein) as measured by surface plasmon resonance using for example a Proteon Instrument (BioRad).

“Isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids in the polynucleotides that encode for the antibody or antigen-binding fragment described herein. An “isolated” antibody or antigen-binding fragment, as used herein, is intended to refer to an antibody or antigen-binding fragment which is substantially free of other antibodies or antigen-binding fragments having different antigenic specificities (for instance, an isolated antibody that specifically binds to an intended antigen (e.g., a lipoglycan-containing antigen associated with Ruminococcus gnavus strain CC55_001C/HM-1056, S107-86, S47-18, and/or S107-48) is substantially free of antibodies that specifically bind antigens other than the intended antigen (e.g., a lipoglycan-containing antigen associated with Ruminococcus gnavus strain CC55_001C/HM-1056, S107-86, S47-18, and/or S107-48).

As used herein, the terms “microbe” or “microorganism” encompass both prokaryotic organisms including bacteria and archaea, and eukaryotic organisms, including fungi, present as components of the mammalian microbiota, and viruses.

The terms “gastrointestinal microbiota”, “GI microbiota”, “intestinal microbiota”, “intestinal flora”, and “intestinal microbiome” are used interchangeably and refer to the microorganisms that colonize the intestines in an individual.

As used herein, the term “dysbiosis” refers to a microbial imbalance on or inside the body. Dysbiosis can result from, e.g., antibiotic exposure as well as other causes, e.g., infections with pathogens including viruses, bacteria and eukaryotic parasites.

Specific taxa and changes in GI microbiota discussed herein can be detected using various methods, including without limitation quantitative PCR or high-throughput sequencing methods which detect over- and under-represented genes in the total bacterial population (e.g., sequencing for microbial community analysis (e.g., using a 454 machine or Illumina Miseq or other related devices); screening of microbial 16S ribosomal RNA genes (16S rRNA) or also called the microbial 16S ribosomal DNA (16S rDNA), next generation sequencing (NGS) etc.), or transcriptomic or proteomic studies that identify absent (or under-represented) or gained or over-represented microbial transcripts or proteins within total bacterial populations. See, e.g., U.S. Patent Publication No. 2010/0074872; Eckburg et al., Science, 2005, 308:1635-8; Costello et al., Science, 2009, 326:1694-7; Grice et al., Science, 2009, 324:1190-2; Li et al., Nature, 2010, 464: 59-65; Bjursell et al., Journal of Biological Chemistry, 2006, 281:36269-36279; Mahowald et al., PNAS, 2009, 14:5859-5864; Wikoff et al., PNAS, 2009, 10:3698-3703.

As used herein, the term “16S rDNA sequencing” refers to the sequencing of 16S ribosomal DNA (rDNA) or 16S ribosomal RNA (rRNA) gene sequences by using primers such as universal primers (i.e., for amplifying all sequence variants present in different bacterial species) and/or species-specific primers to identify the bacteria species of interest present in a sample. rDNA and rRNA genes contain both highly conserved sites and hypervariable regions, and the latter can provide species-specific signature sequences useful for identification and abundance of individual bacteria species. Such universal primers are well known in the art.

As used herein, the term “operational taxonomic unit” or “OTU” refers to group of bacterial sequences that differ among each other as each shares<97% identity. The term “operational taxonomic unit” or “OTU” can be used to identify different bacterial species and their relative abundance in a sample. A “type” or a plurality of “types” of bacteria includes an OTU or a plurality of different OTUs, and also encompasses differences assignable to species, genus, family or order of bacteria. The specific genetic sequence may be the 16S rDNA or rRNA sequence or a portion of the 16S rDNA or rRNA sequence or it may be a functionally conserved housekeeping gene found broadly across the eubacterial kingdom.

As used herein, the term “abundance” refers to how common or rare a particular organism (e.g., bacterial species) is relative to other organisms of the same type (e.g., other bacterial species) in a defined community. In certain embodiments, abundance is the percent composition of a particular organism (e.g., bacterial species) to the total amount of organisms in the sample. In certain embodiments, abundance refers to the total level of organism in a sample. In certain embodiments, abundances refers to the percent composition of a particular organism (e.g., bacterial species) to the total amount of organisms from the same trophic level.

As used herein, the term “probiotic” refers to a substantially pure bacteria (i.e., a single isolate, or homogeneous culture of, e.g., live bacterial cells, conditionally lethal bacterial cells, inactivated bacterial cells, killed bacterial cells, spores, recombinant carrier strains), or a mixture of desired bacteria, bacteria components or bacterial extract, or bacterially-derived products (natural or synthetic bacterially-derived products such as, e.g., bacterial antigens or metabolic products) and may also include any additional components that can be administered to a mammal. Such compositions are also referred to herein as “bacterial inoculants” or “microbiota inoculants”. Probiotics or bacterial inoculant compositions of the invention may be administered after dispersion in a buffering agent to allow the bacteria to survive in the acidic environment of the stomach, i.e., to resist low pH and to grow in the intestinal environment. Such buffering agents include sodium bicarbonate, juice, milk, yogurt, infant formula, and other dairy products.

As used herein, the term “prebiotic” refers to an agent that increases the number, the abundance and/or activity of one or more desired bacteria, enhancing their growth. Non-limiting examples of prebiotics useful in the methods of the present invention include fructooligosaccharides (e.g., oligofructose, inulin, inulin-type fructans), galactooligosaccharides, human milk oligosaccharides (HMO), Lacto-N-neotetraose, D-Tagatose, xylo-oligosaccharides (XOS), arabinoxylan-oligosaccharides (AXOS), N-acetylglucosamine, N-acetylgalactosamine, glucose, other five- and six-carbon sugars (such as arabinose, maltose, lactose, sucrose, cellobiose, etc.), amino acids, alcohols, resistant starch (RS), water-soluble cellulose derivatives (most preferably, methylcellulose, methyl ethyl cellulose, hydroxyethyl cellulose, ethyl hydroxyethyl cellulose, cationic hydroxyethyl cellulose, hydroxypropyl cellulose, hydroxyethyl methylcellulose, hydroxypropyl methylcellulose, and carboxymethyl cellulose), water-insoluble cellulose derivatives (most preferably, ethyl cellulose), and mixtures thereof. See, e.g., Ramirez-Farias et al., Br J Nutr (2008) 4:1-10; Pool-Zobel and Sauer, J Nutr (2007), 137:2580S-2584S.

The term “polynucleotide” as referred to herein means a polymeric form of nucleotides of at least 10 bases in length, either ribonucleotides or deoxyribonucleotides or a modified form of either type of nucleotide. The term includes single and double stranded forms.

The term “isolated polynucleotide” as used herein means a polynucleotide of genomic, cDNA, or synthetic origin or some combination thereof, which by virtue of its origin or source of derivation, the “isolated polynucleotide” has one to three of the following: (1) is not associated with all or a portion of a polynucleotides with which the “isolated polynucleotide” is found in nature, (2) is operably linked to a polynucleotide to which it is not linked in nature, or (3) does not occur in nature as part of a larger sequence.

“Operably linked” sequences include both expression control sequences that are contiguous with the gene of interest and expression control sequences that act in trans or at a distance to control the gene of interest. The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “vector”, as used herein, means a vehicle capable of transporting a nucleic acid into a host cell. In some embodiments, the vector is a plasmid, i.e., a circular double stranded DNA loop into which additional DNA segments may be ligated. In some embodiments, the vector is a viral vector, wherein additional DNA segments may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

The term “promoter” as used herein is defined as a DNA sequence recognized by the synthetic machinery of the cell, or introduced synthetic machinery, required to initiate the specific transcription of a polynucleotide sequence. As used herein, the term “regulatory sequence” means a nucleic acid sequence which can regulate expression of a gene product operably linked to the regulatory sequence. In some instances, this sequence may be the core promoter sequence and in other instances, this sequence may also include an enhancer sequence and other regulatory elements which are required for expression of the gene product. The promoter or regulatory sequence may, for example, be one which expresses the gene product in a tissue specific manner.

The term “recombinant host cell” (or simply “host cell”), as used herein, means a cell into which an exogenous nucleic acid and/or recombinant vector has been introduced. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

The term “percent sequence identity” means a ratio, expressed as a percent of the number of identical residues over the total number of residues compared.

Sequence identity for nucleic acid sequences may be analyzed over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183:63-98 (1990); Pearson, Methods Mol. Biol. 132:185-219 (2000); Pearson, Methods Enzymol. 266:227-258 (1996); Pearson, J. Mol. Biol. 276:71-84 (1998); herein incorporated by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference.

A reference to a nucleotide sequence encompasses its complement unless otherwise specified. Thus, a reference to a nucleic acid having a particular sequence should be understood to encompass its complementary strand, with its complementary sequence.

Sequence identity for polypeptides, is typically measured using sequence analysis software. Protein analysis software matches sequences using measures of similarity assigned to various substitutions, deletions and other modifications, including conservative amino acid substitutions. For instance, GCG contains programs such as “Gap” and “Bestfit” which can be used with default parameters, as specified with the programs, to determine sequence homology or sequence identity between closely related polypeptides, such as homologous polypeptides from different species of organisms or between a wild-type protein and a mutein thereof. See, e.g., GCG Version 6.1. Polypeptide sequences also can be compared using FASTA using default or recommended parameters, see GCG Version 6.1. (University of Wisconsin Wis.) FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). Another preferred algorithm when comparing a sequence of the invention to a database containing a large number of sequences from different organisms is the computer program BLAST, especially blastp or tblastn, using default parameters, as supplied with the programs. See, e.g., Altschul et al., J. Mol. Biol. 215:403-410 (1990); Altschul et al., Nucleic Acids Res. 25:3389-402 (1997).

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences.

The term “substantial similarity” or “substantial sequence similarity,” when referring to a nucleic acid or fragment thereof, means that when optimally aligned with appropriate nucleotide insertions or deletions with another nucleic acid (or its complementary strand), there is nucleotide sequence identity in at least about 85%, preferably at least about 90%, 91%, 92%, 93%, 94%, 95%, 96%, 97%, 98% or 99% of the nucleotide bases, as measured by any well-known algorithm of sequence identity, such as FASTA, BLAST or Gap, as discussed above.

As applied to polypeptides, the term “substantial identity” means that two peptide sequences, when optimally aligned, such as by the programs GAP or BESTFIT using default gap weights, as supplied with the programs, share at least 70%, 75%, 80% or 85% sequence identity, preferably at least 90%, 91%, 92%, 93%, 94% 95%, 96%, 97%, 98% or 99% sequence identity. In certain embodiments, residue positions that are not identical differ by conservative amino acid substitutions.

A “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity may be adjusted upwards to correct for the conservative nature of the substitution. Means for making this adjustment are well-known to those of skill in the art. See, e.g., Pearson, Methods Mol. Biol. 243:307-31 (1994). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine.

Alternatively, a conservative substitution or replacement, as the terms are used interchangeably herein, is any change having a positive value in the PAM250 log-likelihood matrix disclosed in Gonnet et al., Science 256:1443-45 (1992), herein incorporated by reference. A “moderately conservative” replacement is any change having a nonnegative value in the PAM250 log-likelihood matrix.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein, the term “therapeutically effective amount” refers to the amount of a compound (e.g., an anti-lipoglycan antibody), or a composition (including e.g., an anti-lipoglycan antibody, optionally with a prebiotic or a probiotic), that, when administered to a subject for treating (e.g., preventing or ameliorating) a state, disorder or condition, is sufficient to effect such treatment. The “therapeutically effective amount” will vary depending, e.g., on the compound, composition, bacteria or analogues administered as well as the disease, its severity, and physical conditions and responsiveness of the subject to be treated.

As used herein, the phrase “pharmaceutically acceptable” refers to molecular entities and compositions that are generally regarded as physiologically tolerable.

As used herein, the term “combination” of a compound, composition, bacterial inoculant, probiotic, analogue, or prebiotic and at least a second pharmaceutically active ingredient means at least two, but any desired combination of compound, composition, bacterial inoculant, probiotic, analogue, or prebiotic can be delivered simultaneously or sequentially (e.g., within a 24-hour period).

Within the meaning of the present invention, the term “conjoint biotic administration” is used to refer to administration of a probiotic and a prebiotic simultaneously in one composition, or simultaneously in different compositions, or sequentially (preferably, within a 24-hour period).

The terms “patient”, “individual”, “subject”, and “animal” are used interchangeably herein and refer to mammals, including, without limitation, human and veterinary animals (e.g., cats, dogs, cows, horses, goats, sheep, pigs, etc.) and experimental animal models. In a preferred embodiment, the subject is a human.

As used herein, the term “healthy subject” refers to a subject that is without known infections or autoimmune disorders by using conventional diagnostic methods. In certain embodiments, a healthy subject is a subject without a known first degree relative with an autoimmune disorder. In certain embodiments, a matched healthy subject is matched by age, gender, and/or ethnicity.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E.W. Martin.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terms “a,” “an,” and “the” do not denote a limitation of quantity, but rather denote the presence of “at least one” of the referenced item.

The practice of the present invention employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

### Antibodies and Antigen-Binding Fragments

In some embodiments, the present disclosure provides isolated antibodies, or antigen-binding fragments thereof, that specifically recognize a bacterial antigen associated with a Ruminococcus gnavus strain.

An antigen is a molecule capable of inducing an immune response in a host. For example, an antigen can be any substance, e.g., protein, carbohydrate, lipid, lipoglycan, nucleic acid, or a mixture or combination thereof, to which an immune response is elicited.

In certain embodiments, the bacterial antigen is derived from Ruminococcus gnavus strain CC55_001C/HM-1056 (Human Microbiome Project (HMP) ID 1201; GenBank: AZJF00000000; RG2), S107-86, 547-18, and/or S107-48.

In some embodiments, the Ruminococcus gnavus strain is CC55_001C/HM-1056 (Human Microbiome Project (HMP) ID 1201; GenBank: AZJF00000000; RG2).

Taxonomically, R. gnavus originally belonged to the genus Ruminococcus in the family Ruminococcaceae. The Ruminococcus genus contained 18 species, but on the basis of 16S ribosomal gene sequencing, some of the species have been reassigned to the new genus Blautia within the family Lachnospiraceae, which, like Ruminococcaceae, is a part of the order Clostridiales. R. gnavus was reassigned due to its active fermentative ability, but R. gnavus has retained the Ruminococcus genus name (Skerman et al., Int. J. Syst. Bacteriol. 1980).

In certain embodiments, the bacterial antigen is derived from a bacterial strain from the Lachnospiraceae family, wherein the strain has 16S rDNA or rRNA with at least 95%, 96%, 97%, 98%, 99% or including 100% sequence identity to the 16S rDNA or rRNA of Ruminococcus gnavus strain CC55_001C, S107-86, S47-18, and/or S107-48, as described above, over its entire length or at least 95%, 96%, 97%, 98%, 99% or including 100% sequence identity to any single V region of the 16S rDNA or rRNA. In certain embodiments, the bacterial antigen is derived from a bacterial strain from the Lachnospiraceae family, wherein the strain has 16S rDNA or rRNA with at least 95%, sequence identity to the 16S rDNA or rRNA of Ruminococcus gnavus strain CC55_001C, S107-86, S47-18, and/or S107-48 over its entire length. In certain embodiments, the bacterial antigen is derived from a bacterial strain from the Lachnospiraceae family, wherein the strain has at least 97% sequence identity to any single V region of the 16S rDNA or rRNA of Ruminococcus gnavus strain CC55_001C, S107-86, S47-18, and/or S107-48. In certain embodiments, the V region of 16S rDNA or rRNA is the V4 region. In certain embodiments, the bacterial antigen is derived from a bacterial strain from the Lachnospiraceae family, wherein the strain has at least 99% sequence identity to any single V region of the 16S rDNA or rRNA of Ruminococcus gnavus strain CC55_001C, S107-86, S47-18, and/or S107-48. In certain embodiments, the V region of 16S rDNA or rRNA is the V4 region.

In certain embodiments, the bacterial antigen is derived from a bacterial strain from the Blautia genus, wherein the strain has 16S rDNA or rRNA with at least 95%, 96%, 97%, 98%, 99% or including 100% sequence identity to the 16S rDNA or rRNA of Ruminococcus gnavus strain CC55_001C, S107-86, S47-18, and/or S107-48, as described above, over its entire length or at least 95%, 96%, 97%, 98%, 99% or including 100% sequence identity to any single V region of the 16S rDNA or rRNA. In certain embodiments, the bacterial antigen is derived from a bacterial strain from the Blautia genus, wherein the strain has 16S rDNA or rRNA with at least 95%, sequence identity to the 16S rDNA or rRNA of Ruminococcus gnavus strain CC55_001C, S107-86, S47-18, and/or S107-48 over its entire length. In certain embodiments, the bacterial antigen is derived from a bacterial strain from the Blautia genus, wherein the strain has at least 97% sequence identity to any single V region of the 16S rDNA or rRNA of Ruminococcus gnavus strain CC55_001C, S107-86, S47-18, and/or S107-48. In certain embodiments, the V region of 16S rDNA or rRNA is the V4 region. In certain embodiments, the bacterial antigen is derived from a bacterial strain from the Blautia genus, wherein the strain has at least 99% sequence identity to any single V region of the 16S rDNA or rRNA of Ruminococcus gnavus strain CC55_001C, S107-86, 547-18, and/or S107-48. In certain embodiments, the V region of 16S rDNA or rRNA is the V4 region.

In certain embodiments, the bacterial antigen is derived from a bacterial strain from the Ruminococcus gnavus species, wherein the strain has 16S rDNA or rRNA with at least 95%, 96%, 97%, 98%, 99% or including 100% sequence identity to the 16S rDNA or rRNA of Ruminococcus gnavus strain CC55_001C, S107-86, S47-18, and/or S107-48, as described above, over its entire length or at least 95%, 96%, 97%, 98%, 99% or including 100% sequence identity to any single V region of the 16S rDNA or rRNA. In certain embodiments, bacterial antigen is derived from a bacterial strain from the Ruminococcus gnavus species, wherein the strain has 16S rDNA or rRNA with at least 95%, sequence identity to the 16S rDNA or rRNA of Ruminococcus gnavus strain CC55_001C, S107-86, S47-18, and/or S107-48 over its entire length. In certain embodiments, the bacterial antigen is derived from a bacterial strain from the Ruminococcus gnavus species, wherein the strain has at least 97% sequence identity to any single V region of the 16S rDNA or rRNA of Ruminococcus gnavus strain CC55_001C, S107-86, S47-18, and/or S107-48. In certain embodiments, the V region of 16S rDNA or rRNA is the V4 region. In certain embodiments, the bacterial antigen is derived from a bacterial strain from the Ruminococcus gnavus species, wherein the strain has at least 99% sequence identity to any single V region of the 16S rDNA or rRNA of Ruminococcus gnavus strain CC55_001C, S107-86, S47-18, and/or S107-48. In certain embodiments, the V region of 16S rDNA or rRNA is the V4 region.

In certain embodiments, the bacterial antigen comprises a non-protein, non-nucleic acid molecule. In certain embodiments, the bacterial antigen comprises a bacterial lipoglycan or a derivative thereof.

In addition to full-length lipoglycan, antibodies or antigen binding fragments of the disclosure may bind to lipoglycan derivatives, including lipoglycan fragments. Production of such lipoglycan fragments is disclosed, e.g., in van der Es et al., Chem Soc Rev. 2017 46(5):1464-1482, which is incorporated herein by reference in its entirety.

In certain embodiments, the lipoglycan-containing antigen or derivative thereof is derived from Ruminococcus gnavus strain CC55_001C/HM-1056 (Human Microbiome Project (HMP) ID 1201; GenBank: AZJF00000000), S107-86, S47-18, and/or S107-48.

In certain embodiments, the lipoglycan-containing antigen or derivative thereof is derived from a bacterial strain from the Lachnospiraceae family, wherein the strain has 16S rDNA or rRNA with at least 95%, 96%, 97%, 98%, 99% or including 100% sequence identity to the 16S rDNA or rRNA of Ruminococcus gnavus strain CC55_001C, S107-86, 547-18, and/or S107-48, as described above, over its entire length or at least 95%, 96%, 97%, 98%, 99% or including 100% sequence identity to any single V region of the 16S rDNA or rRNA. In certain embodiments, the lipoglycan-containing antigen or derivative thereof is derived from a bacterial strain from the Lachnospiraceae family, wherein the strain has 16S rDNA or rRNA with at least 95%, sequence identity to the 16S rDNA or rRNA of Ruminococcus gnavus strain CC55_001C, S107-86, S47-18, and/or S107-48 over its entire length. In certain embodiments, the lipoglycan or derivative thereof is derived from a bacterial strain from the Lachnospiraceae family, wherein the strain has at least 97% sequence identity to any single V region of the 16S rDNA or rRNA of Ruminococcus gnavus strain CC55_001C, S107-86, S47-18, and/or S107-48. In certain embodiments, the V region of 16S rDNA or rRNA is the V4 region. In certain embodiments, the lipoglycan-containing antigen or derivative thereof is derived from a bacterial strain from the Lachnospiraceae family, wherein the strain has at least 99% sequence identity to any single V region of the 16S rDNA or rRNA of Ruminococcus gnavus strain CC55_001C, S107-86, S47-18, and/or S107-48. In certain embodiments, the V region of 16S rDNA or rRNA is the V4 region.

In certain embodiments, the lipoglycan-containing antigen or derivative thereof is derived from a bacterial strain from the Blautia genus, wherein the strain has 16S rDNA or rRNA with at least 95%, 96%, 97%, 98%, 99% or including 100% sequence identity to the 16S rDNA or rRNA of Ruminococcus gnavus strain CC55_001C, S107-86, S47-18, and/or S107-48, as described above, over its entire length or at least 95%, 96%, 97%, 98%, 99% or including 100% sequence identity to any single V region of the 16S rDNA or rRNA. In certain embodiments, the lipoglycan-containing antigen or derivative thereof is derived from a bacterial strain from the Blautia genus, wherein the strain has 16S rDNA or rRNA with at least 95%, sequence identity to the 16S rDNA or rRNA of Ruminococcus gnavus strain CC55_001C, S107-86, S47-18, and/or S107-48 over its entire length. In certain embodiments, the lipoglycan-containing antigen or derivative thereof is derived from a bacterial strain from the Blautia genus, wherein the strain has at least 97% sequence identity to any single V region of the 16S rDNA or rRNA of Ruminococcus gnavus strain CC55_001C, S107-86, S47-18, and/or S107-48. In certain embodiments, the V region of 16S rDNA or rRNA is the V4 region. In certain embodiments, the lipoglycan-containing antigen or derivative thereof is derived from a bacterial strain from the Blautia genus, wherein the strain has at least 99% sequence identity to any single V region of the 16S rDNA or rRNA of Ruminococcus gnavus strain CC55_001C, S107-86, S47-18, and/or S107-48. In certain embodiments, the V region of 16S rDNA or rRNA is the V4 region.

In certain embodiments, the lipoglycan-containing antigen or derivative thereof is derived from a bacterial strain from the Ruminococcus gnavus species, wherein the strain has 16S rDNA or rRNA with at least 95%, 96%, 97%, 98%, 99% or including 100% sequence identity to the 16S rDNA or rRNA of Ruminococcus gnavus strain CC55_001C, S107-86, S47-18, and/or S107-48, as described above, over its entire length or at least 95%, 96%, 97%, 98%, 99% or including 100% sequence identity to any single V region of the 16S rDNA or rRNA. In certain embodiments, the lipoglycan-containing antigen or derivative thereof is derived from a bacterial strain from the Ruminococcus gnavus species, wherein the strain has 16S rDNA or rRNA with at least 95%, sequence identity to the 16S rDNA or rRNA of Ruminococcus gnavus strain CC55_001C, S107-86, S47-18, and/or S107-48 over its entire length. In certain embodiments, the lipoglycan-containing antigen or derivative thereof is derived from a bacterial strain from the Ruminococcus gnavus species, wherein the strain has at least 97% sequence identity to any single V region of the 16S rDNA or rRNA of Ruminococcus gnavus strain CC55_001C, S107-86, S47-18, and/or S107-48. In certain embodiments, the V region of 16S rDNA or rRNA is the V4 region. In certain embodiments, the lipoglycan-containing antigen or derivative thereof is derived from a bacterial strain from the Ruminococcus gnavus species, wherein the strain has at least 99% sequence identity to any single V region of the 16S rDNA or rRNA of Ruminococcus gnavus strain CC55_001C, S107-86, S47-18, and/or S107-48. In certain embodiments, the V region of 16S rDNA or rRNA is the V4 region.

In certain embodiments, the bacteria has antigenic gene products other than lipoglycan-containing antigen that leads to systemic lupus erythematosus (SLE), lupus nephritis, incomplete lupus (ILE), undifferentiated connective tissue disease (UCTD), complications of SLE, hidradenitis suppurativa, as well as inflammatory diseases such as, but not limited to, Henoch Schonlein Purpura (HSP), glomerulonephritis (e.g., IgA nephropathy), and IBD (e.g., ulcerative colitis and Crohn's Disease).

In certain embodiments, the lipoglycan-containing antigen comprises a lipoglycan which has a molecular weight between about 20,000 and about 30,000 Daltons. In certain embodiments, the lipoglycan-containing antigen comprises a lipoglycan which has a molecular weight between about 20,000 and about 22,000 Daltons, between about 22,000 and about 24,000 Daltons, between about 21,000 and about 25,000 Daltons, between about 24,000 and about 28,000 Daltons, between about 20,000 and about 26,000 Daltons, between about 26,000 and about 27,000 Daltons, between about 27,000 and about 28,000 Daltons, between about 27,000 and about 28,000 Daltons, between about 28,000 and about 29,000 Daltons, between about 29,000 and about 30,000 Daltons, between about 25,000 and about 27,000 Daltons, between about 26,000 and about 28,000 Daltons, between about 27,000 and about 29,000 Daltons, between about 28,000 and about 30,000 Daltons, between about 25,000 and about 28,000 Daltons, between about 26,000 and about 29,000 Daltons, or between about 27,000 and about 30,000 Daltons. In certain embodiments, the lipoglycan-containing antigen comprises a lipoglycan which has a molecular weight about 20,000 Daltons, about 20,500 Daltons, about 21,000 Daltons, about 21,500 Daltons, about 22,000 Daltons, about 22,500 Daltons, about 23,000 Daltons, about 23,500 Daltons, about 24,000 Daltons, about 24,500 Daltons, about 25,000 Daltons, about 25,500 Daltons, about 26,000 Daltons, about 26,500 Daltons, about 27,000 Daltons, about 27,500 Daltons, about 28,000 Daltons, about 28,500 Daltons, about 29,000 Daltons, about 29,500 Daltons, or about 30,000 Daltons.

In certain embodiments, the lipoglycan-containing antigen comprises a lipoglycan selected from those listed in Table 7 of the present disclosure. In certain embodiments, the isolated antibody or antigen-binding fragment described herein binds to at least one of the lipoglycans listed in Table 7 of the present disclosure. In certain embodiments, the isolated antibody or antigen-binding fragment described herein binds to a plurality of the lipoglycans listed in Table 7 of the present disclosure. In certain embodiments, the isolated antibody or antigen-binding fragment described herein binds to all of the lipoglycans listed in Table 7 of the present disclosure.

In certain embodiments, the isolated antibody or antigen-binding fragment described herein binds an antigen with a lipid anchor. In certain embodiments, the isolated antibody or antigen-binding fragment described herein binds an antigen without a lipid anchor. In certain embodiments, the isolated antibody or antigen-binding fragment described herein binds a purely sugar containing antigen.

In certain embodiments, the lipoglycan-containing antigen comprises a lipoglycan comprising three fatty acids with acyl chain composition of 47:0, one glycerol, eight hexoses, five N-acetyl-hexosamines, and three hexuronic acids (tri-acyl LG 47:0 with a mono-isotopic mass of about 3632), or a derivative thereof.

In certain embodiments, the lipoglycan-containing antigen comprises a lipoglycan comprising two fatty acids with acyl chain composition of 31:0, one glycerol, eight hexoses, five N-acetyl-hexosamines, and three hexuronic acids (di-acyl LG 31:0 with a mono-isotopic mass of about 3394), or a derivative thereof.

In certain embodiments, the lipoglycan-containing antigen comprises a lipoglycan comprising one fatty acid with acyl chain composition of 16:0, one glycerol, eight hexoses, five N-acetyl-hexosamines, and three hexuronic acids (mono-acyl LG 16:0 with a mono-isotopic mass of about 3170), or a derivative thereof.

In certain embodiments, the lipoglycan-containing antigen comprises a lipoglycan comprising one margaric acid with acyl chain composition of 17:0, one glycerol, eight hexoses, five N-acetyl-hexosamines, and three hexuronic acids (mono-acyl LG 17:0 with a mono-isotopic mass of about 3184), or a derivative thereof.

In certain embodiments, bacterial antigen is obtained by treating a culture of bacteria with a lysozyme, a nucleic acid digesting agent (e.g., nucleases), and/or a protease. In certain embodiments, the culture is treated with a protease after treatment with a lysozyme and/or a nucleic acid digesting agent. In certain embodiments, the culture is treated with a protease before treatment with a lysozyme and/or a nucleic acid digesting agent.

In certain embodiments, the bacteria are cultured by ordinary methods known to one of skill in the art. In certain embodiments, the bacteria are cultured in a rich nutrient media. In certain embodiments, the bacteria are cultured in chopped Meat Broth or cultured in brain heart infusion (BHI) broth.

Lysozymes are enzymes that occur naturally in egg white, human tears, saliva, and other body fluids, capable of destroying the cell walls of certain bacteria and thereby acting as a mild antiseptic. Exemplary lysozymes include, but are not limited to, animal-based lysozymes (e.g., human, turkey, chicken, dog, rat), egg white lysozymes (e.g., chickens, ducks, quails, turkeys, and geese), and plant lysozymes.

Nucleic acid fragmentation can be achieved by any method of polynucleotide fragmentation known to those of skill in the art including, but not limited to, nuclease digestion (e.g., restriction enzymes, non-sequence-specific nucleases such as DNase I, micrococcal nuclease, SI nuclease and mung bean nuclease), and physical methods such as shearing and sonication. Isolation is accomplished by any technique that allows for the selective purification of marked fragments from unmarked fragments (e.g., size or affinity separation techniques and/or purification on the basis of a physical property).

Random cleavage can be achieved by enzymatic methods including: a single or a combination of nucleases such as Serratia marcescens, Fragmentase® (New England Biolabs, Ipswich, MA), DNAse I, and Benzonase® (EMD, Gibbstown, NJ), or other types of nucleases. Fragmentase is an endonuclease that generates dsDNA breaks in a time-dependent manner to yield 100 bp-800 bp DNA fragments. Benzonase is genetically engineered endonuclease from Serratia marcescens that can effectively cleave both DNAs and RNAs. Other enzymatic methods include the use of Vvn nuclease alone or Serratia nuclease, or DNase I, or other nuclease in the art such as Shearase™ (Zymo Research, Irvine, CA) or Ion Shear™ (Life Technologies, Grand Island, NY). Nicking enzymes can be used since the DNA is denatured after fragmentation.

Exemplary proteases include, but are not limited to, proteinase K, gelatinase A, gelatinase B, trypsin, trypsin (Arg blocked), trypsin (Lys blocked), clostripain, endoproteinase (e.g., microvillar, Asp-N), chymotrypsin, cyanogen bromide, iodozobenzoate, Myxobacter P., Armillaria, pepsin (e.g., luminal), dipeptidyl peptidase, enteropeptidase, hydrolase, bromelain, ficin, papain, pepsin, plasmin, thermolysin, thrombi, and cathepsins.

In certain embodiments, the bacterial antigen is obtained by a) pelleting a bacterial culture; b) producing a bacterial extract by treating the bacteria with a protein extraction buffer in the presence of a lysozyme, a nuclease, and/or a protease, and a detergent under non-denaturing conditions; c) incubating the mixture; d) removing cell debris (e.g., centrifugation), and using the supernatant as the antigen preparation. In certain embodiments, the bacterial extract is incubated in the presence of a lysozyme, a nuclease, and a protease. In certain embodiments, the nuclease is Serratia marcescens. In certain embodiments, the protease is Proteinase K.

In certain embodiments, the sample may be purified using size exclusion chromatography. In certain embodiments, the sample is enriched for specific characteristic polymers and oligomers and to remove irrelevant components.

In certain embodiments, the bacteria are incubated at 37° C. under anaerobic (75% N₂, 20% CO₂, and 5% H₂) conditions for at least 2, at least 3, at least 4, at least 5, at least 6, or at least 7 days. In certain embodiments, the cells are incubated for about 2 to about 7 days.

In certain embodiments, the lipoglycan-containing antigen is the same as the lipoglycan-containing antigen obtained using a method comprising the steps:
a). culturing Ruminococcus gnavus strain CC55_001C/HM-1056, S107-86, S47-18, or S107-48 at 37° C. under anaerobic conditions for 2-7 days, and
b). producing bacterial extract in the presence of a lysozyme, Serratia marcescens endonuclease, Proteinase K, and a detergent under non-denaturing conditions.

In certain embodiments, the lipoglycan-containing antigen is the same as the lipoglycan-containing antigen obtained using a method comprising the steps:
a). culturing Ruminococcus gnavus strain CC55_001C/HM-1056, S107-86, S47-18, or S107-48 in rich nutrient media at 37° C. under anaerobic conditions (75% N₂, 20% CO₂, and 5% H₂) for 2-7 days,
b). pelleting bacteria by centrifugation,
c). producing a bacterial extract in a protein extraction buffer in the presence of lysozyme, Serratia marcescens endonuclease, and a detergent under non-denaturing conditions,
d). treating the extract obtained in step (c) with Proteinase K,
e). incubating the treated extract obtained in step (d) at 55° C. for about 10 minutes,
f). removing cell debris by centrifugation, and
g). using the supernatant as an antigen preparation.

In certain embodiments, the lipoglycan-containing antigen is the same as the lipoglycan-containing antigen obtained using a method comprising the steps:
a). disrupting Ruminococcus gnavus strain CC55-001C/HM-1056, S107-86, S47-18, or S107-48 cells with a French press,
b). ultracentrifugating to obtain a precipitate and produce an ultracentrifugation supernatant,
c). subjecting the ultracentrifugation supernatant obtained in step (b) to butanol-water extraction and isolating therefrom an aqueous phase,
d). applying the aqueous phase from step (c) to a hydrophobic interaction chromatography matrix, and
e). isolating lipoglycan-containing fractions.

Exemplary antibodies of the present invention are listed in Table 8 herein.

In some embodiments, an antibody or antigen binding fragment described herein comprises a heavy chain complementarity determining region 1 (CDR1), a heavy chain CDR2, a heavy chain CDR3 contained within a heavy chain variable region (VH) comprising the amino acid sequence set forth in SEQ ID NO: 4 or 13. In some embodiments, an antibody or antigen binding fragment described herein comprises a VH comprising an amino acid sequence of SEQ ID NO: 4 or 13, or an amino acid sequence having at least 75%, 80%, 85%, 90%, 91%, 92%, 93%, 94%, 95%, 96%, 97%, 98%, 99%, 95.5%, or 99.9% sequence identity to the amino acid sequence of SEQ ID NO: 4 or 13.

In some embodiments, an antibody or antigen binding fragment described herein comprises a light chain complementarity determining region 1 (CDR1), a light chain CDR2, a light chain CDR3 contained within a light chain variable region (VL) comprising the amino acid sequence set forth in SEQ ID NO: 9 or 15. In some embodiments, an antibody or antigen binding fragment described herein comprises a VL comprising an amino acid sequence of SEQ ID NO: 9 or 15, or an amino acid sequence having at least 75%, 80%, 85%, 90%, 91%, 92%, 93%, 94%, 95%, 96%, 97%, 98%, 99%, 95.5%, or 99.9% sequence identity to the amino acid sequence of SEQ ID NO: 9 or 15.

In some embodiments, the isolated antibody, or antigen-binding fragment thereof, comprises a heavy chain CDR1, a heavy chain CDR2, and a heavy chain CDR3 of a heavy chain variable region (VH) comprising an amino acid sequence of SEQ ID NO: 4 or 13, or a sequence having at least 80%, 85%, 90%, preferably 95% or more, such as 95%, 96%, 97%, 98%, or 99% identity thereto; and a light chain CDR1, a light chain CDR2, and a light chain CDR3 of a light chain variable region (VL) comprising an amino acid sequence of SEQ ID NO: 9 or 15, or a sequence having at least 80%, 85%, 90%, preferably 95% or more, such as 95%, 96%, 97%, 98%, or 99% identity thereto.

In some embodiments, the isolated antibody, or antigen-binding fragment thereof, comprises a heavy chain CDR1, a heavy chain CDR2, and a heavy chain CDR3 of a heavy chain variable region (VH) comprising an amino acid sequence of SEQ ID NO: 4, or a sequence having at least 80%, 85%, 90%, preferably 95% or more, such as 95%, 96%, 97%, 98%, or 99% identity thereto; and a light chain CDR1, a light chain CDR2, and a light chain CDR3 of a light chain variable region (VL) comprising an amino acid sequence of SEQ ID NO: 9, or a sequence having at least 80%, 85%, 90%, preferably 95% or more, such as 95%, 96%, 97%, 98%, or 99% identity thereto.

In some embodiments, the isolated antibody, or antigen-binding fragment thereof, comprises a heavy chain CDR1, a heavy chain CDR2, and a heavy chain CDR3 of a heavy chain variable region (VH) comprising an amino acid sequence of SEQ ID NO: 4; and a light chain CDR1, a light chain CDR2, and a light chain CDR3 of a light chain variable region (VL) comprising an amino acid sequence of SEQ ID NO: 9.

In some embodiments, the isolated antibody, or antigen-binding fragment thereof, comprises a heavy chain CDR1, a heavy chain CDR2, and a heavy chain CDR3 of a heavy chain variable region (VH) comprising an amino acid sequence of SEQ ID NO: 13, or a sequence having at least 80%, 85%, 90%, preferably 95% or more, such as 95%, 96%, 97%, 98%, or 99% identity thereto; and a light chain CDR1, a light chain CDR2, and a light chain CDR3 of a light chain variable region (VL) comprising an amino acid sequence of SEQ ID NO: 15, or a sequence having at least 80%, 85%, 90%, preferably 95% or more, such as 95%, 96%, 97%, 98%, or 99% identity thereto.

In some embodiments, the isolated antibody, or antigen-binding fragment thereof, comprises a heavy chain CDR1, a heavy chain CDR2, and a heavy chain CDR3 of a heavy chain variable region (VH) comprising an amino acid sequence of SEQ ID NO: 13; and a light chain CDR1, a light chain CDR2, and a light chain CDR3 of a light chain variable region (VL) comprising an amino acid sequence of SEQ ID NO: 15.

In some embodiments, the isolated antibody, or antigen-binding fragment thereof, comprises a heavy chain CDR1, a heavy chain CDR2, and a heavy chain CDR3 of a heavy chain variable region (VH) comprising an amino acid sequence of SEQ ID NO: 4, or a sequence having at least 80%, 85%, 90%, preferably 95% or more, such as 95%, 96%, 97%, 98%, or 99% identity thereto; and a light chain CDR1, a light chain CDR2, and a light chain CDR3 of a light chain variable region (VL) comprising an amino acid sequence of SEQ ID NO: 15, or a sequence having at least 80%, 85%, 90%, preferably 95% or more, such as 95%, 96%, 97%, 98%, or 99% identity thereto.

In some embodiments, the isolated antibody, or antigen-binding fragment thereof, comprises a heavy chain CDR1, a heavy chain CDR2, and a heavy chain CDR3 of a heavy chain variable region (VH) comprising an amino acid sequence of SEQ ID NO: 4; and a light chain CDR1, a light chain CDR2, and a light chain CDR3 of a light chain variable region (VL) comprising an amino acid sequence of SEQ ID NO: 15.

In some embodiments, the isolated antibody, or antigen-binding fragment thereof, comprises a heavy chain CDR1, a heavy chain CDR2, and a heavy chain CDR3 of a heavy chain variable region (VH) comprising an amino acid sequence of SEQ ID NO: 13, or a sequence having at least 80%, 85%, 90%, preferably 95% or more, such as 95%, 96%, 97%, 98%, or 99% identity thereto; and a light chain CDR1, a light chain CDR2, and a light chain CDR3 of a light chain variable region (VL) comprising an amino acid sequence of SEQ ID NO: 9, or a sequence having at least 80%, 85%, 90%, preferably 95% or more, such as 95%, 96%, 97%, 98%, or 99% identity thereto.

In some embodiments, the isolated antibody, or antigen-binding fragment thereof, comprises a heavy chain CDR1, a heavy chain CDR2, and a heavy chain CDR3 of a heavy chain variable region (VH) comprising an amino acid sequence of SEQ ID NO: 13; and a light chain CDR1, a light chain CDR2, and a light chain CDR3 of a light chain variable region (VL) comprising an amino acid sequence of SEQ ID NO: 9.

In some embodiments, the heavy chain CDR1 comprises the amino acid sequence set forth in SEQ ID NO: 1, the heavy chain CDR2 comprises the amino acid sequence set forth in SEQ ID NO: 2 or 11, and the heavy chain CDR3 comprises the amino acid sequence set forth in SEQ ID NO: 3 or 12.

In some embodiments, the isolated antibody or antigen-binding fragment comprises a heavy chain CDR1 of SEQ ID NO: 1, and conservative modifications thereof, wherein the isolated antibody or antigen-binding fragment binds a lipoglycan-containing antigen associated with Ruminococcus gnavus strain CC55_001C/HM-1056, S107-86, S47-18, and/or S107-48.

In some embodiments, the isolated antibody or antigen-binding fragment comprises a heavy chain CDR2 of SEQ ID NO: 2 or 11, and conservative modifications thereof, wherein the isolated antibody or antigen-binding fragment binds a lipoglycan-containing antigen associated with Ruminococcus gnavus strain CC55_001C/HM-1056, S107-86, S47-18, and/or S107-48.

In some embodiments, the isolated antibody or antigen-binding fragment comprises a heavy chain CDR3 of SEQ ID NO: 3 or 12, and conservative modifications thereof, wherein the isolated antibody or antigen-binding fragment binds a lipoglycan-containing antigen associated with Ruminococcus gnavus strain CC55_001C/HM-1056, S107-86, S47-18, and/or S107-48.

In some embodiments, the heavy chain CDR1 comprises the amino acid sequence set forth in SEQ ID NO: 1, the heavy chain CDR2 comprises the amino acid sequence set forth in SEQ ID NO: 2, and the heavy chain CDR3 comprises the amino acid sequence set forth in SEQ ID NO: 3.

In some embodiments, the heavy chain CDR1 comprises the amino acid sequence set forth in SEQ ID NO: 1, the heavy chain CDR2 comprises the amino acid sequence set forth in SEQ ID NO: 11, and the heavy chain CDR3 comprises the amino acid sequence set forth in SEQ ID NO: 12.

In some embodiments, the heavy chain CDR1 comprises the amino acid sequence set forth in SEQ ID NO: 1, the heavy chain CDR2 comprises the amino acid sequence set forth in SEQ ID NO: 2, and the heavy chain CDR3 comprises the amino acid sequence set forth in SEQ ID NO: 12.

In some embodiments, the heavy chain CDR1 comprises the amino acid sequence set forth in SEQ ID NO: 1, the heavy chain CDR2 comprises the amino acid sequence set forth in SEQ ID NO: 11, and the heavy chain CDR3 comprises the amino acid sequence set forth in SEQ ID NO: 3.

In some embodiments, the heavy chain CDR1 comprises the amino acid sequence of SEQ ID NO: 21, the heavy chain CDR2 comprises the amino acid sequence of SEQ ID NO: 22, and the heavy chain CDR3 comprises the amino acid sequence of SEQ ID NO: 3.

In some embodiments, the heavy chain CDR1 comprises the amino acid sequence of SEQ ID NO: 21, the heavy chain CDR2 comprises the amino acid sequence of SEQ ID NO: 25, and a heavy chain CDR3 comprises the amino acid sequence of SEQ ID NO: 12.

In some embodiments, the light chain CDR1 comprises the amino acid sequence set forth in SEQ ID NO: 6, the light chain CDR2 comprises the amino acid sequence set forth in SEQ ID NO: 7, and the light chain CDR3 comprises the amino acid sequence set forth in SEQ ID NO: 8.

In some embodiments, the light chain CDR1 comprises the amino acid sequence of SEQ ID NO: 24, the light chain CDR2 comprises the amino acid sequence of KAS, and the light chain CDR3 comprises the amino acid sequence of SEQ ID NO: 8.

In some embodiments, the isolated antibody or antigen-binding fragment comprises a light chain CDR1 of SEQ ID NO: 6, and conservative modifications thereof, wherein the isolated antibody or antigen-binding fragment binds a lipoglycan-containing antigen associated with Ruminococcus gnavus strain CC55_001C/HM-1056, S107-86, 547-18, and/or S107-48.

In some embodiments, the isolated antibody or antigen-binding fragment comprises a light chain CDR2 of SEQ ID NO: 7, and conservative modifications thereof, wherein the isolated antibody or antigen-binding fragment binds a lipoglycan-containing antigen associated with Ruminococcus gnavus strain CC55_001C/HM-1056, S107-86, S47-18, and/or S107-48.

In some embodiments, the isolated antibody or antigen-binding fragment comprises a light chain CDR3 of SEQ ID NO: 8, and conservative modifications thereof, wherein the isolated antibody or antigen-binding fragment binds a lipoglycan-containing antigen associated with Ruminococcus gnavus strain CC55_001C/HM-1056, S107-86, S47-18, and/or S107-48.

In some embodiments, the isolated antibody or antigen-binding fragment comprises the heavy chain CDR1 of SEQ ID NO: 1, the heavy chain CDR2 of SEQ ID NO: 2 or 11, the heavy chain CDR3 of SEQ ID NO: 3 or 12, the light chain CDR1 of SEQ ID NO: 6, the light chain CDR2 of SEQ ID NO: 7 and the light chain CDR3 of SEQ ID NO: 8.

In some embodiments, the isolated antibody or antigen-binding fragment comprises the heavy chain CDR1 of SEQ ID NO: 1, the heavy chain CDR2 of SEQ ID NO: 2, the heavy chain CDR3 of SEQ ID NO: 3, the light chain CDR1 of SEQ ID NO: 6, the light chain CDR2 of SEQ ID NO: 7 and the light chain CDR3 of SEQ ID NO: 8.

In some embodiments, the isolated antibody or antigen-binding fragment comprises the heavy chain CDR1 of SEQ ID NO: 1, the heavy chain CDR2 of SEQ ID NO: 11, the heavy chain CDR3 of SEQ ID NO: 12, the light chain CDR1 of SEQ ID NO: 6, the light chain CDR2 of SEQ ID NO: 7 and the light chain CDR3 of SEQ ID NO: 8.

In some embodiments, the isolated antibody or antigen-binding fragment comprises the heavy chain CDR1 of SEQ ID NO: 1, the heavy chain CDR2 of SEQ ID NO: 2, the heavy chain CDR3 of SEQ ID NO: 12, the light chain CDR1 of SEQ ID NO: 6, the light chain CDR2 of SEQ ID NO: 7 and the light chain CDR3 of SEQ ID NO: 8.

In some embodiments, the isolated antibody or antigen-binding fragment comprises the heavy chain CDR1 of SEQ ID NO: 21, the heavy chain CDR2 of SEQ ID NO: 22, the heavy chain CDR3 of SEQ ID NO: 3, the light chain CDR1 of SEQ ID NO: 24, the light chain CDR2 of KAS and the light chain CDR3 of SEQ ID NO: 8.

In some embodiments, the isolated antibody or antigen-binding fragment comprises the heavy chain CDR1 of SEQ ID NO: 21, the heavy chain CDR2 of SEQ ID NO: 25, the heavy chain CDR3 of SEQ ID NO: 12, the light chain CDR1 of SEQ ID NO: 24, the light chain CDR2 of KAS, and the light chain CDR3 of SEQ ID NO: 8.

In some embodiments, the isolated antibody or antigen-binding fragment comprises the heavy chain CDR1 of SEQ ID NO: 1, the heavy chain CDR2 of SEQ ID NO: 11, the heavy chain CDR3 of SEQ ID NO: 3, the light chain CDR1 of SEQ ID NO: 6, the light chain CDR2 of SEQ ID NO: 7 and the light chain CDR3 of SEQ ID NO: 8.

In some embodiments, the isolated antibody or antigen-binding fragment comprises a VH comprising an amino acid sequence of SEQ ID NO: 4 or 13, or a sequence having at least 80%, 85%, 90%, preferably 95% or more, such as 95%, 96%, 97%, 98%, or 99% identity thereto; and/or a VL comprising an amino acid sequence of SEQ ID NO: 9 or 15, or a sequence having at least 80%, 85%, 90%, preferably 95% or more, such as 95%, 96%, 97%, 98%, or 99% identity thereto.

In some embodiments, isolated antibody or antigen-binding fragment comprises a VH comprising an amino acid sequence of SEQ ID NO: 4 or 13; and/or a VL comprising an amino acid sequence of SEQ ID NO: 9 or 15.

In some embodiments, the isolated antibody or antigen-binding fragment comprises a VH comprising an amino acid sequence of SEQ ID NO: 4, or a sequence having at least 80%, 85%, 90%, preferably 95% or more, such as 95%, 96%, 97%, 98%, or 99% identity thereto; and/or a VL comprising an amino acid sequence of SEQ ID NO: 9, or a sequence having at least 80%, 85%, 90%, preferably 95% or more, such as 95%, 96%, 97%, 98%, or 99% identity thereto.

In some embodiments, isolated antibody or antigen-binding fragment comprises a VH comprising an amino acid sequence of SEQ ID NO: 4; and or a VL comprising an amino acid sequence of SEQ ID NO: 9.

In some embodiments, the isolated antibody or antigen-binding fragment comprises a VH comprising an amino acid sequence of SEQ ID NO: 13, or a sequence having at least 80%, 85%, 90%, preferably 95% or more, such as 95%, 96%, 97%, 98%, or 99% identity thereto; and/or a VL comprising an amino acid sequence of SEQ ID NO: 15, or a sequence having at least 80%, 85%, 90%, preferably 95% or more, such as 95%, 96%, 97%, 98%, or 99% identity thereto.

In some embodiments, the isolated antibody or antigen-binding fragment comprises a VH comprising an amino acid sequence of SEQ ID NO: 13; and or a VL comprising an amino acid sequence of SEQ ID NO: 15.

In some embodiments, the isolated antibody or antigen-binding fragment comprises a VH comprising an amino acid sequence of SEQ ID NO: 4, or a sequence having at least 80%, 85%, 90%, preferably 95% or more, such as 95%, 96%, 97%, 98%, or 99% identity thereto; and/or a VL comprising an amino acid sequence of SEQ ID NO: 15, or a sequence having at least 80%, 85%, 90%, preferably 95% or more, such as 95%, 96%, 97%, 98%, or 99% identity thereto.

In some embodiments, isolated antibody or antigen-binding fragment comprises a VH comprising an amino acid sequence of SEQ ID NO: 4; and or a VL comprising an amino acid sequence of SEQ ID NO: 15.

In some embodiments, the isolated antibody or antigen-binding fragment comprises a VH comprising an amino acid sequence of SEQ ID NO: 13, or a sequence having at least 80%, 85%, 90%, preferably 95% or more, such as 95%, 96%, 97%, 98%, or 99% identity thereto; and/or a VL comprising an amino acid sequence of SEQ ID NO: 9, or a sequence having at least 80%, 85%, 90%, preferably 95% or more, such as 95%, 96%, 97%, 98%, or 99% identity thereto.

In some embodiments, isolated antibody or antigen-binding fragment comprises a VH comprising an amino acid sequence of SEQ ID NO: 13; and or a VL comprising an amino acid sequence of SEQ ID NO: 9.

In certain embodiments, the isolated antibody or antigen-binding fragment comprises one or more amino acid substitutions. In certain embodiments, amino acid substitutions of an antibody or portion thereof are those which: (1) reduce susceptibility to proteolysis, (2) reduce susceptibility to oxidation, (3) alter binding affinity for forming protein complexes, or (4) confer or modify other physicochemical or functional properties. For example, single or multiple amino acid substitutions (preferably conservative amino acid substitutions) may be made in the naturally occurring sequence.

A conservative amino acid substitution should not substantially change the structural characteristics of the parent sequence. Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al., Nature 354:105 (1991), which are each incorporated herein by reference.

As used herein, the twenty naturally occurring amino acids and their abbreviations follow conventional usage. See Immunology—A Synthesis (2^{nd} Edition, E. S. Golub and D. R. Green, Eds., Sinauer Associates, Sunderland, Mass. (1991)), which is incorporated herein by reference.

In some embodiments, the antibody or antigen-binding fragment is recombinant. In certain embodiments, the antibody or antigen-binding fragment is a human antibody, a humanized antibody, a chimeric antibody, a murine antibody, a monoclonal antibody, a single chain antibody, a Fab, a Fab′, a F(ab′)2, a Fv, a scFv, or a nanobody.

In some embodiments, the antibodies described herein are humanized antibodies.

In some embodiments, the antibodies described herein are chimeric antibodies.

In some embodiments, the anti-lipoglycan antibodies disclosed herein, having the heavy chain CDRs disclosed herein, contains framework regions derived from a subclass of germline VH fragment. Such germline VH regions are well known in the art. See, e.g., the IMGT database (imgt.org) or at vbase2.org/vbstat.php. Examples include the IGHV1 subfamily (e.g., IGHV1-2, IGHV1-3, IGHV1-8, IGHV1-18, IGHV1-24, IGHV1-45, IGHV1-46, IGHV1-58, and IGHV1-69), the IGHV2 subfamily (e.g., IGHV2-5, IGHV2-26, and IGHV2-70), the IGHV3 subfamily (e.g., IGHV3-7, IGHV3-9, IGHV3-11, IGHV31-13, IGHV3-15, IGHV3-20, IGHV3-21, IGHV3-23, IGHV3-30, IGHV3-33, IGHV3-43, IGHV3-48, IGHV3-49, IGHV3-53, IGHV3-64, IGHV3-66, IGHV3-72, and IGHV3-73, IGHV3-74), the IGHV4 subfamily (e.g., IGHV4-4, IGHV4-28, IGHV4-31, IGHV4-34, IGHV4-39, IGHV44-59, IGHV4-61, and IGHV4-B), the IGHV subfamily (e.g., IGHV5-51, or IGHV6-1), and the IGHV7 subfamily (e.g., IGHV7-4-1).

Alternatively, or in addition, in some embodiments, the anti-lipoglycan antibody, having the light chain CDRs disclosed herein, contains framework regions derived from a germline Vκ fragment. Examples include an IGKV1 framework (e.g., IGKV1-05, IGKV1-12, IGKV1-27, IGKV1-33, or IGKV1-39), an IGKV2 framework (e.g., IGKV2-28), an IGKV3 framework (e.g., IGKV3-11, IGKV3-15, or IGKV3-20), and an IGKV4 framework (e.g., IGKV4-1). In other instances, the anti-Lipoglycan antibody comprises a light chain variable region that contains a framework derived from a germline V) fragment. Examples include an IGλ1 framework (e.g., IGλV1-36, IGλV1-40, IGλV1-44, IGλV1-47, IGλV1-51), an IGλ2 framework (e.g., IGλV2-8, IGλV2-11, IGλV2-14, IGλV2-18, IGλV2-23), an IGλ3 framework (e.g., IGλV3-1, IGλV3-9, IGλV3-10, IGλV3-12, IGλV3-16, IGλV3-19, IGλV3-21, IGλV3-25, IGλV3-27), an IGλ4 framework (e.g., IGλV4-3, IGλV4-60, IGλV4-69), an IGλ5 framework (e.g., IGλV5-39, IGλV5-45), an IGλ6 framework (e.g., IGλV6-57), an IGλ7 framework (e.g., IGλV7-43, IGλV7-46), an IGλ8 framework (e.g., IGλV8-61), an IGλ9 framework (e.g., IGλV9-49), or an IGλ10 framework (e.g., IGλ10-54).

In some embodiments, the described antibodies may be used to develop new forms that retain the lipoglycan binding specificity through, e.g., the application of phage-display technology.

In some embodiments, the anti-lipoglycan antibody disclosed herein is a functional variant of the reference antibody mAb 33.2.2 or mAb 34.2.2 as set forth in Table 8. A functional variant can be structurally similar as the reference antibody (e.g., comprising the limited number of amino acid residue variations in one or more of the heavy chain and/or light chain CDRs as mAb 33.2.2 or mAb 34.2.2 as disclosed herein, or the sequence identity relative to the heavy chain and/or light chain CDRs of mAb 33.2.2 or mAb 34.2.2, or the VH and/or VL of mAb 33.2.2 or mAb 34.2.2 as disclosed herein) with substantially similar binding affinity (e.g., having a KD value in the same order) to the described bacterial lipoglycan.

In some embodiments, the anti-lipoglycan antibody disclosed herein competes for binding to a lipoglycan-containing antigen with the reference antibody mAb 33.2.2 or mAb 34.2.2 as set forth in Table 8.

In some embodiments, the anti-lipoglycan antibody disclosed herein binds to the same epitope on a lipoglycan-containing antigen with the reference antibody mAb 33.2.2 or mAb 34.2.2 as set forth in Table 8.

In some embodiments, the anti-lipoglycan antibody as described herein can bind and inhibit growth and/or activity of one or more bacterial strains associated with the lipoglycan-containing antigen. In some embodiments, the anti-lipoglycan antibody as described herein can bind and inhibit growth and/or activity of one or more bacterial strains associated with the lipoglycan-containing antigen by at least 20% (e.g., 31%, 35%, 40%, 45%, 50%, 60%, 70%, 80%, 90%, 95% or greater, including any increment therein).

In some embodiments, the anti-lipoglycan antibody as described herein can bind and reduce the amount of the described target lipoglycan or the bacterial strain associated with the target lipoglycan in a subject infected with the bacterial strain. In some embodiments, the anti-lipoglycan antibody as described herein can bind and reduce the amount of the described target lipoglycan or the bacterial strain associated with the target lipoglycan in a subject by at least 20% (e.g., 31%, 35%, 40%, 45%, 50%, 60%, 70%, 80%, 90%, 95% or greater, including any increment therein).

In some embodiments, an anti-lipoglycan antibody or antigen-binding fragment as described herein has a suitable binding affinity for the target antigen (e.g., lipoglycan) or antigenic epitopes thereof. As used herein, “binding affinity” refers to the apparent association constant or K_{A}. The K_{A} is the reciprocal of the dissociation constant (K_{D}). The anti-lipoglycan antibody described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased Kr. Binding affinity (or binding specificity) can be determined by a variety of methods including equilibrium dialysis, equilibrium binding, gel filtration, ELISA, surface plasmon resonance, bead-based assay, radioimmunoassay, or spectroscopy (e.g., using a fluorescence assay). In certain embodiments, the bacterial antigen, or derivative thereof, is coated onto a bead or onto the surface of an ELISA plate or other solid phase used for measurement.

These techniques can be used to measure the concentration of bound antibody or antigen-binding fragment as a function of target antigen concentration. Under certain conditions, the fractional concentration of bound antibody or antigen-binding fragment ([Bound]/[Total]) is generally related to the concentration of total target antigen ([Target]) by the following equation:

[Bound]/[Total]=[Target]/(K_{D}+[Target])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay. In some cases, the in vitro binding assay is indicative of in vivo activity. In other cases, the in vitro binding assay is not necessarily indicative of in vivo activity. In some cases, tight binding is beneficial, but in other cases tight binding is not as desirable in the in vivo setting, and an antibody with lower binding affinity is more desirable.

In some embodiments, the heavy chain of any of any of the anti-lipoglycan antibodies as described herein further comprise a heavy chain constant region (CH) or a portion thereof (e.g., CH1, CH2, CH3, or a combination thereof). The heavy chain constant region can be of any suitable origin, e.g., human, mouse, rat, or rabbit. In some embodiments, the heavy chain constant region is from an IgD, IgE, IgG, IgA, or IgM class, or sub-class thereof. In some embodiments, the heavy chain constant region is from a human IgG (a gamma heavy chain) or any IgG subfamily as described herein. In some embodiments, the heavy chain constant region is from an IgG1, IgG2, IgG3, or IgG4 subclass.

In some embodiments, the heavy chain constant region of the antibodies described herein comprise a single domain (e.g., CH1, CH2, or CH3) or a combination of any of the single domains, of a constant region. In some embodiments, the light chain constant region of the antibodies described herein comprise a single domain (e.g., CL), of a constant region.

In some embodiments, the anti-lipoglycan antibody or antigen-binding fragment described herein is of an IgG1 subclass.

In some embodiments, the anti-lipoglycan antibody or antigen-binding fragment described herein is of an IgG2 subclass.

In one aspect, the present disclosure provides a polynucleotide encoding an anti-lipoglycan antibody or antigen-binding fragment as described herein. The isolated polynucleotides capable of encoding the variable domain segments provided herein may be included on the same, or different, vectors to produce antibodies or antigen-binding fragments. Exemplary polynucleotide sequences that may encode the VH or VL of an anti-lipoglycan antibody described herein, or an antigen-binding fragment thereof, are shown in Table 8.

In some embodiments, an isolated polynucleotide encoding an anti-lipoglycan antibody or antigen-binding fragment comprises a VH-encoding nucleotide sequence of SEQ ID NO: 5 or 14, or a sequence having at least 80%, 85%, 90%, preferably 95% or more, such as 95%, 96%, 97%, 98%, or 99% identity thereto; and/or a VL-encoding nucleotide sequence of SEQ ID NO: 10 or 16, or a sequence having at least 80%, 85%, 90%, preferably 95% or more, such as 95%, 96%, 97%, 98%, or 99% identity thereto.

In some embodiments, an isolated polynucleotide encoding an anti-lipoglycan antibody or antigen-binding fragment comprises a VH-encoding nucleotide sequence of SEQ ID NO: 5 or 14; and/or a VL-encoding nucleotide sequence of SEQ ID NO: 10 or 16.

In some embodiments, an isolated polynucleotide encoding an anti-lipoglycan antibody or antigen-binding fragment comprises a VH-encoding nucleotide sequence of SEQ ID NO: 5, or a sequence having at least 80%, 85%, 90%, preferably 95% or more, such as 95%, 96%, 97%, 98%, or 99% identity thereto; and/or a VL-encoding nucleotide sequence of SEQ ID NO: 10, or a sequence having at least 80%, 85%, 90%, preferably 95% or more, such as 95%, 96%, 97%, 98%, or 99% identity thereto.

In some embodiments, an isolated polynucleotide encoding an anti-lipoglycan antibody or antigen-binding fragment comprises a VH-encoding nucleotide sequence of SEQ ID NO: 5; and a VL-encoding nucleotide sequence of SEQ ID NO: 10.

In some embodiments, an isolated polynucleotide encoding an anti-lipoglycan antibody or antigen-binding fragment comprises a VH-encoding nucleotide sequence of SEQ ID NO: 14, or a sequence having at least 80%, 85%, 90%, preferably 95% or more, such as 95%, 96%, 97%, 98%, or 99% identity thereto; and/or a VL-encoding nucleotide sequence of SEQ ID NO: 16, or a sequence having at least 80%, 85%, 90%, preferably 95% or more, such as 95%, 96%, 97%, 98%, or 99% identity thereto.

In some embodiments, an isolated polynucleotide encoding an anti-lipoglycan antibody or antigen-binding fragment comprises a VH-encoding nucleotide sequence of SEQ ID NO: 14; and a VL-encoding nucleotide sequence of SEQ ID NO: 16.

In some embodiments, an isolated polynucleotide encoding an anti-lipoglycan antibody or antigen-binding fragment comprises a VH-encoding nucleotide sequence of SEQ ID NO: 5, or a sequence having at least 80%, 85%, 90%, preferably 95% or more, such as 95%, 96%, 97%, 98%, or 99% identity thereto; and/or a VL-encoding nucleotide sequence of SEQ ID NO: 16, or a sequence having at least 80%, 85%, 90%, preferably 95% or more, such as 95%, 96%, 97%, 98%, or 99% identity thereto.

In some embodiments, an isolated polynucleotide encoding an anti-lipoglycan antibody or antigen-binding fragment comprises a VH-encoding nucleotide sequence of SEQ ID NO: 5; and a VL-encoding nucleotide sequence of SEQ ID NO: 16.

In some embodiments, an isolated polynucleotide encoding an anti-lipoglycan antibody or antigen-binding fragment comprises a VH-encoding nucleotide sequence of SEQ ID NO: 14, or a sequence having at least 80%, 85%, 90%, preferably 95% or more, such as 95%, 96%, 97%, 98%, or 99% identity thereto; and/or a VL-encoding nucleotide sequence of SEQ ID NO: 10, or a sequence having at least 80%, 85%, 90%, preferably 95% or more, such as 95%, 96%, 97%, 98%, or 99% identity thereto.

In some embodiments, an isolated polynucleotide encoding an anti-lipoglycan antibody or antigen-binding fragment comprises a VH-encoding nucleotide sequence of SEQ ID NO: 14; and a VL-encoding nucleotide sequence of SEQ ID NO: 10.

In some embodiments, the polynucleotide is a DNA molecule or a derivative thereof.

In some embodiments, the polynucleotide is an RNA molecule (e.g., mRNA) or a derivative thereof.

Also provided are vectors comprising the polynucleotides described herein. The vectors can be expression vectors. Recombinant expression vectors containing a sequence encoding a polypeptide of interest are thus contemplated as within the scope of this disclosure. The expression vector may contain one or more additional sequences such as but not limited to regulatory sequences (e.g., promoter, enhancer), a selection marker, and a polyadenylation signal. Vectors for transforming a wide variety of host cells are well known and include, but are not limited to, plasmids, phagemids, cosmids, baculoviruses, bacmids, bacterial artificial chromosomes (BACs), yeast artificial chromosomes (YACs), as well as other bacterial, yeast and viral vectors. In some embodiments, the vector is a viral vector.

Recombinant expression vectors within the scope of the description include synthetic, genomic, or cDNA-derived nucleic acid fragments that encode at least one recombinant protein which may be operably linked to suitable regulatory elements. Such regulatory elements may include a transcriptional promoter, sequences encoding suitable mRNA ribosomal binding sites, and sequences that control the termination of transcription and translation. Expression vectors, especially mammalian expression vectors, may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

Vectors described herein may contain one or more Internal Ribosome Entry Site(s) (IRES). Inclusion of an IRES sequence into fusion vectors may be beneficial for enhancing expression of some proteins. In some embodiments the vector system will include one or more polyadenylation sites (e.g., SV40), which may be upstream or downstream of any of the aforementioned nucleic acid sequences. Vector components may be contiguously linked, or arranged in a manner that provides optimal spacing for expressing the gene products (i.e., by the introduction of “spacer” nucleotides between the ORFs), or positioned in another way. Regulatory elements, such as the IRES motif, may also be arranged to provide optimal spacing for expression.

The vectors may comprise selection markers, which are well known in the art. Selection markers include positive and negative selection markers, for example, antibiotic resistance genes (e.g., neomycin resistance gene, a hygromycin resistance gene, a kanamycin resistance gene, a tetracycline resistance gene, a penicillin resistance gene, a puromycin resistance gene, a blasticidin resistance gene), glutamate synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

In some embodiments, the vectors are suitable for expression within the human gastrointestinal tract in a manner that high levels of the anti-lipoglycan antibody would be directed to mucosal surface and/or gut lumen. As non-limiting examples, vectors that can be used to target gastrointestinal tract include Moloney murine leukemia viruses (MLV), Moloney murine leukemia viruses pseudotyped with vesicular stomatitis virus G protein (MLV-VSV-G), murine stem cell viruses (MSCV), lentiviruses, lentiviruses pseudotyped with vesicular stomatitis virus G protein (LV-VSV-G), adenoviruses (e.g., Ad5, Ad41), adeno-associated viruses (such as AAV1, AAV2, AAV5, AAV8, AAV9, AAV10, AAV6), and variants and derivatives thereof. Additional suitable vectors include those described in Buckinx and Timmermans, Histochem Cell Biol (2016) 146:709-720, which is incorporated herein by reference in its entirety.

In some embodiments, bacteriophages may be engineered to incorporate a nucleotide sequence encoding an antibody described herein into the genetic material of the phage so that the antibody may be exposed on the surface of the phage. The phage would then preferentially infect these lipoglycan-bearing bacteria, and destroy the bacteria or remove or modify genes in the bacteria responsible for production of the lipoglycan or other genes that convey pathogenic properties to the bacteria.

The vectors described herein may be used to transform various cells with the genes encoding the described antibodies or antigen-binding fragments. For example, the vectors may be used to generate lipoglycan-specific antibody or antigen-binding fragment-producing cells. Thus, in another aspect is provided host cells transformed with vectors comprising a nucleic acid sequence encoding an antibody or antigen-binding fragment thereof that specifically binds a lipoglycan-containing antigen associated with Ruminococcus gnavus strain CC55_001C/HM-1056, S107-86, S47-18, and/or S107-48, such as the antibodies or antigen-binding fragments described and exemplified herein.

Numerous techniques are known in the art for the introduction of foreign genes into cells and may be used to construct the recombinant cells for purposes of carrying out the described methods, in accordance with the various embodiments described and exemplified herein. The technique used should provide for the stable transfer of the heterologous gene sequence to the host cell, such that the heterologous gene sequence is heritable and expressible by the cell progeny, and so that the necessary development and physiological functions of the recipient cells are not disrupted. Techniques which may be used include but are not limited to chromosome transfer (e.g., cell fusion, chromosome mediated gene transfer, micro cell mediated gene transfer), physical methods (e.g., transfection, spheroplast fusion, microinjection, electroporation, liposome carrier), viral vector transfer (e.g., recombinant DNA viruses, recombinant RNA viruses) and the like (described in Cline, 29 Pharmac. Ther. 69-92 (1985)). Calcium phosphate precipitation and polyethylene glycol (PEG)-induced fusion of bacterial protoplasts with mammalian cells may also be used to transform cells.

In some embodiments, the present disclosure also provides an antibody-drug conjugate comprising the isolated antibody or antigen-binding fragment described herein conjugated to a second moiety. For example, the second moiety can be a bacterial toxin or antibiotic. Such antibody-drug conjugates would be preferentially delivered to the lipoglycan-bearing bacteria and reduce bacterial proliferation or cause bacterial death.

### Preparation of Antibodies or Antigen-Binding Fragments

Antibodies or antigen-binding fragments capable of binding bacterial lipoglycan as described herein can be made by any method known in the art, including but not limited to, recombinant technology.

For example, nucleic acids encoding the heavy and light chain of an anti-lipoglycan antibody as described herein can be cloned into one expression vector, each nucleotide sequence being in operable linkage to a suitable promoter. In one example, each of the nucleotide sequences encoding the heavy chain and light chain is in operable linkage to a distinct promoter.

Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987); Gossen and Bujard (1992); M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-lipoglycan antibody, as also described herein. The recombinant expression vector can be introduced into a suitable host cell (e.g., a dhfr− CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be co-incubated under suitable conditions allowing for the formation of the antibody.

In one example, two recombinant expression vectors are provided, one encoding the heavy chain of the anti-lipoglycan antibody and the other encoding the light chain of the anti-lipoglycan antibody. Both of the two recombinant expression vectors can be introduced into a suitable host cell (e.g., dhfr− CHO cell) by a conventional method, e.g., calcium phosphate-mediated transfection. Alternatively, each of the expression vectors can be introduced into suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-lipoglycan antibody as described herein, vectors (e.g., expression vectors) containing such; and host cells comprising the vectors are within the scope of the present disclosure.

In some embodiments, the antibody or antigen-binding fragment described herein are isolated from an animal immunized with a lipoglycan-containing antigen (e.g., lipoglycan-containing antigens associated with Ruminococcus gnavus strain CC55_001C/HM-1056, S107-86, S47-18, and/or S107-48), or colonized with a Ruminococcus gnavus strain (e.g., CC55_001C/HM-1056, S107-86, S47-18, and/or S107-48) in the gastrointestinal tract, or genetically modified to produce the antibody or antigen-binding fragment. The animal may be genetically modified with the polynucleotide or the vector described herein.

In some embodiments, the animal is a dairy animal, such as, but not limited to, a goat, a cow, a buffalo, a sheep, or a camel. In such cases, the antibody or antigen-binding fragment may be isolated from milk produced by the dairy animal.

Anti-lipoglycan antibodies prepared as described herein can be characterized using methods known in the art, whereby reduction, amelioration, or neutralization of biological activity associated with the target lipoglycan or a bacterial strain expressing the lipoglycan is detected and/or measured. For example, in some embodiments, an ELISA-type assay is suitable for qualitative or quantitative measurement of binding of the antibody to the target lipoglycan.

In some embodiments, the bioactivity of an anti-lipoglycan antibody can verified by incubating a candidate antibody with a bacterial strain expressing the lipoglycan, and monitoring inhibition of growth and/or activity of the bacterial strain.

### Anti-Bacterial and Other Therapeutic Methods of the Invention

In one aspect, the invention provides a method for treating (including preventing) a disease or disorder caused by a lipoglycan-containing antigen and/or a bacterial strain associated with the lipoglycan-containing antigen in a subject in need thereof. The method may comprise administering to the subject an anti-lipoglycan antibody or antigen-binding fragment described herein, or a polynucleotide or vector encoding the antibody or antigen-binding fragment, or pharmaceutical compositions thereof. In certain embodiments, the growth is inhibited to the extent that the target bacterial strains are removed from the microbiota (i.e., reduced or ablated).

In some embodiments, the disease or disorder is systemic lupus erythematosus (SLE), lupus nephritis, hidradenitis suppurativa, inflammatory bowel disease (IBD), incomplete lupus (ILE), undifferentiated connective tissue disease (UCTD), complications of SLE, IgA nephropathy, Henoch Schonlein Purpura (HSP), and other types of glomerulonephritis.

In some embodiments, the disease or disorder is SLE, lupus nephritis, or inflammatory diseases such as, but not limited to, glomerulonephritis (e.g., IgA nephropathy) and inflammatory bowel disease (IBD) (e.g., ulcerative colitis and Crohn's disease).

In certain embodiments, lupus nephritis is proliferative lupus nephritis or membranous lupus nephritis. In certain embodiments, lupus nephritis is membranoproliferative lupus nephritis. In certain embodiments, lupus nephritis is mesangial glomerulonephritis.

In certain embodiments, the methods involve diagnosis and/or treating or preventing complications involved with lupus. Complication of lupus involves, for example but not limitation, the pulmonary system, central nervous system, cardiovascular system, skin disease, joint disease, musculoskeletal disease, depressed red cell levels, depressed white cell levels, depressed platelets, immunosuppression, severe infection, or any combination thereof.

Glomerulonephritis is a group of diseases that injure the part of the kidney that filters blood (called glomeruli). In certain embodiments, glomerulonephritis can be acute or chronic. The range of glomerulonephritis is disclosed in ICD-10. International Statistical Classification of Diseases and Related Health Problems Tenth Revision. Second Edition, which is incorporated herein by reference in its entirety and for all purposes as if fully set forth herein. In certain embodiments, glomerulonephritis entails an inflammation of either the glomeruli or the small blood vessels of the kidneys. In certain embodiments, glomerulonephritis does not entail inflammation. In certain embodiments, the glomerulonephritis disorder can be caused by certain infections (e.g., bacterial, viral or parasitic pathogens), drugs, systemic disorders (e.g., SLE, vasculitis), or diabetes. In certain embodiments, glomerulonephritis can be IgA nephropathy.

In certain embodiments, the glomerulonephritis is associated with lupus. The current classification scheme for glomerulonephritis in SLE patients reflects the understanding of the pathogenesis of the various forms of Lupus nephritis, but clinicopathologic studies have revealed the need for improved categorization and terminology. Based on the 1982 classification published under the auspices of the World Health Organization (WHO) and subsequent clinicopathologic data, class I and II refers to purely mesangial involvement (I, mesangial immune deposits without mesangial hypercellularity; II, mesangial immune deposits with mesangial hypercellularity); class III for focal glomerulonephritis (involving <50% of total number of glomeruli) with subdivisions for active and sclerotic lesions; class IV for diffuse glomerulonephritis (involving > or =50% of total number of glomeruli) either with segmental (class IV-S) or global (class IV-G) involvement, and also with subdivisions for active and sclerotic lesions; class V for membranous lupus nephritis; and class VI for advanced sclerosing lesions]. Combinations of membranous and proliferative glomerulonephritis (i.e., class III and V or class IV and V) are also reported (Weening J J, et al. The classification of glomerulonephritis in systemic lupus erythematosus revisited. J Am Soc Nephrol 2004; 15:241-50).

IgA nephropathy (a.k.a. IgA nephritis, Berger disease, or synpharyngitic glomerulonephritis) occurs when immunoglobulin A (IgA) antibody deposits lodge in the kidneys. In certain embodiments, IgA nephropathy is a kidney disease associated with inflammation of the glomeruli of the kidney and/or IgA deposits within the kidneys. In certain embodiments, IgA nephropathy includes related disorders such as, but not limited to, Henoch Schonlein Purpura (HSP).

IBD is an inflammatory condition of the colon and/or small intestine. Increases in the abundance of Ruminococcus gnavus in the intestine have been reported to occur in some subjects, and clinical subsets of Inflammatory Bowel Disease, which include ulcerative colitis and Crohns disease (Willing B P, et al. Gastroenterology 2010; 139:1844; Png C W, et al. Am J Gastroenterol 2010; 105:2420-8; Joossens M, Huys G, Cnockaert M, et al. Dysbiosis of the faecal microbiota in patients with Crohn's disease and their unaffected relatives. Gut 2011; 60:631-7). In particular, Ileal Crohns Disease has been associated with increases in R. gnavus, although the contributory mechanisms of R. gnavus were not explored. In certain embodiments, the IBD is an inflammatory condition of the colon, small intestine, large intestine, mouth, esophagus, stomach, anus, or rectum. In certain embodiments, the IBD is Crohn's disease, ulcerative colitis, microscopic colitis (e.g., collagenous colitis, lymphocytic colitis), diversion colitis, Behcet's disease, or indeterminate colitis. In certain embodiments, the IBD is Crohn's disease or ulcerative colitis.

Hidradenitis suppurativa (HS) is a chronic inflammatory condition that presents itself on the skin and is characterized by the formation of nodules, abscesses and fistula at intertriginous sites. It is estimated that up to 4 in 100 people suffer from HS as of 2020. Research surrounding the skin-gut axis has been emerging to understand the potential contributory factor of the pathogenesis of Hidradenitis suppurativa. In a study consisting of 59HS patient samples were collected to study the state of the patients' fecal, nasal and skin microbiotas. Results showed that the microbiome alpha diversity of the fecal, nasal and skin samples in HS patients was significantly lower when compared to 50 healthy controls (30 for fecal samples and 20 for nasal and skin swabs). Ruminococcus gnavus was more abundant in the fecal microbiome and art overabundance of Finegoldia magna was seen in the skin samples of HS patients compared to healthy controls.

In one embodiment of any of the methods of the invention, the antibody or composition described herein is administered to the subject by a route selected from the group consisting of oral, nasal, rectal (e.g., by enema), mucosal, sublingual, and via naso/oro-gastric gavage. In one embodiment, the antibody or composition described herein is administered directly to the GI of the subject.

In some embodiments, the antibody or composition described herein inhibits growth and/or activity of one or more strains of bacteria from Ruminococcus gnavus. In certain embodiments, the antibody or composition inhibits growth and/or activity of at least 2, 3, 4, 5, 6, 7, 8, 9, 10, 20, 50 or more strains from Ruminococcus gnavus. In certain embodiments, the antibody or composition described herein inhibits growth and/or activity of the Ruminococcus gnavus strain CC55_001C, S107-86, S47-18, and/or S107-48.

In some embodiments of any of the above methods involving administration of an antibody or composition described herein that inhibits growth and/or activity of one or more strains of bacteria of Ruminococcus gnavus or a closely related OTUs which are independently characterized by, e.g., at least 95%, 96%, 97%, 98%, 99% or including 100% sequence identity to 16S rDNA or rRNA sequences of the bacteria from Ruminococcus gnavus strain CC55_001C, S107-86, S47-18, and/or S107-48. In another embodiment, the OTUs may be characterized by one or more of the variable regions of the 16S rDNA or rRNA sequence (V1-V9). These regions in bacteria are defined by nucleotides 69-99, 137-242, 433-497, 576-682, 822-879, 986-1043, 1117-1173, 1243-1294 and 1435-1465, respectively, using numbering based on the E. coli system of nomenclature. (See, e.g., Brosius et al., Complete nucleotide sequence of a 16S ribosomal RNA gene from Escherichia coli, PNAS 75(10):4801-4805 (1978)). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU.

In some embodiments, the antibody or composition described herein is administered to the subject in an effective amount sufficient to inhibit the biosynthesis of lipoglycan-containing antigen or a derivative thereof or of a bacterial antigen as described above. In some embodiments, the composition is administered to the subject in an effective amount sufficient to increase the removal of lipoglycan-containing antigen or derivative thereof or of a bacterial antigen from the body or to block the immunologic and biologic effects of the lipoglycan-containing antigen, derivative thereof or of a bacterial antigen.

In certain embodiments, the method comprises administering to the subject an effective amount of an antibody or composition described herein to inhibit growth and/or activity of one or more strains of bacteria from the phylum of Firmicutes in the GI microbiota of the subject. In certain embodiments, the antibody or composition inhibits growth and/or activity of at least 2, 3, 4, 5, 6, 7, 8, 9, 10, 20, 50 or more strains from this taxon.

In certain embodiments, the method comprises administering to the subject an effective amount of an antibody or composition to inhibit growth and/or activity of one or more strains of bacteria from the class of Clostridia in the GI microbiota of the subject. In certain embodiments, the antibody or composition inhibits growth and/or activity of at least 2, 3, 4, 5, 6, 7, 8, 9, 10, 20, 50 or more strains from this taxon.

In certain embodiments, the method comprises administering to the subject an effective amount of an antibody or composition to inhibit growth and/or activity of one or more strains of bacteria from the order of Clostridiales in the GI microbiota of the subject. In certain embodiments, the antibody or composition inhibits growth and/or activity of at least 2, 3, 4, 5, 6, 7, 8, 9, 10, 20, 50 or more strains from this taxon.

In certain embodiments, the method comprises administering to the subject an effective amount of an antibody or composition to inhibit growth and/or activity of one or more strains of bacteria from the family of Lachnospiraceae or Ruminococcaceae in the GI microbiota of the subject. In certain embodiments, the antibody or composition inhibits growth and/or activity of at least 2, 3, 4, 5, 6, 7, 8, 9, 10, 20, 50 or more strains from these taxa.

In certain embodiments, the method comprises administering to the subject an effective amount of an antibody or composition to inhibit growth and/or activity of one or more strains of bacteria from the genus Blautia or Ruminococcus in the GI microbiota of the subject. In certain embodiments, the antibody or composition inhibits growth and/or activity of at least 2, 3, 4, 5, 6, 7, 8, 9, 10, 20, 50 or more strains from these taxa.

In certain embodiments, the method comprises administering to the subject an effective amount of an antibody or composition to inhibit growth and/or activity of one or more strains of bacteria from the species Ruminococcus gnavus in the GI microbiota of the subject. In certain embodiments, the antibody or composition inhibits growth and/or activity of at least 2, 3, 4, 5, 6, 7, 8, 9, 10, 20, 50 or more strains from this taxon. In certain embodiments, the antibody or composition inhibits the immunologic activity of the strain associated with the bacterial antigen. In certain embodiments, the antibody or composition inhibits the expression of the bacterial antigen. In certain embodiments, the antibody or composition reduces the content of the bacterial antigen within the bacteria. In certain embodiments, the antibody or composition reduces the content of the bacterial antigen within the GI tract. In certain embodiments, the antibody or composition reduces the content of the bacterial antigen within the systemic circulation.

In one embodiment of any of the above methods of the invention, the antibody or composition that inhibits growth and/or activity of one or more strains of bacteria is administered in a therapeutically effective amount. The dosages of the antibody or composition administered in the methods of the invention will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to reduce or eradicate colonization.

In some instances, the anti-lipoglycan antibody as disclosed herein can be administered to a subject at a suitable dose, for example, about 0.5 to about 32 mg/kg. Examples include 0.5 mg/kg to 1 mg/kg, 1 mg/kg to 2 mg/kg, 2 mg/kg to 3 mg/kg, 3 mg/kg to 4 mg/kg, 4 mg/kg to 8 mg/kg, 8 mg/kg to 12 mg/kg, 12 mg/kg to 16 mg/kg, 16 mg/kg to 20 mg/kg, 20 mg/kg to 24 mg/kg, 24 mg/kg to 28 mg/kg, or 28 mg/kg to 32 mg/kg (e.g., 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, or 32 mg/kg) or any incremental doses within these ranges. In some embodiments, the antibody is administered at a dose of about 0.5 about mg/kg to 1 mg/kg, about 1 mg/kg to 2 mg/kg, about 2 mg/kg to 4 mg/kg, about 4 mg/kg to 8 mg/kg, about 8 mg/kg to 12 mg/kg, about 12 mg/kg to 16 mg/kg, about 16 mg/kg to 20 mg/kg, about 20 mg/kg to 24 mg/kg, about 24 mg/kg to 28 mg/kg, or about 28 mg/kg to 32 mg/kg (e.g., about 0.5 mg/kg, about 1 mg/kg, about 2 mg/kg, about 3 mg/kg, about 4 mg/kg, about 5 mg/kg, about 6 mg/kg, 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, or about 32 mg/kg) or any incremental doses within these ranges.

In some embodiments, the antibody or composition that inhibits growth and/or activity of one or more strains of bacteria according to any of the above methods can be natural products that inhibit microbial growth. In certain embodiments, the antibody or composition that inhibits growth and/or activity of one or more strains of bacteria according to any of the above methods can be bacteria that is conditionally lethal engineered bacteria (e.g., H. pylori, E. coli, etc). In certain embodiments, the antibody or composition that inhibits growth and/or activity of one or more strains of bacteria according to any of the above methods can be genetically engineered commensals strains of microorganisms.

In some embodiments, suppressing growth or activity of at least one bacterial species in the microbiota according to any of the above methods involving such suppression can be achieved, e.g., by administering an antibiotic. In one specific embodiment, the antibiotic is administered in a therapeutic dose. In another specific embodiment, the antibiotic is administered in a sub-therapeutic dose. Non-limiting examples of antibiotics useful in the methods of the invention include beta-lactams (e.g., Penicillin VK, Penicillin G, Amoxicillin trihydrate), nitroimidazoles, macrolides (e.g., Tylosin tartrate, Erythromycin, Azithromycin, and Clarithromycin), tetracyclines, glycopeptides (e.g., Vancomycin), and fluoroquinolones. In one specific embodiment, the method comprises administering Penicillin VK or Penicillin G at 1 mg/kg body weight per day for at least four weeks of life. In another specific embodiment, the method comprises administering Amoxicillin trihydrate at 25 mg/kg body weight per day for 1 to 3 treatments each lasting 3 to 5 days. In yet another specific embodiment, the method comprises administering Tylosin tartrate at 50 mg/kg body weight per day for 1 to 3 treatments each lasting 3 to 5 days.

In certain embodiments, the method comprises administering to the subject an effective amount of an antibody or composition, wherein the antibody or composition results in a decrease in the level of the antibodies to a lipoglycan-containing antigen or a derivative thereof or to a bacterial antigen as described above. In certain embodiments, the antibody of composition binds and neutralizes the lipoglycan-containing antigen or derivative thereof or the bacterial antigen or aids in the clearance of lipoglycan-containing antigen or derivative thereof or the bacterial antigen from the GI or circulation.

In certain embodiments, the antibody is an antibody or a functional fragment thereof. In certain embodiments, the antibody or functional fragment binds to a lipoglycan-containing antigen or derivative thereof or to a bacterial antigen as described above. In certain embodiments, the antibody or functional fragment is a monoclonal antibody. In certain embodiments, the specific binding protein is a fully human monoclonal antibody or a binding fragment of a fully human monoclonal antibody. The binding fragments can include fragments such as Fab, Fab′ or F(ab′)2 and Fv. In certain embodiments, the antibody is an antibody or a functional fragment thereof can be from the same or different species.

In certain embodiments, the antibody or fragment thereof is fully human and binds to the bacterial antigen with a Kd less than 500 picomolar (pM), less than 450 pM, less than 410 pM, less than 350 pM, less than 300 pM, less than 200 pM, less than 100 pM, less than 75 pM, less than 50 pM, less than 25 pM, less than 10 pM, less than 5 pM, or less than 2 pM. Affinity and/or avidity measurements can be measured by surface plasmon resonance with the BIACORE®.

In certain embodiments, the antibody or functional fragment thereof is an IgA, IgD, IgE, IgG, or IgM. In certain embodiments, the antibody or functional fragment thereof is an IgA antibody. In certain embodiments, the antibody or functional fragment thereof is an IgA antibody produced by a dairy animal.

In certain embodiments, the method comprises mucosal immunization with the lipoglycan-containing antigen or derivative thereof or to the bacterial antigen as described above. In certain embodiments, the immune response is limited to the GI tract. In certain embodiments, the method prevents entry of the pathogenic substances into the circulation of the host.

In certain embodiments, the method comprises administering to the subject an effective amount of one or more antibodies that bind or remove the lipoglycan-containing antigen or derivative thereof, or bacterial antigen as described above, and/or administering an antibody such as, but not limited to, macrophage scavenger receptor protein (MSRP); a fragment of MSRP, wherein said fragment is capable of binding to said lipoglycan-containing antigen or lipoglycan derivative; gelsolin; a peptide comprising the amino acid sequence of the C-terminal helix of apolipoprotein CI (apoCI); daptomycin; activated charcoal; kaolinite; kaopectate; a cationic peptide; a phospholipid; a polysulphate; an endogenous binding protein or functional domain of a ficolin protein; or charcoal (e.g., activated charcoal), clay or binding resin. In certain embodiments, the antibodies that bind or remove lipoglycan-containing antigen or derivatives thereof may be administered at least 1, 2, 3, 4, 5, 6, 7, or 9 times a day.

### Pharmaceutical Compositions, Formulations and Combination Treatments

In one aspect, the present disclosure provides pharmaceutical compositions comprising an anti-lipoglycan antibody or antigen-binding fragment described herein, or a polynucleotide or the vector encoding the antibody or antigen-binding fragment, and a pharmaceutically acceptable excipient or carrier.

While it is possible to use an antibody or polynucleotide or the vector of the present invention for therapy as is, it may be preferable to administer it in a pharmaceutical formulation, e.g., in admixture with a suitable pharmaceutical excipient and/or carrier selected with regard to the intended route of administration and standard pharmaceutical practice. The excipient and/or carrier must be “acceptable” in the sense of being compatible with the other ingredients of the formulation and not deleterious to the recipient thereof. Acceptable excipients and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient and carrier can be selected with regard to the intended route of administration and standard pharmaceutical practice.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Areiams and Wilkins, Ed. K. E. Hoover). Acceptable carriers, excipients, or stabilizers are nontoxic to recipients at the dosages and concentrations used, and comprise buffers such as phosphate, citrate, and other organic acids; antioxidants including ascorbic acid and methionine; preservatives such as octadecyldimethylbenzyl ammonium chloride, hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al, Proc. Natl. Acad. Sci. USA 77:4030 (1980); and U.S. Pat. Nos. 4,485,045 and 4,544,545, Liposomes with enhanced circulation time are disclosed in U.S. Pat. No. 5,013,556.

Oral formulations readily accommodate additional mixtures, such as, e.g., milk, yogurt, and infant formula. Solid dosage forms for oral administration can also be used and can include, e.g., capsules, tablets, caplets, pills, troches, lozenges, powders, and granules. Non-limiting examples of suitable excipients include, e.g., diluents, buffering agents (e.g., sodium bicarbonate, infant formula, or other agents which allow bacteria to survive and grow [e.g., survive in the acidic environment of the stomach and to grow in the intestinal environment]), preservatives, stabilizers, binders, compaction agents, lubricants, dispersion enhancers, disintegration agents, antioxidants, flavoring agents, sweeteners, and coloring agents. Additional specific examples of suitable carriers and/or excipients include, e.g., vegetable cellulose, vegetable stearic acid, vegetable magnesium stearate, and/or silica. Those of relevant skill in the art are well able to prepare suitable solutions.

Oral delivery may also include the use of nanoparticles that can be targeted, e.g., to the GI tract of the subject, such as those described in Yun et al., Adv Drug Deliv Rev. 2013, 65(6):822-832 (e.g., mucoadhesive nanoparticles, negatively charged carboxylate- or sulfate-modified particles, etc.). Non-limiting examples of other methods of targeting delivery of compositions to the GI tract are discussed in U.S. Pat. Appl. Pub. No. 2013/0149339 and references cited therein (e.g., pH sensitive compositions [such as, e.g., enteric polymers which release their contents when the pH becomes alkaline after the enteric polymers pass through the stomach], compositions for delaying the release [e.g., compositions which use hydrogel as a shell or a material which coats the active substance with, e.g., in vivo degradable polymers, gradually hydrolyzable polymers, gradually water-soluble polymers, and/or enzyme degradable polymers], bioadhesive compositions which specifically adhere to the colonic mucosal membrane, compositions into which a protease inhibitor is incorporated, a carrier system being specifically decomposed by an enzyme present in the colon).

For oral administration, the active ingredient(s) can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

In some embodiments, the pharmaceutical composition described herein can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

In some embodiments, the pharmaceutical composition described herein can be formulated in controlled-release format, such that the active ingredient (e.g., antibody) is released primarily in the gastrointestinal tract (GI), such as in the small intestine, and preferably the ileum.

As a non-limiting example, U.S. Pat. No. 10,434,139 (incorporated by reference in its entirety for all purposes) describes a membrane-controlled dosage forms using the polymeric materials, e.g., methacrylic acid co-polymers, ammonio methacrylate co-polymers, or mixtures thereof, may be employed. Methacrylic acid co-polymers such as EUDRAGIT™ S and EUDRAGIT™ L (Evonik) are suitable for use in the controlled release formulations of the present invention. These polymers are gastroresistant and enterosoluble polymers. Their polymer films are insoluble in pure water and diluted acids. They dissolve at higher pHs, depending on their content of carboxylic acid. By using a combination of the polymers, the polymeric material can exhibit a solubility at a pH between the pHs at which EUDRAGIT™ L and EUDRAGIT™ S are separately soluble.

Other non-limiting examples of encapsulations and formulations for oral formulations of antigens include: oral vaccine formulations for ruminants described in U.S. Pat. No. 5,352,448; gelling droplets described in U.S. Pat. No. 5,674,495; aqueous solutions described in U.S. Pat. No. 5,500,161; multilamellar liposomes described in U.S. Pat. No. 6,015,576; microcapsules described in U.S. Pat. No. 5,811,128; chitosan microparticles described in Van de Lubben et al., In J. Drug Target, 2002, and Li et al., In BMC Biotechnology, 2008: polylactic acid-coglycolate (PLG) systems, described by Vajdy et al. In Immunology and Cell Biology, Vol. 82, 2004; multiple submicron oil-in-water emulsions described in U.S. Pat. No. 5,961,970; microparticulate compositions described in US Patent Application 2001/0043949; minicapsules described in PCT Application Publication WO/2008/122967 (Sigmoid Pharma Limited); and a dry powder formulation for intranasal delivery as described, for example, by Garmise et al. In AAP PharmSciTech, Vol. 7, 2006, each of the references cited above is incorporated by reference in its entirety for all purposes.

In some embodiments, the anti-lipoglycan antibodies, or the encoding nucleic acid(s), are be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are known in the art, see, e.g., Remington, The Science and Practice of Pharmacy 20th Ed. Mack Publishing (2000).

The pharmaceutical compositions described herein can be in unit dosage forms such as tablets, pills, capsules, powders, granules, solutions or suspensions, or suppositories, for oral, nasal. parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, tale, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Suitable surface-active agents (surfactant) include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent are conveniently comprise between 0.05 and 5% surface-active agent, and can be between 0.1 and 2.5%. It are be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It are be appreciated that other ingredients may be added, for example glycerol or glucose, to adjust the tonicity of the emulsion. Suitable emulsions typically contain up to 20% oil, for example, between 5 and 20%. The fat emulsion can comprise fat droplets between 0.1 and 1.0.im, particularly 0.1 and 0.5.im, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

In some embodiments, the described antibody is administered to the subject in milk or purified from milk from R. gnavus-lipoglycan immunized or R. gnavus (that produces the lipoglycan) gut colonized dairy animal, or an animal (e.g., goat or cow) genetically modified to produce these anti-lipoglycan antibodies or an animal treated with a vector that transfers the polynucleotide encoding the antibody or antigen-binding fragment for expression in the animal.

In some embodiments, the prevent disclosure provides a vaccine composition comprising a polynucleotide or the vector encoding an anti-lipoglycan antibody or antigen-binding fragment thereof. In some embodiments, the polynucleotide is an mRNA. Such vaccine compositions may be administered to the subject intranasally, inhaled, or ingested orally.

In one aspect, the invention provides a method for treating (including preventing) a disease or disorder caused by a lipoglycan-containing antigen and/or a bacterial strain associated with the lipoglycan-containing antigen in a subject in need thereof, said method comprises administering an antibody or composition (as disclosed above) in combination with administering a probiotic and/or a prebiotic composition that stimulates growth and/or activity of one or more strains of bacteria. In certain embodiments, the antibody or composition is administered before the probiotic and/or prebiotic composition(s). In certain embodiments, the antibody or composition is administered after the probiotic and/or prebiotic composition(s). In certain embodiments, the antibody or composition is administered at the same time as the probiotic and/or prebiotic composition(s). In certain embodiments, the growth is inhibited to the extent that the bacterial strains are removed from the microbiota (i.e., reduced or ablated). In certain embodiments, additional other therapeutic methods/agents (as disclosed below) can be co-administered (simultaneously or sequentially) with the combination inhibitory and stimulatory therapy to generate additive or synergistic effects.

In one embodiment of any of the above methods of the invention, the probiotic and/or prebiotic is administered to the subject by a route selected from the group consisting of oral, rectal (e.g., by enema), mucosal, sublingual, and via naso/oro-gastric gavage. In one embodiment, the probiotic is administered directly to the GI of the subject.

In some embodiments, the probiotic comprises one or more strains of bacteria from the species Faecalibacterium prausnitzii, species Bacteroides uniformis, genus Akkermansia, and/or genus Lactobacillus. In certain embodiments, the probiotic comprises at least 2, 3, 4, 5, 6, 7, 8, 9, 10, 20, 50 or more strains from the species Faecalibacterium prausnitzii, species Bacteroides uniformis, genus Akkermansia, and/or genus Lactobacillus. In some embodiments, only nonpathogenic species within the taxa qualify for use in the compositions or methods herein.

In some embodiments of any of the above methods involving administration of a probiotic composition, said probiotic composition comprises one or more OTUs which are independently characterized by, i.e., at least 95%, 96%, 97%, 98%, 99% or including 100% sequence identity to 16S rDNA or rRNA sequences of the bacteria from the species Faecalibacterium prausnitzii, species Bacteroides uniformis, genus Akkermansia, and/or genus Lactobacillus. In another embodiment, the OTUs may be characterized by one or more of the variable regions of the 16S rDNA or rRNA sequence (V1-V9). These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. (See, e.g., Brosius et al., Complete nucleotide sequence of a 16S ribosomal RNA gene from Escherichia coli, PNAS 75(10):4801-4805 (1978)). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU.

In some embodiments, the method comprises administering to the subject an effective amount of a probiotic or a prebiotic composition or a combination thereof, wherein said composition(s) stimulates growth and/or activity of one or more strains of bacteria from the species Faecalibacterium prausnitzii, species Bacteroides uniformis, genus Akkermansia, and/or genus Lactobacillus in the GI microbiota of the subject. In certain embodiments, the composition comprises at least 2, 3, 4, 5, 6, 7, 8, 9, 10, 20, 50 or more strains from the species Faecalibacterium prausnitzii, species Bacteroides uniformis, genus Akkermansia, and/or genus Lactobacillus.

In certain embodiments, the method comprises administering to the subject an effective amount of a probiotic or a prebiotic composition or a combination thereof, wherein said composition(s) stimulates growth and/or activity or includes one or more strains of bacteria from one or more phyla selected from the group consisting of Firmicutes, Bacteroidetes, and/or Verrucomicrobia in the GI microbiota of the subject. In certain embodiments, the composition comprises at least 2, 3, 4, 5, 6, 7, 8, 9, 10, 20, 50 or more strains from these taxa.

In certain embodiments, the method comprises administering to the subject an effective amount of a probiotic or a prebiotic composition or a combination thereof, wherein said composition(s) stimulates growth and/or activity or includes of one or more strains of bacteria from one or more classes selected from the group consisting of Clostridia, Bacteroidetes, Verrucomicrobiae, and/or Bacilli in the GI microbiota of the subject. In certain embodiments, the composition comprises at least 2, 3, 4, 5, 6, 7, 8, 9, 10, 20, 50 or more strains from these taxa.

In certain embodiments, the method comprises administered to the subject an effective amount of a probiotic or a prebiotic composition or a combination thereof, wherein said composition(s) stimulates growth and/or activity or includes one or more strains of bacteria from one or more orders selected from the group consisting of Clostridiales, Bacteroidales, Verrucomicrobiales and/or Lactobacillales in the GI microbiota of the subject. In certain embodiments, the composition comprises at least 2, 3, 4, 5, 6, 7, 8, 9, 10, 20, 50 or more strains from these taxa.

In certain embodiments, the method comprises administered to the subject an effective amount of a probiotic or a prebiotic composition or a combination thereof, wherein said composition(s) stimulates growth and/or activity or includes one or more strains of bacteria from one or more families selected from the group consisting of Clostridiaceae, Bacteroidaceae, Verrucomicrobiaceae, and/or Lactobacillaceae in the GI microbiota of the subject. In certain embodiments, the composition comprises at least 2, 3, 4, 5, 6, 7, 8, 9, 10, 20, 50 or more strains from these taxa.

In certain embodiments, the method comprises administered to the subject an effective amount of a probiotic or a prebiotic composition or a combination thereof, wherein said composition(s) stimulates growth and/or activity or includes of one or more strains of bacteria from one or more genera selected from the group consisting of Faecalibacterium, Bacteroides, Akkermansia, and Lactobacillus in the GI microbiota of the subject. In certain embodiments, the composition comprises at least 2, 3, 4, 5, 6, 7, 8, 9, 10, 20, 50 or more strains from these taxa.

In certain embodiments, the method comprises administered to the subject an effective amount of a probiotic or a prebiotic composition or a combination thereof, wherein said composition(s) stimulates growth and/or activity or includes one or more strains of bacteria from one or more species selected from the group consisting of Faecalibacterium prausnitzii and/or Bacteroides uniformis in the GI microbiota of the subject. In certain embodiments, the composition comprises at least 2, 3, 4, 5, 6, 7, 8, 9, 10, 20, 50 or more strains from these taxa.

Within a given composition, different bacterial strains can be contained in equal amounts (even combination) or in various proportions (uneven combinations) needed for achieving the maximal biological activity. For example, in a bacterial composition with two bacterial strains, the strains may be present in from a 1:10,000 ratio to a 1:1 ratio, from a 1:10,000 ratio to a 1:1,000 ratio, from a 1:1,000 ratio to a 1:100 ratio, from a 1:100 ratio to a 1:50 ratio, from a 1:50 ratio to a 1:20 ratio, from a 1:20 ratio to a 1:10 ratio, from a 1:10 ratio to a 1:1 ratio. For bacterial compositions comprising at least three bacterial strains, the ratio of strains may be chosen pairwise from ratios for bacterial compositions with two strains. For example, in a bacterial composition comprising bacterial strains A, B, and C, at least one of the ratios between strain A and B, the ratio between strain B and C, and the ratio between strain A and C may be chosen, independently, from the pairwise combinations above. In one specific embodiment, the invention encompasses administering two or more bacteria-containing compositions to the same subject. Such compositions can be administered simultaneously or sequentially.

In one embodiment of any of the above methods of the invention, the probiotic is administered in a therapeutically effective amount. The dosages of the microbiota inoculum and/or probiotic composition administered in the methods of the invention will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, or more than once a day, semi-weekly, etc., to maintain an effective dosage level. It is contemplated that a variety of doses will be effective to achieve colonization, e.g. 10⁶, 10⁷, 10⁸, 10⁹, and 10¹⁰ CFU for example, can be administered in a single dose. Lower doses can also be effective, e.g., 10⁴, and 10⁵ CFU.

The probiotic composition useful in any of the above methods can comprise, without limitation, e.g., live bacterial cells, conditionally lethal bacterial cells, inactivated bacterial cells, killed bacterial cells, spores (e.g., germination-competent spores), recombinant carrier strains, cell extract, and bacterially-derived products (natural or synthetic bacterially-derived products such as, e.g., bacterial antigens or bacterial metabolic products).

Bacterial strains administered in probiotic compositions according to the methods of the present invention can comprise live bacteria. One or several different bacterial inoculants can be administered simultaneously or sequentially (including administering at different times). Such bacteria can be isolated from the GI tract and grown in culture. The present invention also comprises administering “bacterial analogues”, such as recombinant carrier strains expressing one or more heterologous genes derived from the relevant bacterial species. The use of such recombinant bacteria may allow the use of lower therapeutic amounts due to higher protein expression. Non-limiting examples of recombinant carrier strains useful in the methods of the present invention include E. coli and Lactobacillus, Bacteroides and Oxalobacter. Methods describing the use of bacteria for heterologous protein delivery are described, e.g., in U.S. Pat. No. 6,803,231.

In certain embodiments, the probiotic comprises a preparation of the GI microbiota of a healthy subject. A suitable donor might have no known infections or colonizations by disease associated microbes and viruses. A spouse or family method without evidence of disease might be suitable. It might be best to transfer a carefully selected collection or consortium of commensal bacteria, with or without pretreatment that would facilitate colonization and prevent recurrence of the disease-associated taxa (i.e., species and strain).

Methods for producing bacterial compositions of the invention may include three main processing steps, combined with one or more mixing steps. The steps are: organism banking, organism production, and preservation. For banking, the strains included in the bacterial compositions of the invention may be (1) isolated directly from a specimen or taken from a banked stock, (2) optionally cultured on a nutrient agar or broth that supports growth to generate viable biomass, and (3) the biomass optionally preserved in multiple aliquots in long-term storage. The bacterial suspension can be freeze-dried to a powder and titrated. After drying, the powder may be blended to an appropriate potency, and mixed with other cultures and/or a filler such as microcrystalline cellulose for consistency and ease of handling, and the bacterial composition formulated as provided herein.

In one embodiment of any of the above methods of the invention, the probiotic is delivered to the subject in a form of a suspension, a pill, a tablet, a capsule, or a suppository. In another embodiment, the probiotic is delivered to the subject in a form of a liquid, foam, cream, spray, powder, or gel. In yet another embodiment, the probiotic is delivered to the subject in a saline suspension for use in feeding tubes, transmission via nasogastric tube, or enema. If live bacteria are used, the carrier should preferably contain an ingredient that promotes viability of the bacteria during storage.

The formulation can include added ingredients to improve palatability, improve shelf-life, impart nutritional benefits, and the like. If a reproducible and measured dose is desired, the bacteria can be administered by a rumen cannula.

In one embodiment of any of the above methods of the invention, the bacterial inoculum is delivered to the subject in a form of a composition which comprises (i) a carrier and/or excipient and/or (ii) one or more prebiotic agents which stimulate growth and/or activity of one or more bacteria present in the composition. In one specific embodiment, said composition comprises an excipient or a carrier that optimizes the seeding of the transferred microbiota.

In one embodiment of any of the above methods involving administration of a probiotic composition, said probiotic composition is reconstituted from a lyophilized preparation. In one embodiment of any of the above methods involving administration of a probiotic composition, said probiotic composition comprises a buffering agent to adjust pH.

In one embodiment, the probiotic composition comprises a buffering agent (e.g., sodium bicarbonate, infant formula, or other agents which allow bacteria to survive and grow [e.g., survive in the acidic environment of the stomach and to grow in the intestinal environment]), along with preservatives, stabilizers, binders, compaction agents, lubricants, dispersion enhancers, disintegration agents, antioxidants, flavoring agents, sweeteners, and coloring agents.

In one embodiment of any of the above methods involving administration of a probiotic composition, the probiotic composition is administered conjointly with a prebiotic which stimulates growth and/or activity of bacteria contained in the probiotic composition (conjoint biotic administration). Non-limiting examples of useful prebiotics include, e.g., galactose, β-N-Acetyl-α-glucosamine, pyroglutamtamic acid, arginine, serine, glycine, fructooligosaccharides (FOS), galactooligosaccharides (GOS), human milk oligosaccharides (HMO), Lacto-N-neotetraose, D-Tagatose, xylo-oligosaccharides (XOS), arabinoxylan-oligosaccharides (AXOS), N-acetylglucosamine, N-acetylgalactosamine, glucose, arabinose, maltose, lactose, sucrose, cellobiose, amino acids, alcohols, resistant starch (RS), electrolytes and any combinations thereof. In some embodiments, the electrolytes can modulate or balance the pH. In one specific embodiment, the probiotic and prebiotic are administered in one composition, or simultaneously as two separate compositions, or sequentially.

In one aspect, the invention provides a method for treating (including preventing) a disease or disorder caused by a lipoglycan-containing antigen and/or a bacterial strain associated with the lipoglycan-containing antigen in a subject in need thereof, said method comprises administering an antibody or composition (as disclosed above) in combination with administering a bacteriophage against one or more strains of bacteria associated with the lipoglycan-containing antigen. Phage therapy can minimize the population of the strain that caused an overgrowth, whilst allowing other strains to increase their population, which were previously lowered by the overgrowth. Sequence analysis of R. gnavus strains based on the inventors' work identified sequences likely to be prophage, suggesting there are naturally occurring bacteriophage for this species. Other research has also shown that members of the bacterial family Lachnospiraceae have phage, and R. gnavus is of this family. Thus, natural lytic phages against one or more pathogenic R. gnavus stains may be used in combination with the antibody or composition described herein to treat the one or more target diseases or disorders.

It is also contemplated that when used to treat a disease or disorder caused by a lipoglycan-containing antigen and/or a bacterial strain associated with the lipoglycan-containing antigen, the compositions and methods of the present invention can be utilized with other therapeutic methods/agents suitable for the same or similar diseases or disorders. Such other therapeutic methods/agents can be co-administered (simultaneously or sequentially) to generate additive or synergistic effects. Suitable therapeutically effective dosages for each agent may be lowered due to the additive action or synergy.

In one embodiment of any of the above methods of the invention, the method further comprises administering to the subject one or more additional compounds selected from the group consisting of immuno-suppressives, biologicals, probiotics, prebiotics, and cytokines (e.g., IFN or IL-22).

In certain embodiments, the compositions can be administered with an effective amount of anti-inflammatory drugs (NSAIDs), antimalarial agents, corticosteroids, azathioprine, mycophenolate, methotrexate, leflunomide, belimumab, and Vitamin D.

In certain embodiments, the antimalarial agent can be used to treat an autoimmune disease. In certain embodiments, the antimalarial drug is amodiaquine, chloroquine, chlorproguanil HCl, halofantrine HCl, mefloquine HCl, proguanil HCl, pyrimethamine, quinine sulfate, or hydroxychloroquine. In certain embodiments, the antimalarial agent is hydroxychloroquine.

In certain embodiments, the corticosteroid is prednisone, hydrocortisone, prednisolone, dexamethasone, alclometasone dipropionate, amcinonide, beclamethasone dipropionate, betamethiasone benzoate, betamethasone dipropionate, betamethasone valerate, budesonide, clobetasol propionate, clobetasone butyrate, desonide, desoxymethasone, diflorasone diacetate, diflucortolone valerate, flumethasone pivalate, fluclorolone acetonide, fluocinolone acetonide, fluocinonide, fluocortin butyl, fluocortolone preparations, fluprednidene acetate, flurandrenolone, halcinonide, hydrocortisone, hydrocortisone acetate, hydrocortisone butyrate, methylprednisolone acetate, mometasone furoate, or triamcinolone acetonide. In certain embodiments, the corticosteroid is prednisone or hydrocortisone or prednisolone or dexamethasone.

As a non-limiting example, the invention can be combined with other therapies that block inflammation (e.g., via binding or blockade of IL1, INFα/β, IL6, TNF, IL23, etc.) or inhibitors of specific cytoplasmic tyrosine kinases alone or in combination with a compound that is a Janus kinase inhibitor.

The methods and compositions of the invention can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.). The methods of the invention can be also combined with other treatments that possess the ability to modulate NKT cell function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e).

In certain embodiments, a conditional lethal bacterial strain can be utilized as the inoculant or to deliver a recombinant construct. Such a conditional lethal bacterial strain survives for a limited time typically when provided certain nutritional supplements. It is contemplated that such a supplement could be a liquid, formulated to contain the nutritional component necessary to keep the bacteria alive. It is further contemplated that a patient/subject would drink such a supplement in intervals to keep the bacteria alive. Once the supplement is depleted, the conditional lethal bacteria die. Methods relating to conditional lethal strains of H. pylori are described in U.S. Pat. No. 6,570,004. In certain embodiments, the methods entail use of a bacteriophage that modulates the representation or the specific gene product expression of the bacterial strain (e.g., the strain of R. gnavus).

Spores used in the compositions of the invention can be isolated, for example, by solvent treatments (e.g., using partially miscible, fully miscible or an immiscible solvent), chromatographic treatments (e.g., using hydrophobic interaction chromatography (HIC) or an affinity chromatography), mechanical treatments (e.g., blending, mixing, shaking, vortexing, impact pulverization, and sonication), filtration treatments, thermal treatments (e.g., 30 seconds in a 100° C. environment followed by 10 minutes in a 50° C.), irradiation treatments (e.g., with ionizing radiation, typically gamma irradiation, ultraviolet irradiation or electron beam irradiation provided at an energy level sufficient to kill pathogenic materials while not substantially damaging the desired spore populations), centrifugation and density separation treatments (e.g., using density or mobility gradients or cushions (e.g., step cushions), such as, e.g., CsCl, Percoll, Ficoll, Nycodenz, Histodenz or sucrose gradients). It is generally desirable to retain the spore populations under non-germinating and non-growth promoting conditions and media, in order to minimize the growth of pathogenic bacteria present in the spore populations and to minimize the germination of spores into vegetative bacterial cells.

### EXAMPLES

The following examples are provided to further describe some of the embodiments disclosed herein. The examples are intended to illustrate, not to limit, the disclosed embodiments.

### Example 1. RG Strains are Subgrouped Based on Expression Versus Non-Expression of a Conserved Lipoglycan

Gut microbiomes contain complex interdependent communities that in health provide layered tiers for nutritional and immune regulatory benefits. Imbalances (or dysbiosis) have been implicated in a growing number of clinical conditions but only in a handful of cases have expansions of specific bacteria, defined by species or genus, been implicated, and in even fewer cases have actual pathogenic pathways been identified. In studies of a clinically diverse cohort of female patients with Systemic Lupus Erythematosus (SLE), inventors of the present disclosure discovered the first correlation between overrepresentation of an obligate anaerobe, Blautia (Ruminococcus) gnavus (RG) with SLE disease activity (Azzouz et al., 2019). More intriguingly, even amongst Lupus patients these RG expansions were more common in patients with Lupus nephritis (LN), which affects more than half of patients and is associated with amongst the greatest morbidity and mortality (Azzouz et al., 2019). Notably, the association of RG with LN was also later independently documented in a large cohort of untreated Chinese Lupus patients (Chen et al., 2020). In more recent longitudinal studies of the cohort, Lupus patients were found to have inherently unstable gut microbiota communities and almost half of clinical flares of renal disease were temporally associated with ephemeral RG blooms in SLE patients.

One of the estimated 53 most common human intestinal colonizers, RG are early colonizers that are detectable in most infants by 24 months of age (Sagheddu et al., 2016). In adults, RG is present in at least 90% of individuals from North American and Europe, although generally at stable low levels at or below 0.1% abundance (Qin et al., 2010; Schloissnig et al., 2013).

Based on genomic phylogenetic analysis, RG has been reassigned to the Phylum Firmicutes, family Lachnospiraceae and genus Blautia of spore-forming obligate anaerobes. As a species, RG is quite distinct from other taxa at both the genome as well as the 16S rDNA gene sequence level (Sorbara et al., 2020). In healthy adults, RG is a keystone species present in 90% or more of adults (Qin et al., 2010; Schloissnig et al., 2013), playing pleotropic roles in host metabolism and immunity (reviewed in (Vacca et al., 2020)), including the conversion of primary to secondary bile acids (Buffie et al., 2015), and production of the short-chain fatty acids (SCFAs) that aid immune regulation (Arpaia et al., 2013), and hence most RG strains are considered pro-homeostatic.

In general, most reports of disease-associated RG abundance variations in microbiota communities have been limited to correlative studies, with limited exception (Henke et al., 2021). The in vivo effects of RG isolates on host immunity have largely focused on the Human Microbiome Project designated type-specific RG strain, VPI C7-9, also termed ATCC29149 (referred to in the present studies as RG1) (Hoffmann et al., 2015; Bell et al., 2019; Yang, 2020) isolated from stool of a healthy donor (Moore, 1976). While increased intestinal abundance of the RG species alone might explain associations with Lupus disease flares (Azzouz et al., 2019), it was hypothesized herein that there may be important differences in the pathogenetic potential of strains from healthy individuals from those colonizing LN patients. Moreover, inventors of the present disclosure have discovered that active Lupus Nephritis patients have gut RG expansions, with concurrent high serum IgG antibodies to a novel lipoglycan produced by RG strains residing within the host (termed S107-48 and S47-18), which provide circumstantial evidence of involvement in autoimmune pathogenesis.

A key goal of the present disclosure was to compare the effects of different genome-defined RG strains on the host following in vivo intestinal colonization, with an emphasis on whether gut-barrier function was affected. Whereas all RG strains evaluated had the capacity to colonize the mouse gut, there were dramatic differences in the effects of individual strains on intestinal permeability, which was found to mediated by a zonulin-dependent mechanism. Indeed, strains isolated from clinically active Lupus patients reproducibly induced these changes. Moreover, neonatal murine colonization with Lupus RG strains resulted in microbial translocation and systemic antibody responses to RG-specific antigens and induction of Lupus autoantibodies. These findings provide a mechanistic rationale for the previously reported linkage between RG intestinal expansions and Lupus pathogenesis (Azzouz et al., 2019).

Studies of the present disclosure investigated differences in the in vivo pathogenicity properties of Blautia (Ruminococcus) gnavus (RG) strains isolated from a fecal sample of a healthy donor, VPI C7-9 (here termed RG1) and from a colonic biopsy, CC55_001C (here termed RG2), with comparisons to the RG strains, S107-48 and S47-18 obtained from two SLE patients with RG blooms at the time of flare of renal disease.

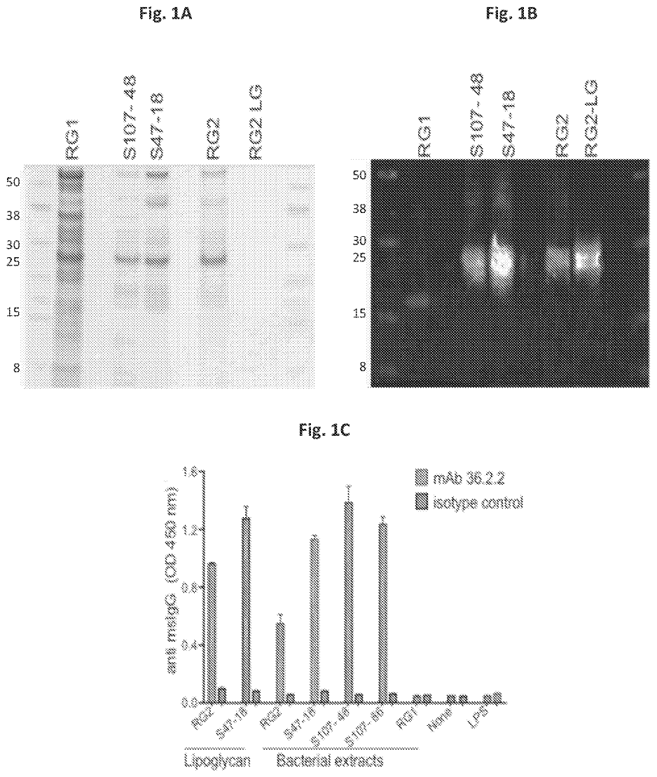

To provide an independent approach to investigate the antigenic diversity expressed by different RG strains, murine monoclonal antibodies were generated by RG bacterial immunization in accordance with below-described methods. By enzyme-linked immunosorbent assay (ELISA), the resultant mAb 36.2.2 was strongly reactive with the purified LGs from the S47-18 strain used for the immunization boost and the previously reported purified LG from the RG2 strain (FIG. 1). In addition, this mAb was reactive with extracts of the RG2 strain and Lupus strains, S47-18, S107-48 and S107-86, that were isolated from two different LN patients (FIG. 1). This antibody, however, was non-reactive with the RG1 type strain. RG strains were therefore assignable to two subgroups based on the presence or absence of a cell wall associated lipoglycan with the same oligoband MW distribution and shared antigenic determinants that are recognized by the mAb 36.2.2.

### Example 2. RG Persistence in Germ-Free Mice with Transmission to Litters

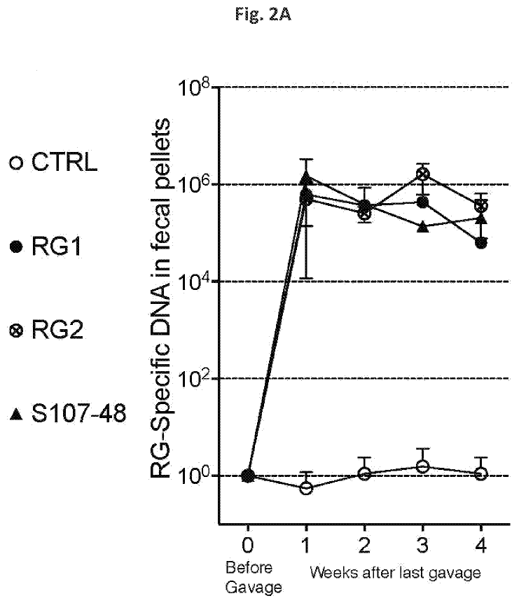

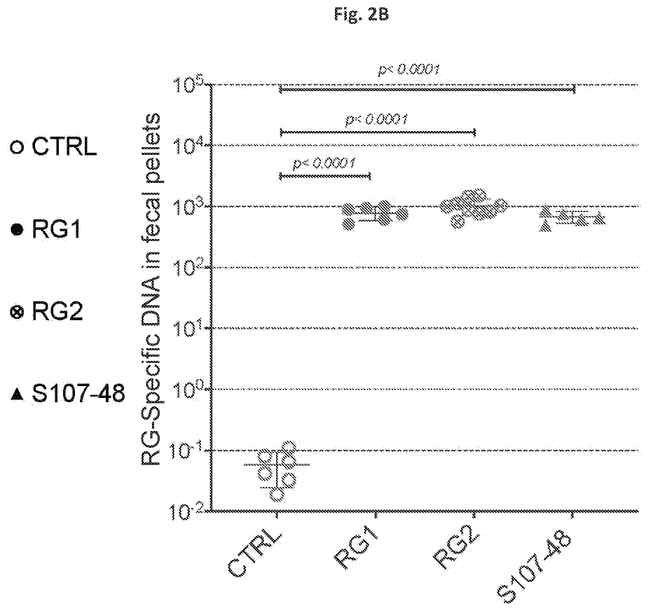

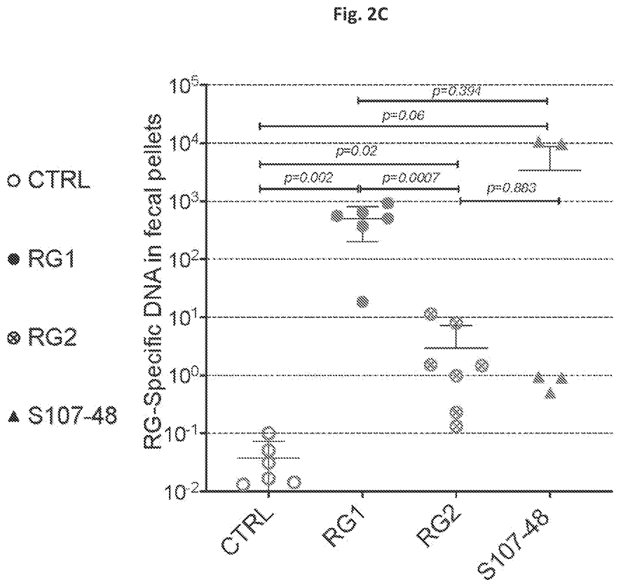

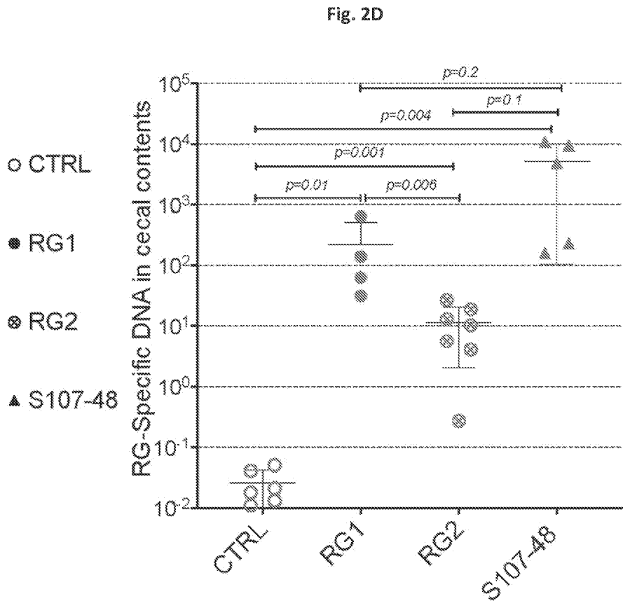

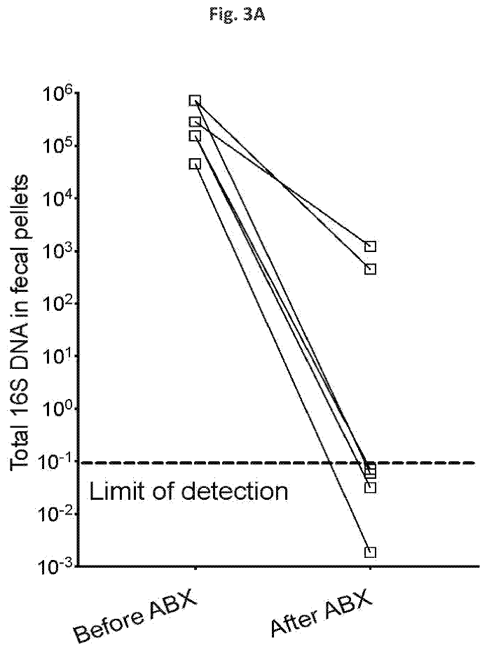

To initiate these studies, in a gnotobiotic system, a standard gavage protocol was used to colonize groups of germ-free C57BL/6 mice with different RG strains. Due in part to the practical challenges of accurate colony-forming unit (CFU) determinations from this obligate anaerobe, a previously described RG rDNA species genomic-specific qPCR assay, which was validated to be highly RG species-specific (Png et al., 2010), was adapted for use in the present Example. Whereas RG was undetectable in the fecal pellets of naïve germ-free C57BL/6 mice, following gavage with different RG strains, high levels were detectable in sequential samples obtained over the four weeks following gavage (FIG. 2A). There initially were no differences in levels of fecal RG representation with the different strains, nor were there differences based on the sex of the recipient mice (e.g., FIG. 1A and FIG. 2A).

Earlier studies in mice have demonstrated that adult germ-free mice have immune defects, whereas colonization of neonatal mice results in exposure to antigens of colonizing bacterial species that can affect both the host innate immunity, as well as lymphocyte development (Wesemann, 2015). Breeding pairs of these formerly germ-free (GF) mice that had each been colonized with individual RG strains were therefore mated. Each of these strain-colonized pairs yielded litters, which were then weaned, and then individual fecal samples were collected and tested. The results revealed roughly equivalent high levels of RG-species specific 16S rDNA genes (FIG. 2B).

To investigate the potential persistence of RG colonization, fecal pellets were also obtained at about 3-months of age, and RG levels were found to diverge based on the colonizing RG strains. Compared to controls, the highest mean RG levels were found in mice colonized with the RG1 strain (p=0.002), while lower but still significant levels were detected in mice colonized with the RG2 strain (p=0.02) (FIG. 2C). Although each was above the level found in the non-colonized control mice, the RG levels detected in S107-48 Lupus strain colonized mice differed greatly (FIG. 2C) and were quantitatively different compared to controls (p=0.06). Interestingly, in this group fecal RG levels were more heterogeneous, and higher in two of the three females and lower in the two S107-48 colonized male mice (FIG. 2C).

In health, species of the Lachnospiraceae family are known to differentially colonize regional sites within the intestine (Donaldson et al., 2016). Therefore, after sacrifice, the cecal luminal contents were sampled and significant levels of RG-specific genomic DNA were documented in mice colonized with each of the three RG strains (FIG. 2D). Notably, the highest mean levels were documented for the Lupus S107-48 strain, followed by the RG1 and then the RG2 strain that had been isolated from healthy individuals. While it was not entirely unexpected that human RG isolates might not persist indefinitely in the murine intestine, it was intriguing to find mice with much higher levels due to cecal persistence at a time of waning RG representation in fecal pellets in many of these same individual mice.

### Example 3. RG Bacteria Efficiently Colonizes Antibiotic-Preconditioned SPF Mice

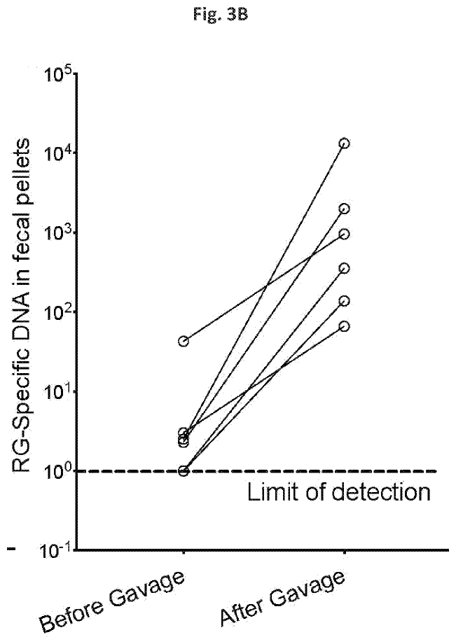

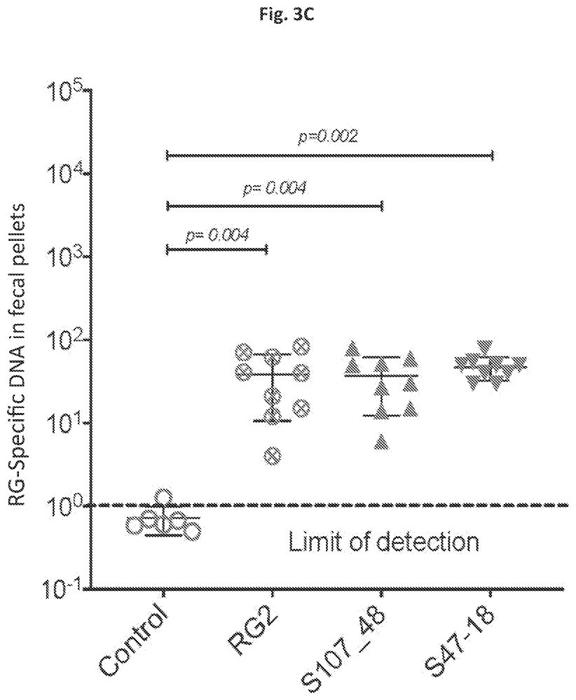

The present Example aimed to investigate the capacity of RG strains to colonize immunocompetent adult mice that were bred and raised under SPF conditions. To reduce the intestinal bacterial burden, a previously validated combination oral antibiotic regimen was employed. Bacterial depletion was then monitored using a total 16S rDNA qPCR assay known to broadly amplify bacterial taxa of diverse phylogenetic origins (see methods) (FIG. 3A). By this approach, findings showed that community abundance was reduced at least 100-fold in the antibiotic-treated mice after a one-month duration of antibiotic treatment (FIG. 3A). Individual mating pairs were then gavaged with different RG strain cultures. In comparisons of paired fecal pellet samples obtained before and two weeks after gavage, although there was inter-individual variation, the RG-specific qPCR assay demonstrated significant levels of RG colonization in each of the recipient mice (FIG. 3B). After mating of pairs colonized with the same RG strain, fecal pellets of 5-week-old litters contained an abundance of RG genomic DNA in the RG1, RG2, S107-48 and S47-18 strain monocolonized SPF groups that was roughly equivalent across the groups, and significantly greater than in uncolonized control mice where RG was undetectable. These studies showed the capacity of diverse RG strains to colonize mice raised under SPF conditions.

### Example 4. Lupus RG Strains Induce Increased Gut Permeability

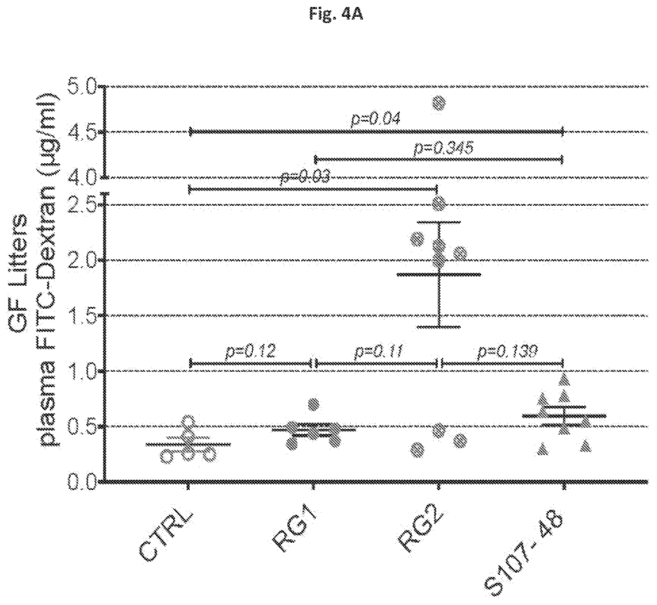

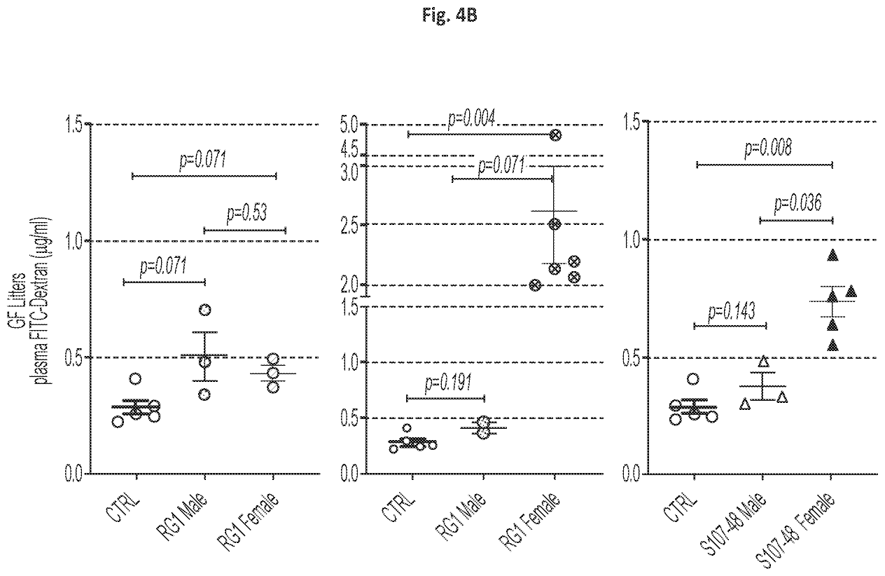

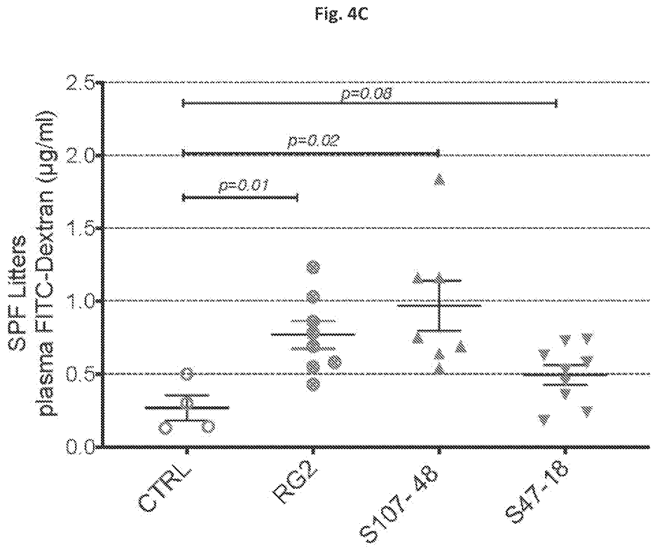

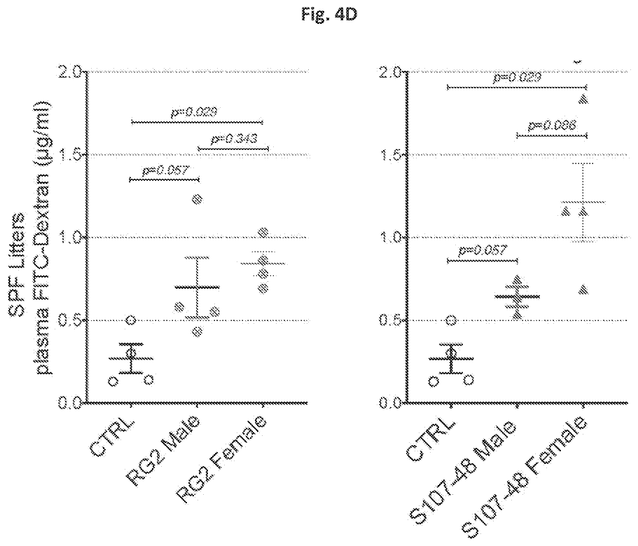

To investigate the potential influences of intestinal colonization on gut barrier integrity, individual mice were gavaged with a standardized dose of FITC-labeled dextran of 4000 Dalton (Da) molecular weight (FD4), then blood samples were subsequently obtained to assess altered intestinal permeability (FIG. 4). In the litters from GF mice colonized with RG, little or no leakage was detected in the weaned pups colonized by RG1. In contrast, there was evidence of increased permeability in both the litters colonized with the RG2 strain and the Lupus strain, S107-48 (FIG. 4A). There were no differences in plasma FD4 levels in male versus female mice colonized with the RG1 strain (FIG. 4B). Strikingly, the female mice colonized with the RG2 strain and those with the Lupus S107-48 strain had substantially higher levels of post-challenge plasma FD4, consistent with greater intestinal permeability in these RG strains (FIG. 4B).

Mating of the pairs colonized with the different strains resulted in litters, except for the RG1-colonized mice that did not yield progeny. In the litters of RG-colonized SPF mice, evidence of increased gut permeability was identified in the mice colonized by the RG2 and the Lupus S107-48 RG strains (FIG. 4C). There was a numerical trend toward greater intestinal permeability in the female progeny compared to the male progeny colonized by the RG2 strain. Compared to controls, a numerical trend towards higher mean levels was also documented in male mice colonized by the Lupus S107-48 RG strain (p=0.057). The highest statistically significant mean levels of intestinal permeability were demonstrated in the female mice colonized with the Lupus S107-48 RG strain (p=0.029) (FIG. 4D). These data demonstrated there is a female bias for induction of impaired gut barrier function resulting from colonization by the S107-48 RG Lupus strain.

### Example 5. RG Translocation to MLN in RG-Colonized Mice with Altered Intestinal Permeability

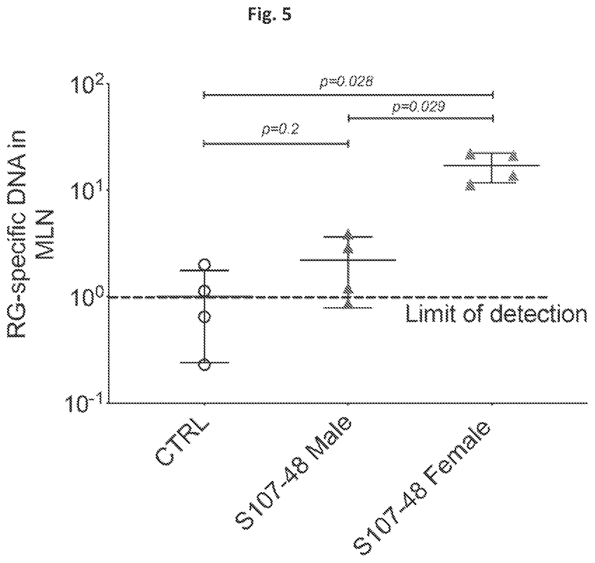

Based on above-described evidence of impaired gut barrier, tissue extracts of colonized mice were examined using the RG-specific 16S assay. Reiterating the patterns seen in the intestinal permeability assays (FIG. 4), significant translocation of RG-specific genomic DNA was found in the female mice (p=0.001), with RG DNA levels also significantly above the levels found in male mice littermates colonized with the same S107-48 Lupus strain (p=0.002) (FIG. 5). However, persistent RG DNA was not found in splenic extracts from the same mice. These findings suggest that bacterial fragments can traverse the gut barrier because of colonization with some, but not all RG strains, and that female mice are more susceptible to RG-mediated breaches in the gut barrier.

### Example 6. Systemic Immune and Autoimmune Consequences of RG-Mediated Breaches of the Gut Barrier

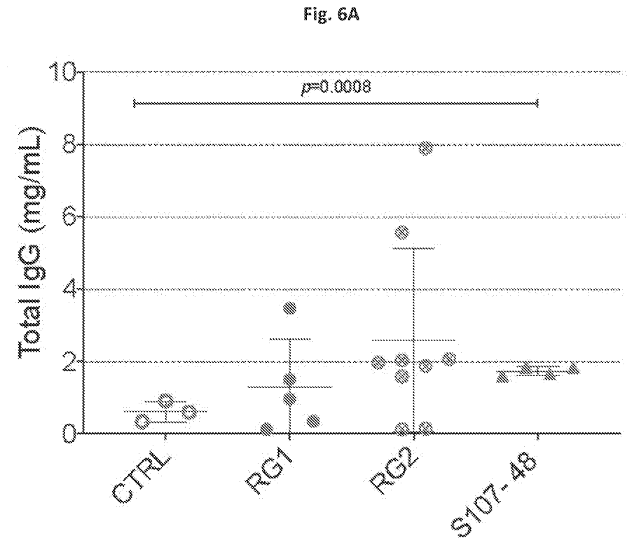

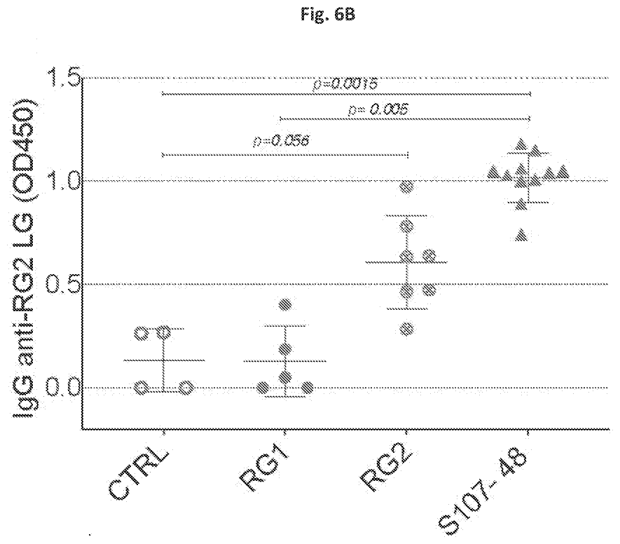

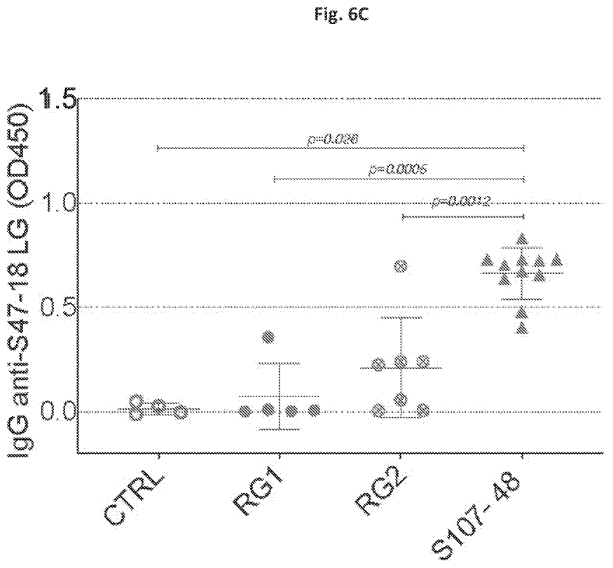

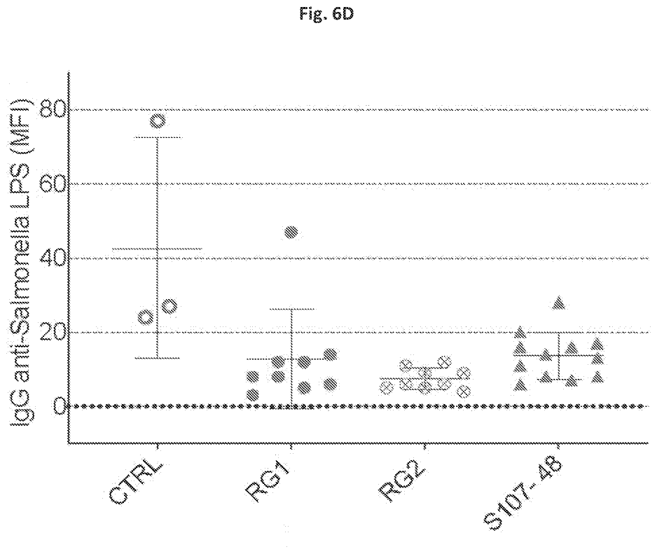

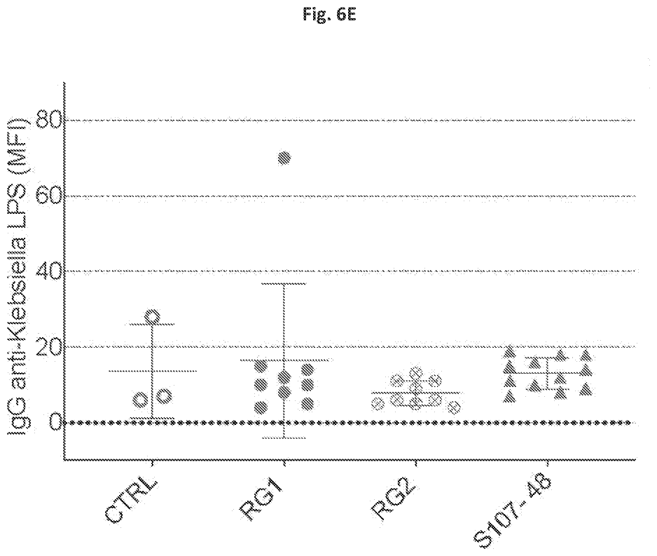

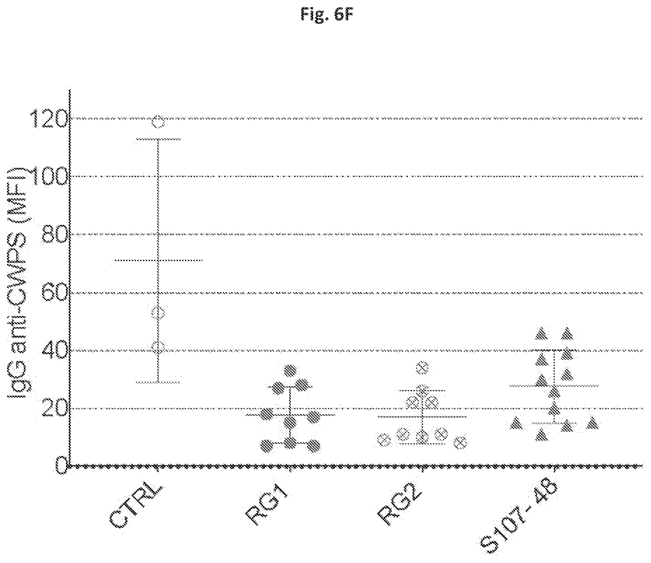

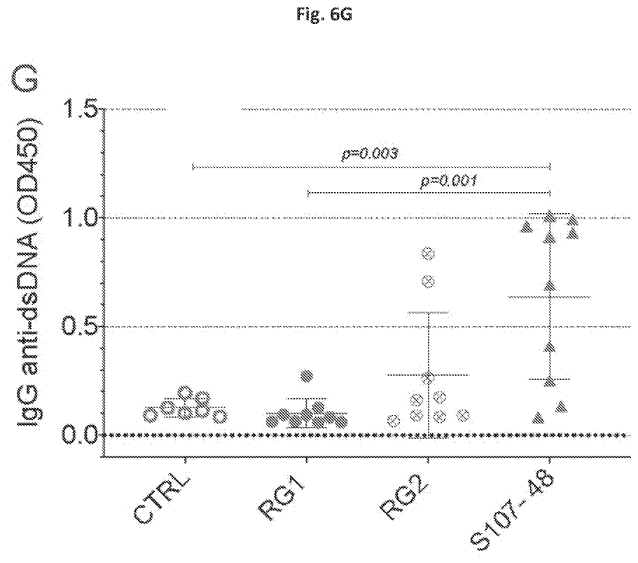

Based on evidence of an induced breach in the functional gut barrier, mice from litters of RG colonized previously germ-free mice were evaluated for specific systemic immunorecognition of RG-associated antigens. In these studies, RG colonization appeared to raise total IgG levels compared to noncolonized controls (FIG. 6A), with significantly raised levels of serum IgG antibodies in S107-48 colonized mice that recognized the purified RG2 strain lipoglycan and the S47-18 strain lipoglycan antigens (FIG. 6B and FIG. 6C). In part, these findings are consistent with antigenic cross-reactivity between lipoglycans isolated from the RG2 and Lupus-associated RG strains, while extracts of the RG1 strain were non-cross-reactive (Azzouz et al., 2019). Furthermore, there was no detectable reactivity with LPS isolated from Klebsiella or Salmonella (FIG. 6D and FIG. 6E), nor with pneumococcal cell wall C-polysaccharide (FIG. 6F), which suggests that the anti-RG reactivity was antigen-specific.

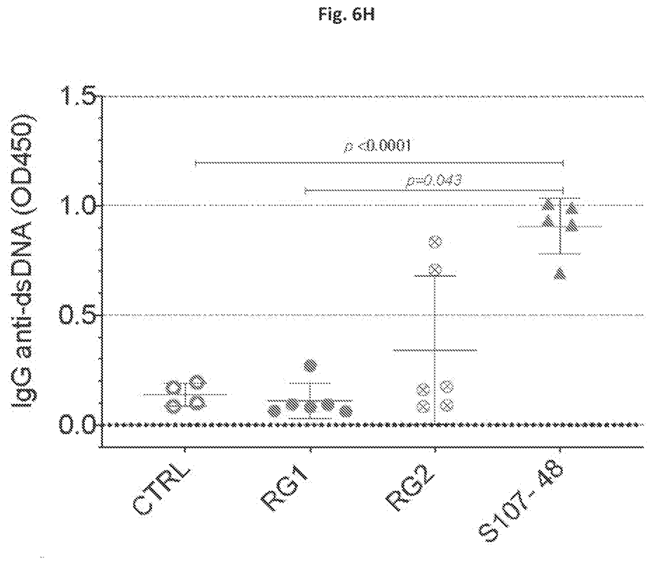

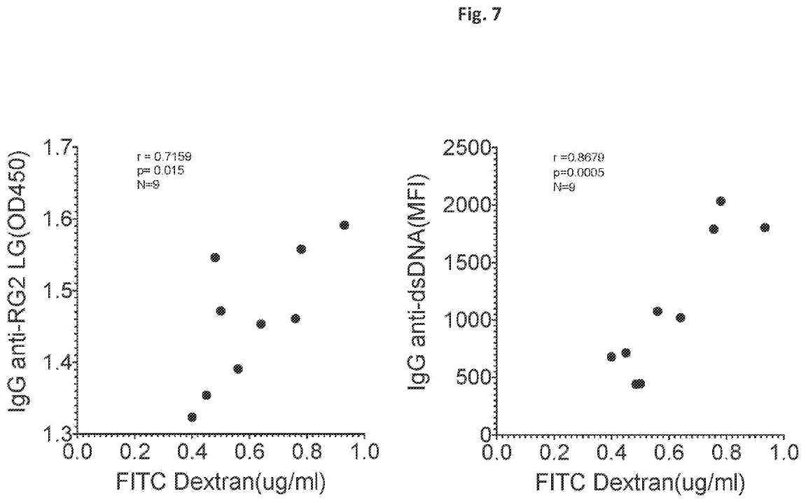

Based on the reported associations in Lupus patients between circulating levels of circulating IgG anti-RG lipoglycan and Lupus anti-nuclear antibody production (ANA), plasma samples were assayed for binding reactivity with native thymic genomic DNA (dsDNA). These studies demonstrated significantly raised levels of IgG anti-DNA antibodies in mice after neonatal colonization with S107-48 Lupus strain (p=0.003) (FIG. 6G), which was primarily due to the raised anti-DNA antibody levels in the female mice (p<0.0001) (FIG. 6H). Furthermore, in individual S107-48 colonized mice the level of intestinal permeability directly correlated with IgG anti-RG2 lipoglycan antibodies (Spearman r=0.7159, p=0.015), and with the level of IgG anti-native DNA autoantibodies (r=0.8679, p=0.0005) (FIG. 7). These studies therefore demonstrate that female mice are more susceptible to Lupus RG-induced impairment in the gut barrier, with immunologic consequences including induction of serum antibodies to RG strain-associated lipoglycan determinants, and Lupus anti-DNA autoantibody production.

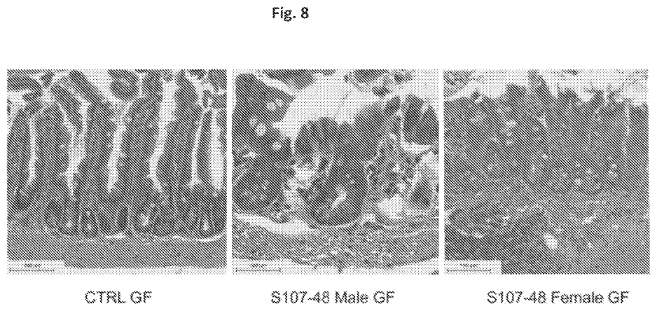

To further investigate the mechanisms responsible for increased gut permeability, mice were sacrificed at 14 weeks of age, and sections of the small intestine were harvested for subsequent histopathologic examination. These studies demonstrated that unlike the normal morphology of intestinal villi and crypts seen in the control mice raised under GF conditions, neonatal colonization with the Lupus S107-48 RG strain in littermates of both sexes was associated with histologic abnormalities such as areas of shortened epithelial villi and changes in the submucosa (FIG. 8A). These findings are consistent with the above-described evidence that colonization with some RG strains (i.e., strains that are lipoglycan producing) induces functional intestinal barrier impairment.

### Example 7. RG Strain-Specific Increases in Gut Permeability is Reversible with Oral Treatment with Larazotide

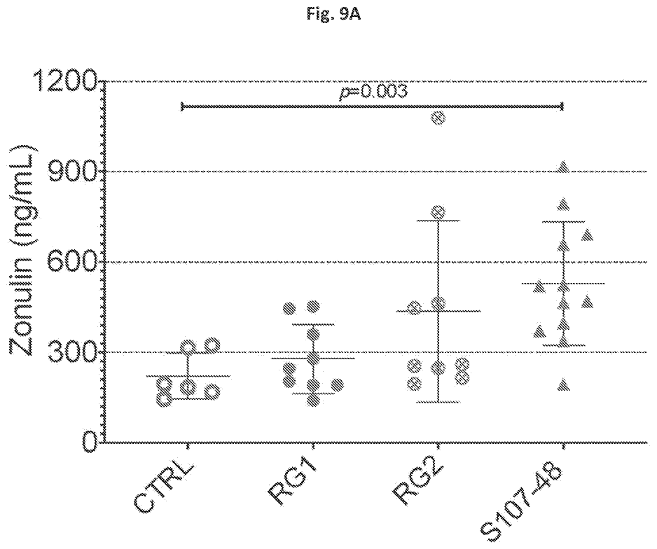

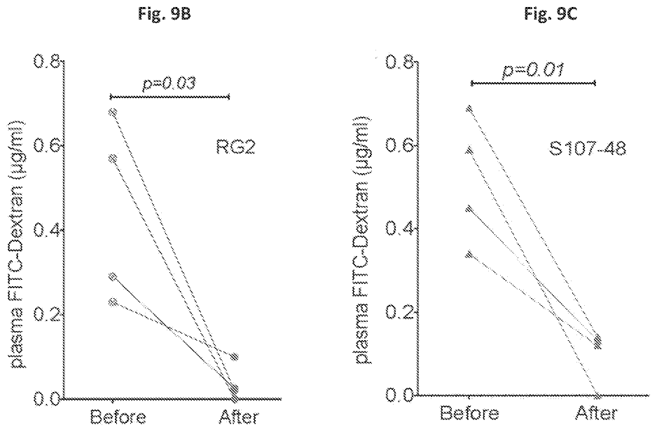

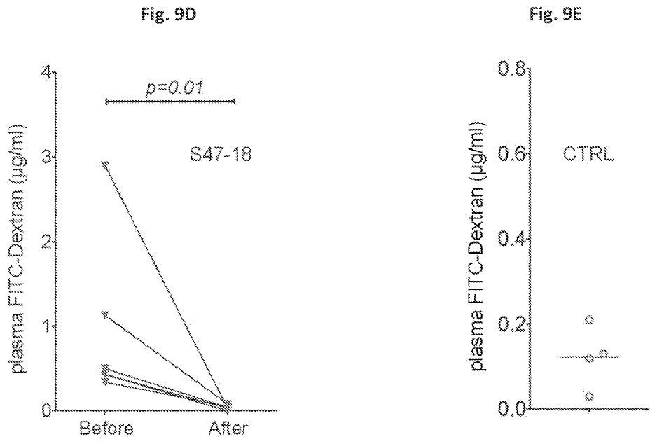

To evaluate a potential causal role of elevated serum zonulin levels in the functional abnormalities documented in S107-48 RG strain colonized mice, the FD4 challenge assay was repeated, and then groups of mice were treated for ten days with oral administration of larazotide acetate (AT-1001), an octapeptide, originally identified from studies of the zonula occluden toxin by Vibrio cholera (Sturgeon and Fasano, 2016) (FIG. 8A and FIG. 8B). Larazotide treatment resulted in normalized gut barrier function, which became completely impervious to the passage of the fluorochrome-labeled dextran compound. Barrier function was normalized in both male and female mice, whether induced by the RG2 strain (p=0.03), or the Lupus S107-48 or Lupus S47-18 strains (p=0.01 for each) (FIG. 9C and FIG. 9D), compared to control mice (FIG. 9E). As larazotide is known to act locally to decrease tight junction (TJ) permeability by blocking zonulin receptors to promote TJ assembly and actin filament rearrangement (Gopalakrishnan et al., 2012), these findings document that gut colonization with Lupus RG strains results in intestinal permeability, with associated immune responses to microbial and nuclear antigens, which can be specifically inhibited by an agent with a known molecular pathway, known to contribute to celiac disease and inflammatory bowel disease (IBD) pathogenesis (Lee, 2015).

Whereas recent studies have demonstrated intestinal RG blooms in a subset of patients with active Lupus nephritis that is temporally associated with disease flares, the current studies investigated the potential in vivo pathogenic properties of RG. As RG is common throughout healthy populations, albeit at lower levels, adverse local effects of intestinal RG colonization on the host may reflect quantitative expansions within intestinal communities and/or arise from genomic variations of these different RG strains. Importantly, diverse RG strains were able to colonize the murine gastrointestinal (GI) tract, including after neonatal RG colonization that was likely mediated by the coprophagic behaviors of the litters from colonized breeding pairs. In part because adult mice raised under GF conditions are known to have associated developmental immunodefects (reviewed in (Round and Mazmanian, 2009)), this finding was especially important as it enabled the study of mice that were neonatally colonized by a natural approach.

The present experimental design enabled comparisons of the influences of RG strains isolated from healthy donors, with strain(s) isolated from Lupus patients. To facilitate quantification of the level of RG colonization, a previously reported RG species-specific genomic qPCR assay was applied. This method was adopted as different strains may differ in efficiency of in vitro culture, which could affect quantitation of recovered colony forming units (CFU), albeit a 16S rDNA-specific method was adopted that cannot discriminate between viable and no longer viable bacterial cells. The methodologic approach disclosed herein allowed for exploration of an enigma highlighted by studies of murine colonization by Enterococcus gallinarum (E. gallinarum), which found small intestinal colonization in the absence of detectable E. gallinarum in the fecal pellets (Manfredo Vieira et al., 2018). In the present studies, at late timepoints after colonization, individual mice with persistent substantial level of RG colonization based on RG detection in cecal luminal contents, at times had no detectable levels RG in matched fecal samples. These findings provide a nuanced technical insight into the limits of 16S rDNA gene amplicon analysis of fecal samples which has become a widely accepted as an unbiased approach to gut microbiota community analysis.

The present studies demonstrated that colonization with some RG strains, which included CC55_001C (RG2) as well as the S107-48 and the S47-18 Lupus strains, reproducibly induced enhanced gut permeability. By contrast, no permeability was induced by RG1 that did not express a lipoglycan, and this benign influence of RG1 strain, from a healthy donor, could be linked to the reported anti-inflammatory properties of the capsular polysaccharide that this strain produces (Henke et al., 2021). As emerging evidence suggests that subclinical disturbances in gut-barrier function are common in lupus patients (Azzouz et al., 2019; Ogunrinde et al., 2019), the current findings suggest that lipoglycan-expressing RG strains may be direct causes of increased intestinal permeability that contribute to a feed-forward inflammatory autoimmune condition in genetically predisposed individuals, which contributes to the loss of tolerance.

Although there was no consistent pattern of sex-biased initial intestinal colonization, female mice consistently demonstrated higher levels of intestinal permeability, whether a consequence of colonization of previously GF mice, neonatal colonization, and/or of antibiotic-conditioned mice raised under SPF conditions. Indeed, women have been shown to be more at risk of intestinal leak (Ogunrinde et al., 2019). These findings may be especially relevant to the role of RG blooms during Lupus pathogenesis, a condition that affects nine-fold more women than men. Recent reports have shown that a major subset of female Lupus patients, as well as their first-degree female relatives, have subclinical abnormalities attributed to breaches in the gut barrier (Azzouz et al., 2019) (Ogunrinde et al., 2019). It is possible that the same mechanisms revealed from the murine colonization studies disclosed herein could be responsible for such findings in patients. The RG lipoglycan shown to contain pathogen-associated molecular patterns (PAMPs) responsible for pro-inflammatory Toll-like receptor (TLR)-agonistic properties was not specifically tested (Azzouz et al., 2019). While the lipoglycan could have directly induced increased intestinal permeability, there could be other RG factors that are (co-)responsible. RG translocation, documented as deposition of RG DNA in the mesenteric lymph node and by the induction of system anti-LG antibodies, may be due to leakiness of the small intestine or another portion of the gut.

Impairments of intestinal barrier function correlated with raised serum levels of zonulin, the only known physiological regulator of intestinal intracellular tight junctions (Sturgeon and Fasano, 2016). While first discovered in studies of gluten enteropathy, intestinal bacteria (including both pathogens and certain commensals) have also been identified as stimuli that can trigger the release of zonulin (Fasano, 2012). Functional intestinal barrier abnormalities induced by multiple RG strains correlated with raised serum zonulin levels, and autoantibody production. The abnormal results of the FITC-dextran assays were completely normalized by oral administration of the specific zonulin receptor antagonist, the octapeptide larazotide that was originally identified from studies of the zonula occludens toxin (ZOT) secreted by Vibrio cholera (Gopalakrishnan et al., 2012). These studies provide the first proof-of-principle for treating Lupus patients with an agent that heals the leaky gut.

Below are the methods used in Examples 1-7 described above.

Generation of an RG-specific lipoglycan specific murine monoclonal antibody. Fecal samples from two lupus nephritis patients (termed S47 & S107), obtained at time of disease flare, were streaked onto bacterial media plates. Based on morphology and growth characteristics, individual colonies were selected, and screened by 16S rDNA gene-specific PCR amplification and DNA sequences used to assign to the Ruminococcus gnavus (RG) species, which was confirmed by whole genome sequence determination and analysis. By this approach, the Lupus-derived RG strains, S47-18 and S107-48 were isolated and whole genomes were later determined.

To generate RG-specific monoclonal antibodies, 10 BALB/c mice were immunized with extract of the (CC55_001C), termed RG2 strain, emulsified in complete Freund's adjuvant and then boosted with lipoglycan (LG) purified from the Lupus S47-18 strain emulsified in incomplete Freund's adjuvant. The spleen from the mouse with the strongest post-immunization was fused with the NS-1 myeloma cells. After subcloning, the spent supernatants were evaluated for IgG-reactivity, which demonstrated highly correlated reactivity with whole extracts of the immunizing RG strain and purified RG lipoglycan, with the subcloned hybridoma cell lines, termed mAb 36.2.2. By immunoblot, this mAb was reactive with 20-25 kilodalton oligobands, equivalent to Lupus serum IgG-reactivity in the lipoglycan producing RG strains, RG2, S107-48 and S47-18 while these bands are absent from the RG1 strain.

Mice. Germ-free (GF) mice were bred and maintained in the gnotobiotic facility at the NYU Langone (Bhattarai and Kashyap, 2016). All mice were C57BL/6 genotype, and locally bred or purchased from Charles River Laboratories (Wilmington MA) and were received at 6-8 weeks of age, or locally bred. All other mice were maintained in specific-pathogen-free (SPF) cages, with free access to food and water. To avoid possible cross-contamination, mice colonized with each of the different strains were separately raised in isolator cages. Mice were housed under a 12-hour light/dark cycle at 23° C.

Intestinal colonization with RG strain. Individual RG strains were streaked, then individual colonies grown in 5 ml of BHI media (Anaerobe Systems) under anaerobic conditions overnight. GF C57BL/6 mice (4 male and 4 female) were colonized by oral gavage of different RG strains (RG1, RG2 and S107-48) of 108 CFU in 500 μL of sterile PBS, every other day for a total of five times. Individual mice were weighed, then fecal pellets and bleeds were collected from individual mice prior to gavage, and again at days 7, 14, and 21 following gavage. Pellets were stored at −80° C. until DNA extraction. The bacterial translocation and burden of R. gnavus in feces was determined by RG-specific qPCR at the indicated time points. All breeding airs yielded litters that were then housed under SPF conditions.

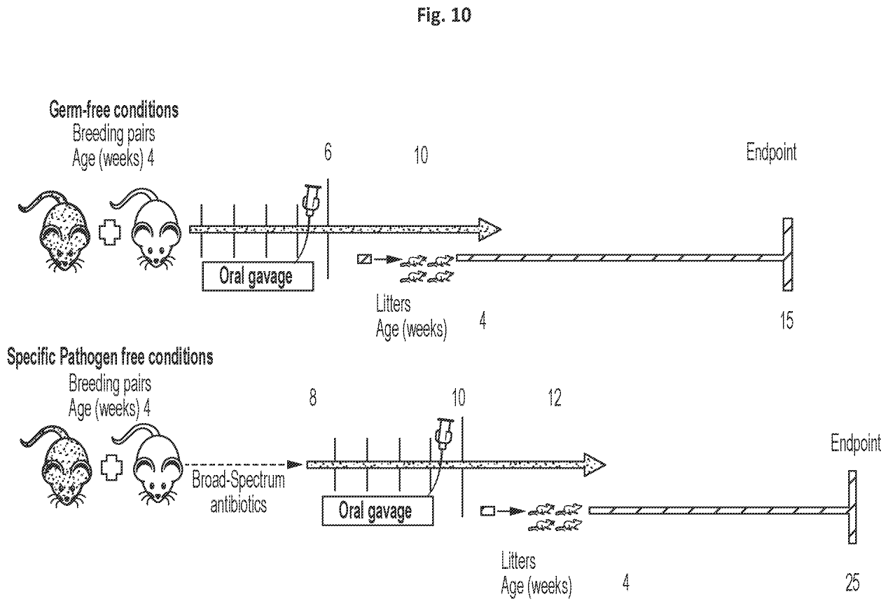

To colonize mice previously raised under Specific Pathogen-Free (SPF) conditions, 4-6-week-old mice were preconditioned with oral antibiotics, then breeding pairs were gavaged with RG1, RG2, or Lupus strains S107-48 or S47-18, and only the pair colonized with the RG1 strain did not yield litter(s). The antibiotic cocktail was composed of vancomycin (0.5 g/L) (Fisher Scientific), neomycin (lg/L) (Fisher Scientific), ampicillin (lg/L) (Fisher Scientific) and metronidazole (lg/L) (Fisher Scientific), with solutions freshly prepared each week in autoclaved drinking water; all antibiotics remained soluble at this concentration. Antibiotics were provided in 100-ml clear glass sippers (Braintree Scientific, Inc., Braintree MA). SPF mice received systemic antibiotics (see above) at 4 weeks of age for one month. Fecal pellets were collected prior to and following antibiotic exposure at days 21, and then weekly until at least a 100-fold decrease in total 16S rDNA gene (representing bacterial burden) in fecal pellets from each mouse was confirmed by qPCR. Mice were then switched to plain water ad libitum for 24 hours. Mice then received the same as above-described oral gavage of different RG strains (RG1, RG2, S107-48 and S47-18) (see FIG. 10).

Quantitative PCR analysis. For bacterial genomic 16S rDNA gene quantitation, DNA was isolated from mice fecal pellets, cecum contents, spleens and mesenteric lymph nodes (MLNs) using QIAamp DNeasy Powersoil or Blood & Tissue kit (Qiagen), according to the manufacturer's instructions. DNA isolated from each of these sample was quantified on a Nanodrop 1000 (Thermo Scientific) and then run for quantitative PCR assays on the StepOnePlus™ Real-Time PCR System (Applied Biosystems) with the Power SYBR Green Master Mix (Applied Biosystems). For end-point PCR reactions, a thermal cycler (Applied Biosystems) was programmed to amplify bacterial DNA using the below-listed primers, which were also used for qPCR experiments. PCR reactions were run with the following conditions: holding stage at 95° C. for 5 minutes followed by 50 cycles of 95° C. for 15 seconds, 58° C. for 30 seconds; and followed by extension at 72° C. for 5 minutes. Total bacterial 16S rDNA gene content was assessed with the oligonucleotide primers.

UniF340
(SEQ ID NO: 17)
(5′-ACTCCTACGGGAGGCAGCAGT-3′)
UniR514
(SEQ ID NO: 18)
(5′-ATTACCGCGGCTGCTGGC-3′).

The RG species-specific 16S rDNA was determined with previously reported oligonucleotide primers (Png et al., 2010):

Fwd
(SEQ ID NO: 19
5′-GGACTGCATTTGGAACTGTCAG-3′
Rev
(SEQ ID NO: 20)
5′-AACGTCAGTCATCGTCCAGAAAG-3′.

In vivo assay of intestinal permeability. To assess intestinal permeability, after a 4-hours fasting from food and water, mice were orally gavaged with 4,000-Da fluorescein (FITC)-dextran (FD4) (Sigma-Aldrich, St. Louis, MO) (250 mg/kg body weight) in doses of 200 μl, and blood was collected 3 hours later. The concentration of the FD4 was determined using a fluorimeter with an excitation wavelength of 490 nm and an emission wavelength of 530 nm. To assess concentration, FD4 in serum was then serially diluted to establish a standard curve.

Leaky gut recovery studies. Zonulin antagonist, larazotide acetate (known as AT-1001, or INN-202), was purchased from BOC Sciences, NY. Individual neonatally-colonized littermates from SPF mice, colonized with different RG strains, were retested after FD4 challenge, and those with abnormal levels then each received 0.15 mg/ml of the zonulin antagonist in drinking water, which was refreshed every day, for 10 consecutive days. After 24 hours rest, intestinal permeability was then retested.

Assays of antigen-reactive IgG antibodies. A custom multiplex bead-based array for the Magpix platform (Luminex, Austin TX) was created by coupling a variety of highly purified thymic native DNAs, purified cell wall lipoglycan (LG) from the RG2 strain (i.e., IgG anti-RG2 LG (formerly termed LG3) (Azzouz et al., 2019)), and the Lupus RG strain S47-18 (i.e., IgG anti-S47-18 strain LG), recombinant S. aureus proteins, endotoxins, and other bacterial antigens and control ligands, to individual microspheres, adapting the manufacturer's protocol and previous reports (Pelzek et al., 2017; Pelzek et al., 2018b; Radke et al., 2018). For antigen-reactive IgG detection, 1,000 microspheres per analyte per well were premixed, sonicated, and then diluted with addition of 100 μl of serum, as indicated. For ELISA bound IgG antibodies were detected with Fc-gamma-specific anti-murine IgG HRP (eBioscience, San Diego CA), and for multiplex bead-based assay with anti-mouse IgG (Fc-specific) F(ab′)2 PE (Jackson ImmunoResearch, West Grove PA). Data were acquired on a Magpix instrument (Luminex) and reported as mean fluorescence intensity (MFI) values, as previously described (Pelzek et al., 2017; Pelzek et al., 2018a; Radke et al., 2018). Plasma zonulin were quantitated with a commercial ELISA Kit. (MyBioSource, San Diego CA.).

Biostatistics. Comparisons were with two-tailed unpaired or paired t-tests, or Spearman correlations, as indicated, with Prism version 9.0 for Mac OS (Graphpad, San Diego CA). p<0.05 was significant.

### Example 8. Gut Dysbiosis in Lupus Patients with High Disease Activity and Active Renal Involvement

The interconnected multidimensional commensal microbial communities harbored within individuals provide a myriad of functions critical for both nutrition and general health. Such commensals also serve as early sparring partners, essential for the development and maintenance of the most fundamental layers of cellular defenders from the innate and adaptive immune systems. In health, there are complex relationships within these microbiota communities that are believed to maintain and reinforce a dynamic equilibrium. However, there is also mounting evidence that imbalances, termed dysbiosis, within gut commensal communities are common in patients with diverse inflammatory and autoimmune diseases (reviewed in (Rosser and Mauri, 2016)). Such imbalances are postulated to unleash the pathogenic potential of inherited susceptibility factors that otherwise remain dormant in unaffected relatives.

Lupus is the archetypic systemic autoimmune disease that carries great morbidity and mortality. Much is known about the immunologic pathways associated with pathologic autoimmunity and tissue injury. Yet despite progressive advances in the therapeutic armentarium and even with regular clinical evaluations, many patients nonetheless suffer from inadequate clinical responses and recurrent flares of disease (Kim et al., 2017). Amongst the greatest morbidity is that incurred by renal involvement that affects up to 60% of patients, with recent reports estimating that 20% will continue to progress to end-stage renal disease (discussed in (Almaani et al., 2017)).

In cross-sectional studies of the gut microbiomes of Lupus patients, communities within an urban female cohort were characterized with great heterogeneity of clinical features and organ system involvement (Azzouz et al., 2019). From these cross-sectional studies, direct correlations were documented between SLE disease activity index (SLEDAI) and dysbiotic shifts associated with reduced community diversity and richness. Many clinically active patients also had gut expansions of Ruminococcus gnavus (RG), a species reassigned to the genus Blautia in the Lachnospiraceae family of spore-forming obligate anaerobes of the Clostridium cluster XIVa of the phylum Firmicutes (Liu et al., 2008), which is one of the 50 most found species in the intestine, albeit with low abundance levels of ˜0.1% in healthy adults (Qin et al., 2010). Notably, in a large untreated Lupus cohort in China, RG was also recently described as “the most discriminative species enriched in the gut microbiota associated with Lupus Nephritis (LN)” (Chen et al., 2020). This suggests broader relevance of RG expansions to Lupus pathogenesis across diverse epidemiologic populations, ethnicities and geography (Chen et al., 2020). Yet, there are few instances in which individual gut species have previously been implicated in clinical autoimmune pathogenesis. It was hypothesized herein that both quantitative changes (i.e., blooms), as well as genomic and phenotypic differences in RG strains, may contribute to Lupus pathogenesis.

Within the current studies, the temporal dynamic stability of the Lupus gut microbiota community was investigated. For this purpose, a collection of fecal samples from Lupus patients and healthy control (CTL) subjects spanning from several months to years was assembled (see below-described methods, Tables 1-6), which were used to newly generate a series of 16S rDNA amplicon libraries.

For the patients included in the experiments and analyses disclosed herein: Table 1 displays the demographic, clinical and treatment features of the Lupus patients with renal involvement evaluated over time; Table 2 displays the demographic, clinical and treatment features of Lupus patients without renal involvement evaluated over time; Table 3 displays SLEDAI domain scoring in patients with Lupus nephritis; Table 4 displays organ involvements reflected by SLEDAI in non-Lupus nephritis patients; Table 5 displays other medications of patients with Lupus nephritis; and, Table 6 displays medications of patients without Lupus Nephritis (SLEDAI, SLE [Systemic Lupus Erythematosus] disease activity index; Renal ACR, Renal according to American College of Rheumatology [ACR] criteria; un, unknown; MEDs, medications; MMF, mycophenolate mofetil; WBC, white blood cell; dsDNA, double-stranded DNA.

TABLE 1
Demographic, clinical and treatment features of Lupus
patients with renal involvement evaluated over time

| | | | | Collection | | | | | | R. |
|---|---|---|---|---|---|---|---|---|---|---|
| Patient | | | | week | | Renal | Renal | MEDs | | gnavus |
| ID | Age | Ethnicity | Sample | span | SLEDAI | ACR | active | Prednisone | MMF | % |
| S47 | 39 | Asian | 1^{st} | 0 | 8 | 1 | 1 | 0 | 1 | 1.1 |
| | | | 2^{nd} | 256 | 8 | | 1 | 0 | 1 | 2.3 |
| S78 | 38 | White | 1^{st} | 0 | 8 | 1 | 0 | 10 | 1 | 0.4 |
| | | Hispanic | 2^{nd} | 176 | 6 | | 0 | 7.5 | 0 | 0.0 |
| | | | 3^{rd} | 205 | 4 | | 0 | 5 | 0 | 0.2 |
| | | | 4^{th} | 246 | 5 | | 0 | 5 | 0 | 0.0 |
| | | | 5^{th} | 268 | 4 | | 0 | 5 | 0 | 0.1 |
| | | | 6^{th} | 291 | 8 | | 1 | 5 | 0 | 9.5 |
| S89 | 37 | Asian | 1^{st} | 0 | 8 | 1 | 1 | 40 | 0 | 0.2 |
| | | | 2^{nd} | 142 | 6 | | 0 | 2.5 | 0 | 0.5 |
| | | | 3^{rd} | 163 | 8 | | 1 | 5 | 0 | 0.4 |
| | | | 4^{th} | 233 | 10 | | 1 | 0 | 0 | 0.1 |
| S107 | 32 | White | 1^{st} | 0 | 14 | 1 | 1 | 20 | 0 | 1.0 |
| | | Hispanic | 2^{nd} | 38 | 22 | | 1 | 25 | 0 | 3.1 |
| S120 | 37 | African | 1^{st} | 0 | 15 | 1 | 1 | 40 | 0 | 3.9 |
| | | American | 2^{nd} | 215 | 2 | | 0 | 0 | 0 | 0.0 |
| S124 | 35 | White | 1^{st} | 0 | 16 | 1 | 1 | 20 | 0 | 0.0 |
| | | Hispanic | 2^{nd} | 114 | 8 | | 0 | 0 | 0 | 0.0 |
| | | | 3^{rd} | 228 | 10 | | 0 | 5 | 1 | 0.0 |
| S134 | 33 | White | 1^{st} | 0 | 4 | 1 | 0 | 0 | 0 | 0.0 |
| | | | 2^{nd} | 201 | 6 | | 0 | 0 | 0 | 0.0 |
| | | | 3^{rd} | 238 | 6 | | 0 | 0 | 0 | 0.0 |
| | | | 4^{th} | 260 | 6 | | 0 | 0 | 0 | 0.0 |
| S172 | 24 | Asian | 1^{st} | 0 | 12 | 1 | 1 | 0 | 0 | 0.0 |
| | | | 2^{nd} | 34 | 9 | | 0 | 0 | 1 | 0.0 |
| | | | 3^{rd} | 49 | 3 | | 0 | 0 | 0 | 0.0 |
| S202 | 38 | White | 1^{st} | 0 | 16 | 1 | 1 | 60 | 1 | 0.0 |
| | | | 2^{nd} | 57 | 2 | | 0 | 0 | 1 | 0.0 |

TABLE 2
Demographic, clinical and treatment features of Lupus
patients without renal involvement evaluated over time

| | | | | Collection | | | | | | R. |
|---|---|---|---|---|---|---|---|---|---|---|
| Patient | | | | week | | Renal | Renal | MEDs | | gnavus |
| ID | Age | Ethnicity | Sample | span | SLEDAI | ACR | active | Prednisone | MMF | % |
| S49 | 52 | African | 1^{st} | 0 | 2 | 0 | 0 | 0 | 0 | 0.4 |
| | | American | 2^{nd} | 41 | 3 | | 0 | 0 | 0 | 0.1 |
| S61 | 42 | Asian | 1^{st} | 0 | 8 | 0 | 0 | 60 | 0 | 0.6 |
| | | | 2^{nd} | 27 | 11 | | 0 | 3.75 | 0 | 4.7 |
| S188 | 57 | Asian | 1^{st} | 0 | 0 | 0 | un | 0 | 0 | 0.1 |
| | | | 2^{nd} | 24 | 5 | | un | 2.5 | 0 | 0.4 |
| S190 | 34 | White | 1^{st} | 0 | 4 | 0 | 0 | 0 | 0 | 0.0 |
| | | Hispanic | 2^{nd} | 67 | 4 | | un | 0 | 0 | 0.0 |
| S191 | 43 | White | 1^{st} | 0 | 4 | 0 | 0 | 0 | 0 | 0.0 |
| | | Hispanic | 2^{nd} | 54 | 0 | | 0 | 0 | 0 | 0.0 |
| | | | 3^{rd} | 83 | 4 | | 0 | 0 | 1 | 0.2 |
| S198 | 35 | White | 1^{st} | 0 | 6 | 0 | 0 | 0 | 1 | 0.0 |
| | | Hispanic | 2^{nd} | 54 | 0 | | 0 | 0 | 1 | 0.0 |
| S205 | 46 | Asian | 1^{st} | 0 | 4 | 0 | 0 | 0 | 0 | 0.0 |
| | | | 2^{nd} | 31 | 6 | | 0 | 0 | 0 | 0.1 |
| | | | 3^{rd} | 52 | 4 | | 0 | 0 | 0 | 0.0 |

TABLE 3
SLEDAI domain scoring in patients with Lupus nephritis

| Patient ID | Sample | SLEDAI | Involvements in the SLEDAI Score |
|---|---|---|---|
| S47 | 1^{st} | 8 | proteinuria, ↓complement, ↑dsDNA |
| | 2^{nd} | 8 | proteinuria, ↓complement, ↑dsDNA |
| S78 | 1^{st} | 8 | rash, alopecia, ↓complement, ↑dsDNA |
| | 2^{nd} | 6 | rash, pericarditis, ↑dsDNA |
| | 3^{rd} | 4 | rash, ↑dsDNA |
| | 4^{th} | 5 | rash, ↓WBC, ↑dsDNA |
| | 5^{th} | 4 | rash, ↑dsDNA |
| | 6^{th} | 8 | proteinuria, ↓complement, ↑dsDNA |
| S89 | 1^{st} | 8 | proteinuria, ↓complement, ↑dsDNA |
| | 2^{nd} | 6 | pleurisy, ↓complement, ↑dsDNA |
| | 3^{rd} | 8 | proteinuria, ↓complement, ↑dsDNA |
| | 4^{th} | 10 | proteinuria, pleurisy, ↓complement, ↑dsDNA |
| S107 | 1^{st} | 15 | proteinuria, rash, alopecia, ulcers, ↓complement, ↓WBC, |
| | | | ↑dsDNA |
| | 2^{nd} | 23 | arthritis, proteinuria, pyuria, rash, alopecia, pleurisy, |
| | | | ↓complement, ↓WBC, ↑dsDNA |
| S120 | 1^{st} | 15 | myositis, proteinuria, pleurisy, ↓complement, ↑dsDNA, fever |
| | 2^{nd} | 2 | ↑dsDNA |
| S124 | 1^{st} | 16 | visual disturbances, arthritis, ↓complement, ↑dsDNA |
| | 2^{nd} | 8 | arthritis, ↓complement, ↑dsDNA |
| | 3^{rd} | 10 | arthritis, pericarditis, ↓complement, ↑dsDNA |
| S134 | 1^{st} | 4 | ↓complement, ↑dsDNA |
| | 2^{nd} | 6 | rash, ↓complement, ↑dsDNA |
| | 3^{rd} | 6 | rash, ↓complement, ↑dsDNA |
| | 4^{th} | 6 | rash, alopecia, ↓complement |
| S172 | 1^{st} | 12 | hematuria, proteinuria, ↓complement, ↑dsDNA |
| | 2^{nd} | 9 | proteinuria, ↓complement, ↓WBC, ↑dsDNA |
| | 3^{rd} | 7 | proteinuria, ↓WBC, ↑dsDNA |
| S202 | 1^{st} | 16 | hematuria, proteinuria, pyuria, ↓complement. ↑dsDNA |
| | 2^{nd} | 2 | ↑dsDNA |

TABLE 4
Organ involvements reflected by SLEDAI in non-Lupus nephritis patients

| Patient ID | Sample | SLEDAI | Involvements in the SLEDAI Score |
|---|---|---|---|
| S49 | 1^{st} | 2 | ↓complement |
| | 2^{nd} | 3 | ↓complement, ↓WBC |
| S61 | 1^{st} | 8 | rash, alopecia, ↓complement, ↑dsDNA |
| | 2^{nd} | 11 | arthritis, alopecia, ↓complement, ↓WBC, ↑dsDNA |
| S188 | 1^{st} | 9 | arthritis, ↓complement, ↓WBC, ↑dsDNA |
| | 2^{nd} | 5 | ↓complement, ↓WBC, ↑dsDNA |
| S190 | 1^{st} | 4 | ↓complement, ↑dsDNA |
| | 2^{nd} | 4 | ↓complement, ↑dsDNA |
| S191 | 1^{st} | 4 | Arthritis |
| | 2^{nd} | 0 | — |
| | 3^{rd} | 4 | Arthritis |
| S198 | 1^{st} | 6 | arthritis, ↑dsDNA |
| | 2^{nd} | 0 | |
| S205 | 1^{st} | 4 | ↓complement, ↑dsDNA |
| | 2^{nd} | 6 | ↓complement, ↑dsDNA, alopecia |
| | 3^{rd} | 4 | ↓complement, ↑dsDNA |

TABLE 5
Other medications of patients with Lupus nephritis

| Patient ID | Sample | Other medications |
|---|---|---|
| S47 | 1^{st} | hydroxychloroquine 400 mg, azathioprine 150 mg |
| | 2^{nd} | hydroxychloroquine 400 mg |
| S78 | 1^{st} | hydroxychloroquine 400 mg |
| | 2^{nd} | hydroxychloroquine 400 mg, milatuzumab/placebo trial |
| | 3^{rd} | hydroxychloroquine 400 mg, methotrexate 20 mg |
| | 4^{th} | hydroxychloroquine 400 mg, methotrexate 20 mg |
| | 5^{th} | hydroxychloroquine 400 mg, methotrexate 20 mg |
| | 6^{th} | hydroxychloroquine 400 mg, methotrexate 20 mg |
| S89 | 1^{st} | hydroxychloroquine 400 mg, |
| | 2^{nd} | hydroxychloroquine 400 mg, belimumab 10 mg, azathioprinc 100 mg |
| | 3^{rd} | hydroxychloroquine 400 mg, belimumab 10 mg, azathioprine 100 mg |
| | 4^{th} | hydroxychloroquine 400 mg, methylprednisolone 1000 mg |
| S107 | 1^{st} | hydroxychloroquine 400 mg, antifrolumab/placebo study |
| | 2^{nd} | hydroxychloroquine 400 mg |
| S120 | 1^{st} | hydroxychloroquine 400 mg |
| | 2^{nd} | hydroxychloroquine 200 mg, azathioprine 100 mg |
| S124 | 1^{st} | hydroxychloroquine 400 mg |
| | 2^{nd} | hydroxychloroquine 400 mg |
| | 3^{rd} | hydroxychloroquine 400 mg |
| S134 | 1^{st} | hydroxychloroquine 400 mg |
| | 2^{nd} | hydroxychloroquine 300 mg, belimumab 10 mg |
| | 3^{rd} | hydroxychloroquine 300 mg, belimumab 10 mg |
| | 4^{th} | hydroxychloroquine 300 mg |
| S172 | 1^{st} | hydroxychloroquine 400 mg |
| | 2^{nd} | hydroxychloroquine 400 mg |
| | 3^{rd} | hydroxychloroquine 300 mg |
| S202 | 1^{st} | hydroxychloroquine 400 mg, methotrexate 12.5 mg |
| | 2^{nd} | hydroxychloroquine 300 mg |

TABLE 6
Medications of patients without Lupus nephritis

| Patient ID | Sample | Other medications |
|---|---|---|
| S49 | 1^{st} | hydroxychloroquine 400 mg, azathioprine 100 mg |
| | 2^{nd} | hydroxychloroquine 400 mg, azathioprine 100 mg |
| S61 | 1^{st} | hydroxychloroquine 400 mg |
| | 2^{nd} | hydroxychloroquine 400 mg |
| S188 | 1^{st} | hydroxychloroquine 400 mg |
| | 2^{nd} | hydroxychloroquine 400 mg |
| S190 | 1^{st} | hydroxychloroquine 400 mg |
| | 2^{nd} | hydroxychloroquine 300 mg |
| S191 | 1^{st} | hydroxychloroquine 400 mg |
| | 2^{nd} | hydroxychloroquine 200 mg |
| | 3^{rd} | hydroxychloroquine 200 mg |
| S198 | 1^{st} | hydroxychloroquine 400 mg |
| | 2^{nd} | hydroxychloroquine 400 mg |
| S205 | 1^{st} | hydroxychloroquine 300 mg |
| | 2^{nd} | hydroxychloroquine 400 mg |
| | 3^{rd} | hydroxychloroquine 300 mg |

Lupus patients commonly displayed gut microbiota community instability. Over time, within these distorted communities dramatic but ephemeral blooms of several bacterial pathogens were documented. Most notably, gut spikes in the abundance of RG, an obligate anaerobe, arose concordant with significant renal disease activity flares in nearly half of LN patients.

To characterize the features of strains responsible for RG blooms, individual RG colonies were isolated from the fecal samples of two patients at the time of their LN flare, and these RG strains were found to produce a novel immunogenic lipoglycan with common shared structural and antigenic features. The lipoglycan was physically associated with the R. gnavus bacteria, with the blooms occurring during (or that directly cause) disease flares, and this RG lipoglycan was recognized by IgG antibodies secreted into the circulation of LN patients. It was hypothesized herein that these antibodies are integral to the immune complexes that cause disease, tissue damage and especially in glomerular injury (i.e., immune-mediated renal damage that can result in renal failure). These findings provide the first evidence of previously unsuspected features of the intimate relationship between intestinal microbiota dynamic instability, blooms of a pathobiont, and Lupus pathogenesis.

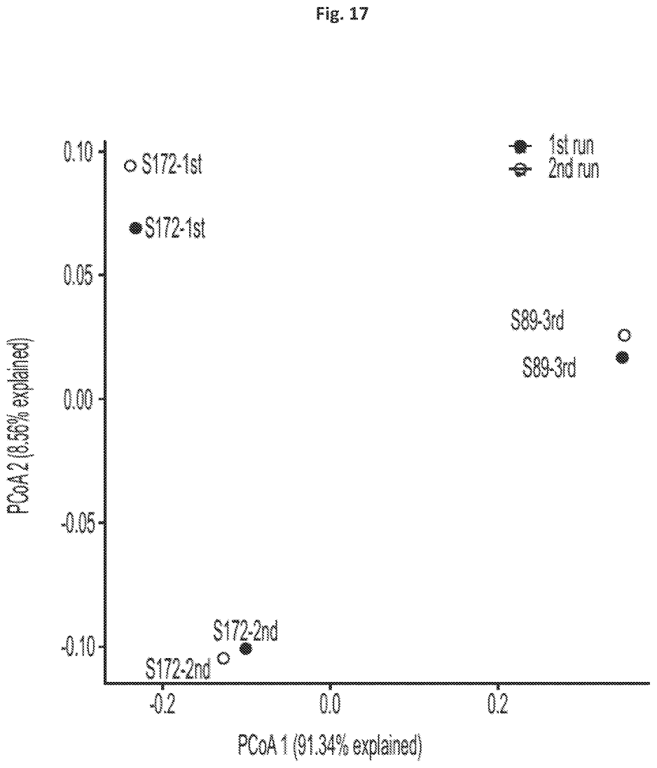

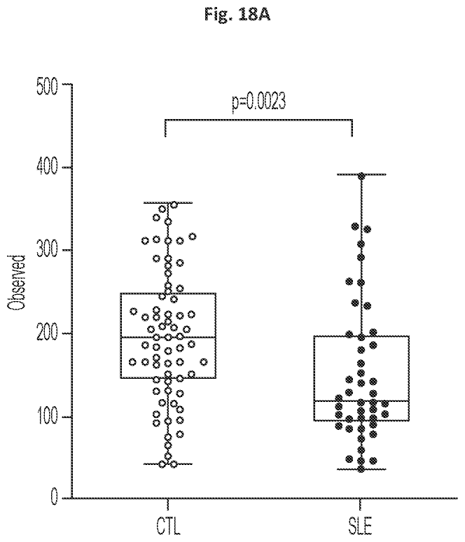

To study the gut microbial communities in sequential samples from disease-affected individuals, two to six fecal samples were examined from 16 individual patients, representing 44 samples obtained over 24-291 weeks following initial recruitment (Tables 1-4). For comparisons, 72 samples from 22 healthy adult female CTL volunteers were also evaluated, which included 2 to 12 samples from 9 individuals. To assess the diversity within these bacterial communities, fecal DNA was extracted and 16S rDNA gene amplicon libraries were generated by targeted amplification of a standard gene fragment. These 114 libraries were sequenced in two batches with consistent results in technical duplicates (FIG. 17). After sequence analysis documented that each library had between 10,345 and 307,479 reads assignable to Amplicon Sequence Variants (ASVs) with known taxonomic associations, which enabled estimations of the relative abundance of diverse taxa in each sampled community.

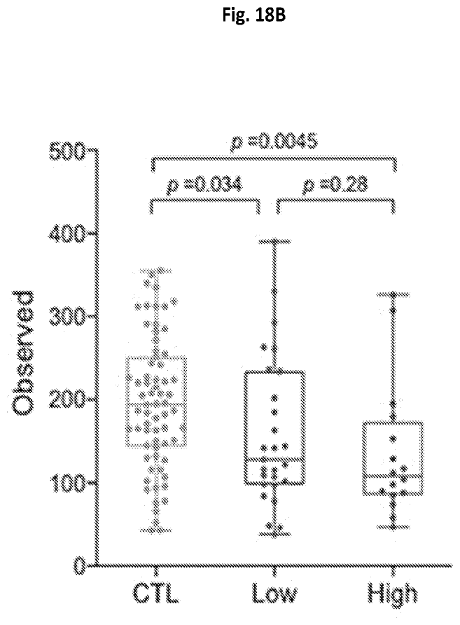

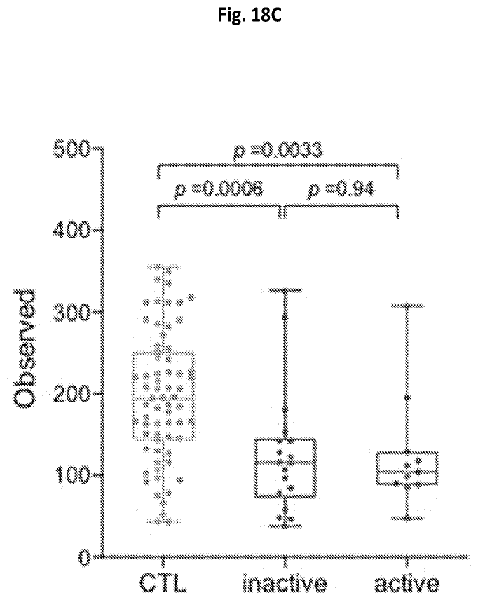

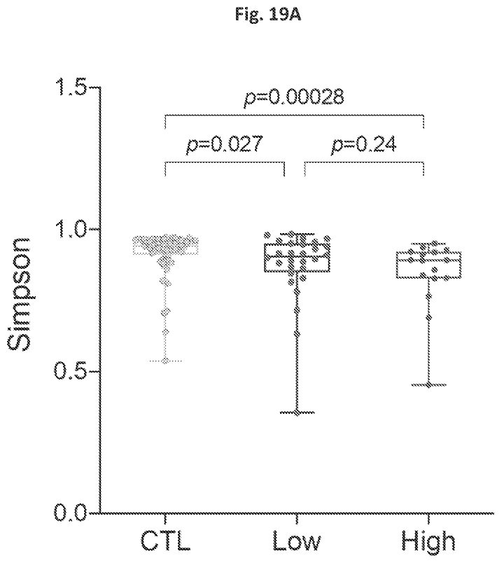

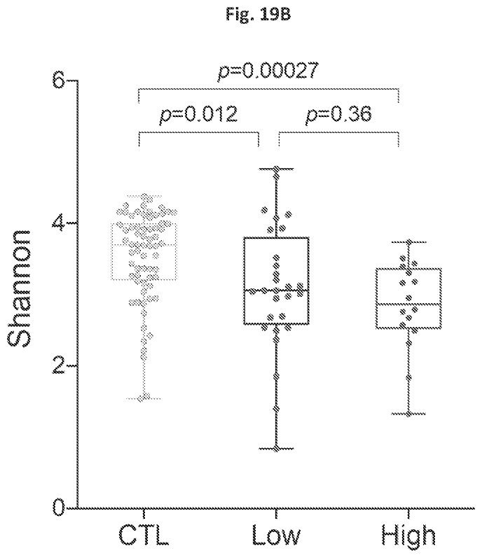

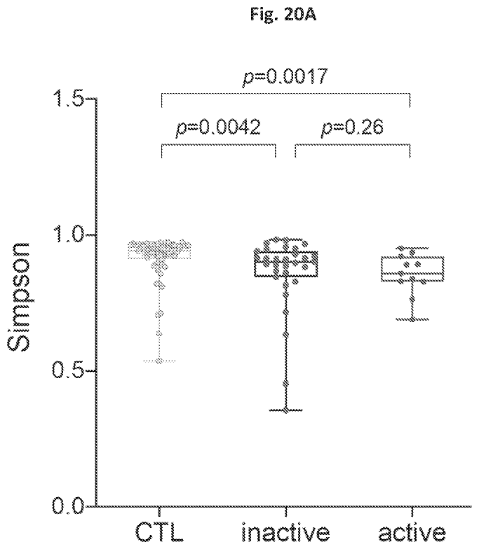

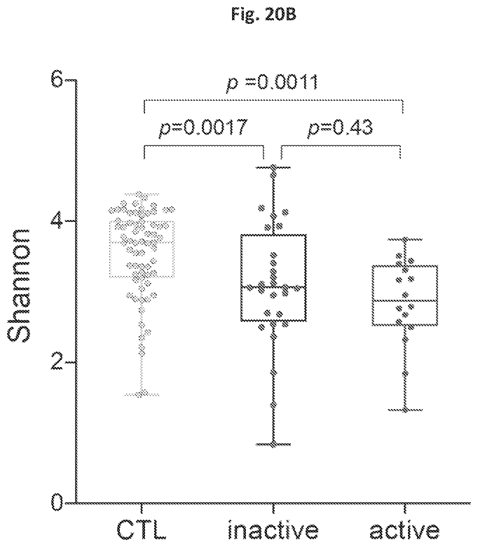

In these analyses, patients who fulfilled ACR criteria for renal involvement at any time (Hochberg, 1997) were here referred to as LN (Table 1 and Table 3), and these patients were then dichotomized based on renal disease activity at the time of sampling as inactive or active, based on urinary protein creatinine ratio (uPCR)<0.5 or >0.5 respectively, as defined in the SLEDAI domain. Others are referred to as non-renal patients (Table 2 and Table 4). Based on earlier microbiome studies (Azzouz et al., 2019), those with overall SLEDAI score of >8 were designated as high disease activity, with others as low disease activity. These gut community findings reiterated earlier reported results (Azzouz et al., 2019) that high SLEDAI scores and active LN were both associated with decreased richness/diversity alpha diversity of microbiota communities, reflected in several measures of richness and evenness in estimates of alpha diversity, compared to CTL subjects (FIGS. 18-21).

### Example 9. Lupus Disease Activity is Associated with Greater Phylogenetic Diversity

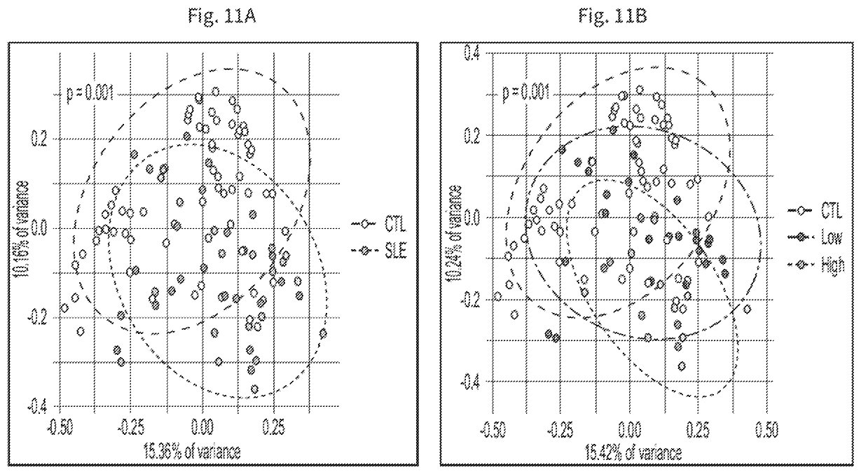

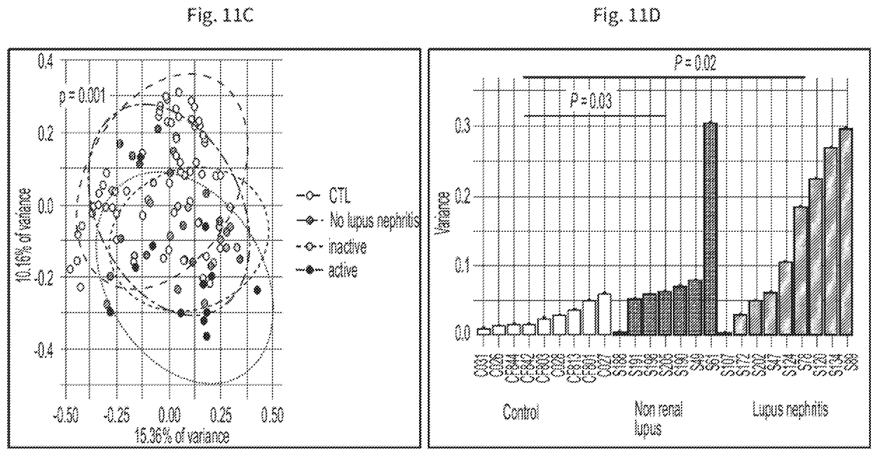

Based on Principal Coordinates Analysis (PCoA), the communities recovered from Lupus patients displayed significant differences overtime in beta diversity compared to CTL using Jensen-Shannon divergence (JSD) dissimilarity metric (multivariant distance Welch's W_{d}* test, p=0.001) (Hamidi et al., 2019) (FIG. 11A). Furthermore, there were significantly greater differences with communities associated with high disease activity (p=0.001) (FIG. 11B), and in communities from patients with active LN (p=0.001) (FIG. 11C).

### Example 10. Instability Over Time is a Common Feature of Lupus Gut Microbiota Communities

Whether variance within the composition of the libraries from individual Lupus and healthy donors was stable over time was next investigated. To assess stability of gut microbiota over time, community-wide multivariate analyses were performed on the multiple libraries from 16 Lupus-affected individuals and 9 healthy subjects, without assumptions based on time-intervals. Using a proven approach for estimating averages within group dissimilarity (Hamidi et al., 2019), limited differences in overall composition amongst the sampled communities from each of the CTL subjects were found, while the level of variance was remarkably variable between communities sampled at different timepoints from the same individual Lupus patient (Kruskal Wallis ANOVA, p=0.03) (FIG. 11D). Compared to controls, there was much greater variance within the Lupus subgroups; non-renal (two-tailed, p=0.03), and renal (LN) (p=0.02) (FIG. 11D). However, no overall correlations were found between community variance with disease duration, the periods between sample collection, the maximal disease activity, the range of disease activity in different visits (Tables 1-4), or the medication regimen in an individual subject (Tables 5-6). Furthermore, the variance in the renal and non-renal groups were not significantly different (p=0.38, NS). Taken together, these findings suggest the gut microbiota communities in Lupus patients are inherently unstable in microbial composition overtime. These observations suggested that further taxonomic investigations were needed, in part to test the hypothesis that within these unstable community milieus, specific bacterial species underwent expansions.

### Example 11. Dynamic Blooms of Individual Bacterial Species are Common within Lupus Gut Microbiota Communities

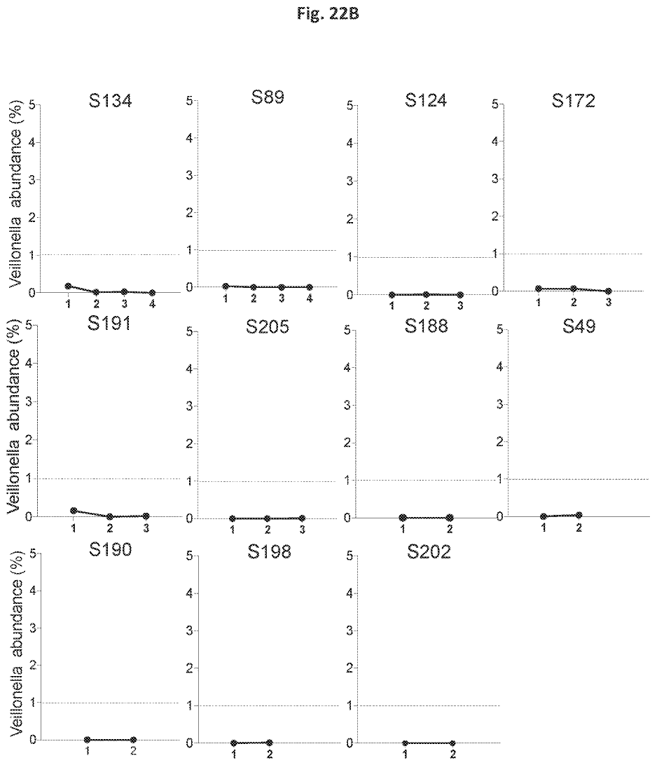

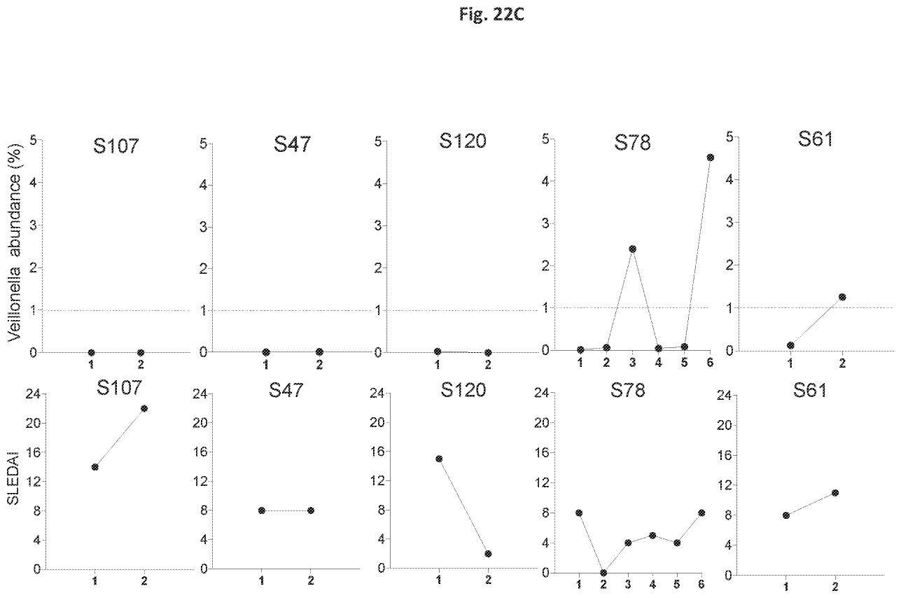

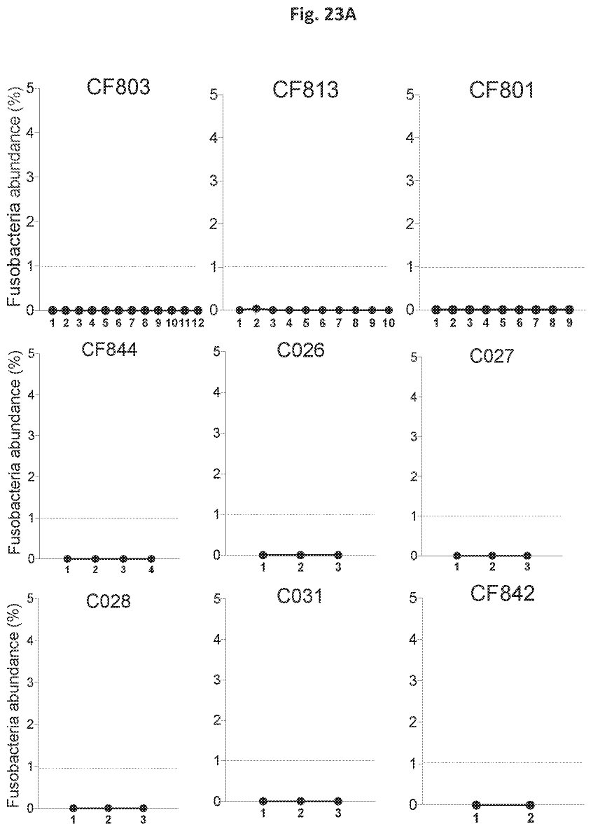

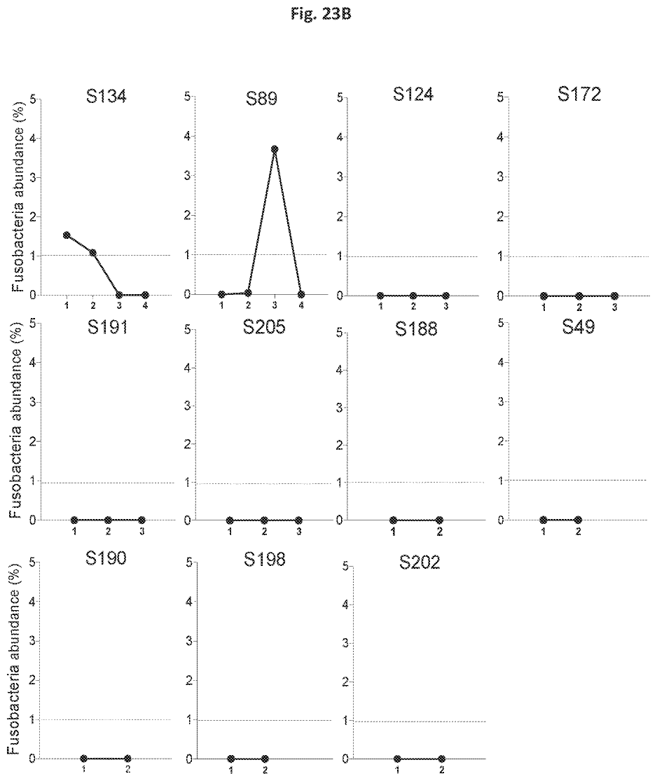

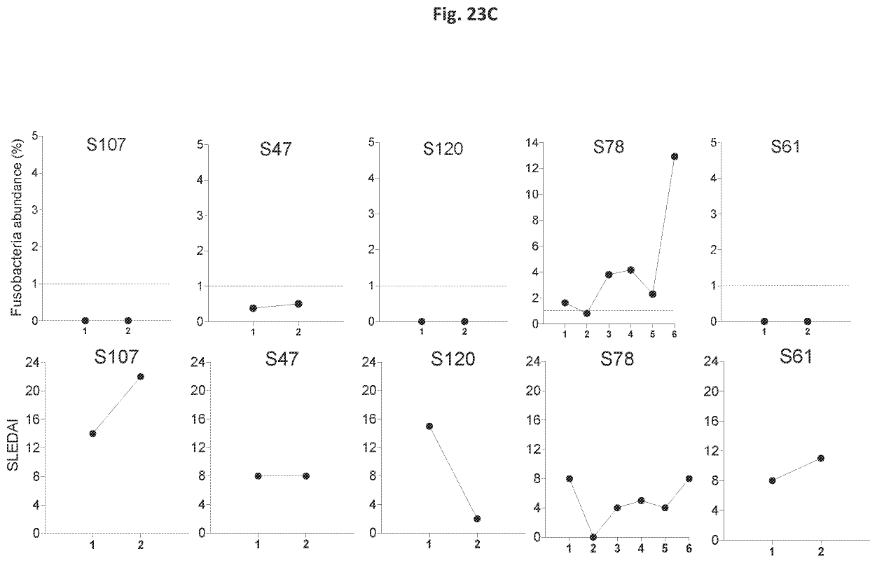

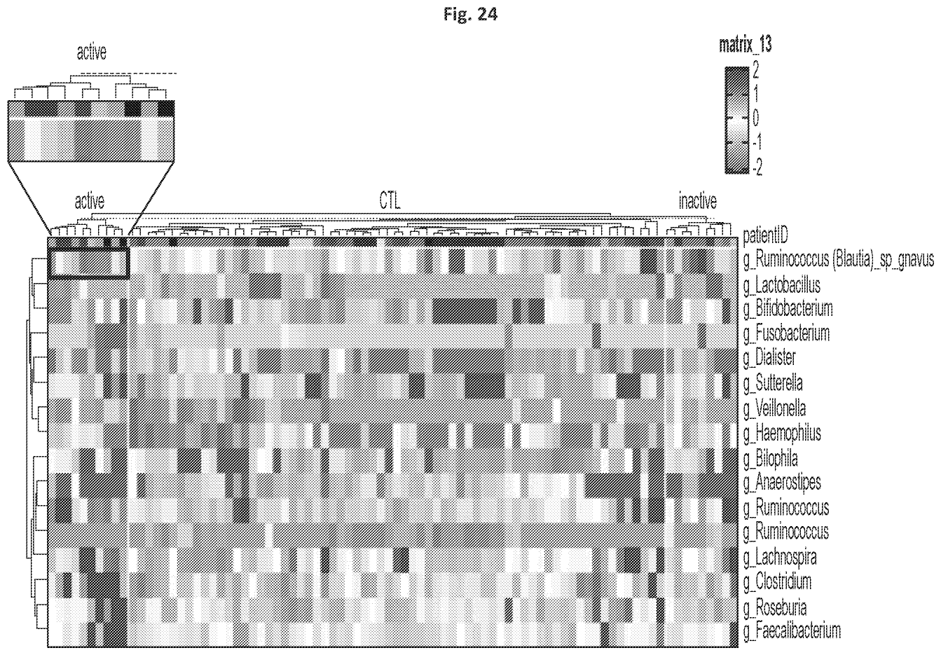

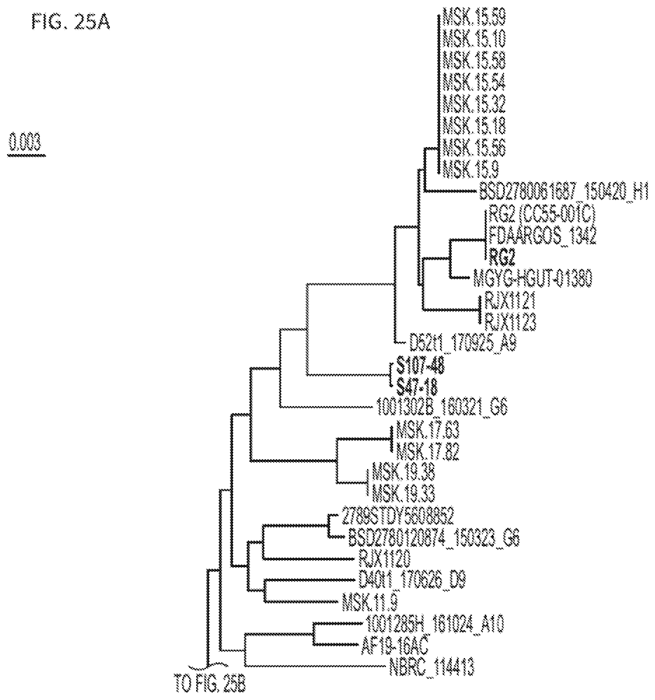

In-depth analyses of the sequential microbiota libraries revealed that in several individual Lupus patients there were transient, but often pronounced, blooms of ASV-defined taxa within both Veillonella and Fusobacterium genera, which were absent in all healthy subjects (FIGS. 22-23). Whereas Veillonella, a Gram-negative Firmicutes genus, can be a normal member of the intestinal or the oral mucosal communities, these can also represent local outgrowths, representing translocations from the oral cavity into the intestine, or even overt infections (Brook, 1996). The Fusobacterium genus of anaerobic gram-negative non-spore-forming bacteria are generally considered oral pathogens (Aliyu et al., 2004). There were no associations between these types of blooms with specific Lupus clinical features or organ involvement, and neither temporal associations between Veillonella nor Fusobacterium blooms nor with overall flares of Lupus disease activity (FIGS. 22-23, Tables 1-4), as shown in taxonomic surveys at a genus and species level (FIG. 24).

### Example 12. RG Blooms were Concordant with Lupus Disease Flare Episodes

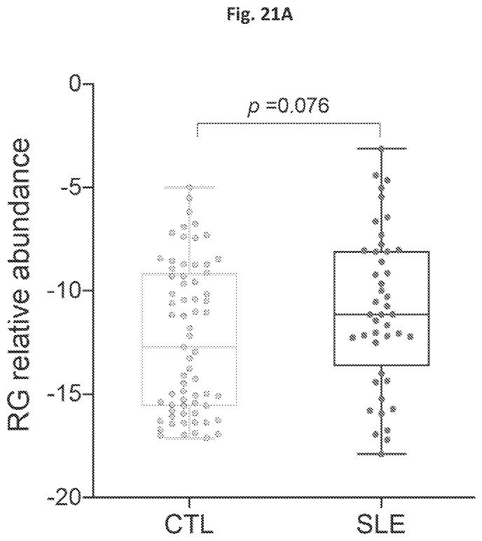

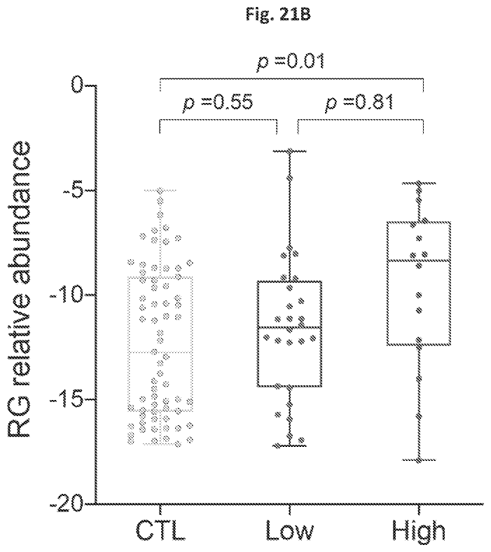

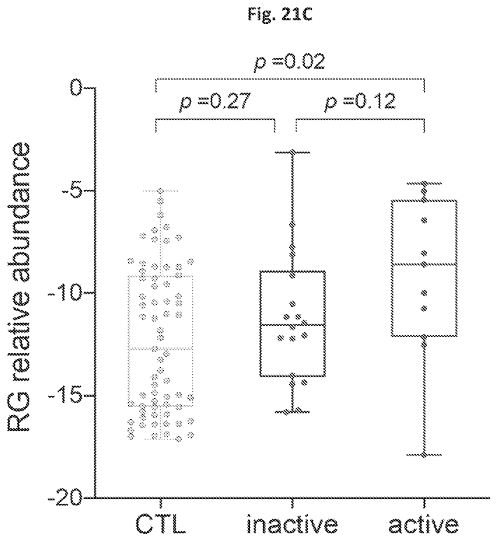

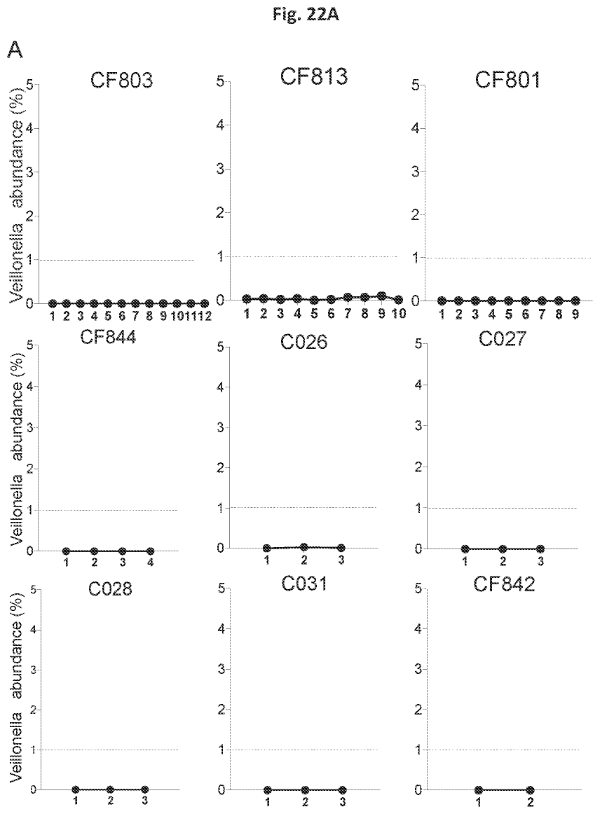

In an earlier cross-sectional report documented a mean 5-fold over-representation of RG within a larger SLE cohort compared with the libraries from controls (Azzouz et al., 2019). In the current studies, quantitative increases in RG relative overall abundance were also found (Wilcoxon, p=0.076) (FIG. 21A). Increases in RG abundance were most marked in patients with high disease activity, with SLEDAI scores of 8 or greater (Wilcoxon, p=0.01) (FIG. 21B), and in patients with active renal disease (Wilcoxon, p=0.02) (FIG. 20 and FIG. 21C, FIG. 24). Furthermore, when examined in a continuous distribution, there was a modest but significant direct correlation between disease activity and RG abundance (Spearman, r=0.320, p=0.034). Overall, these findings therefore reaffirmed earlier associations between RG expansions within Lupus microbiota communities, proteinuria and high disease activity in general.

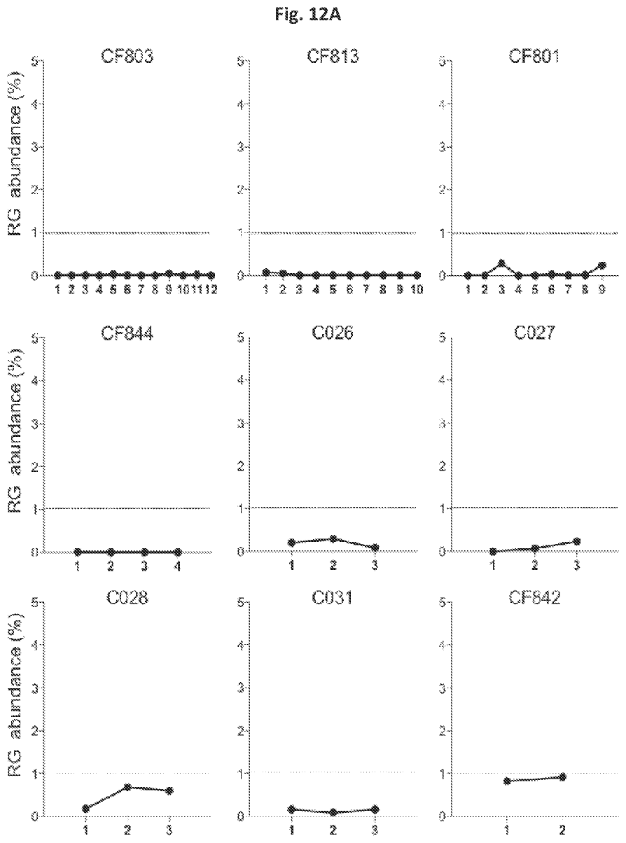

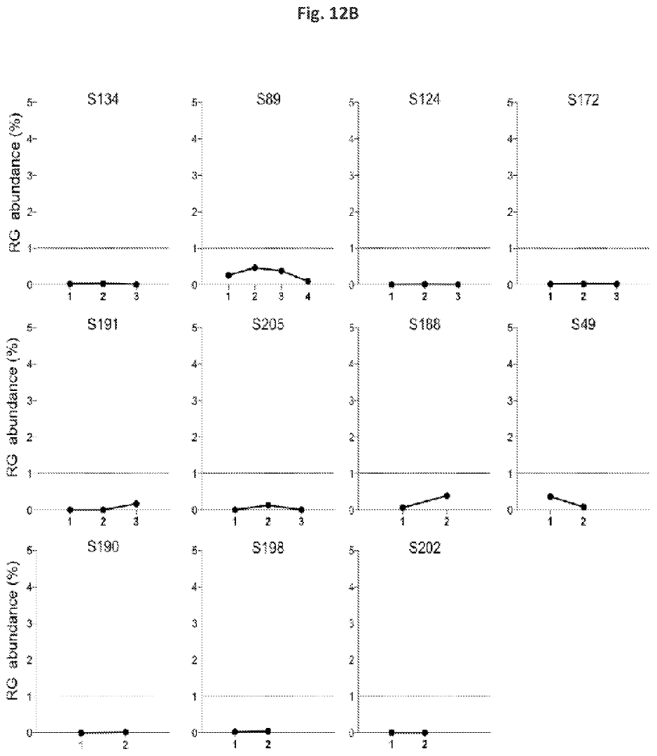

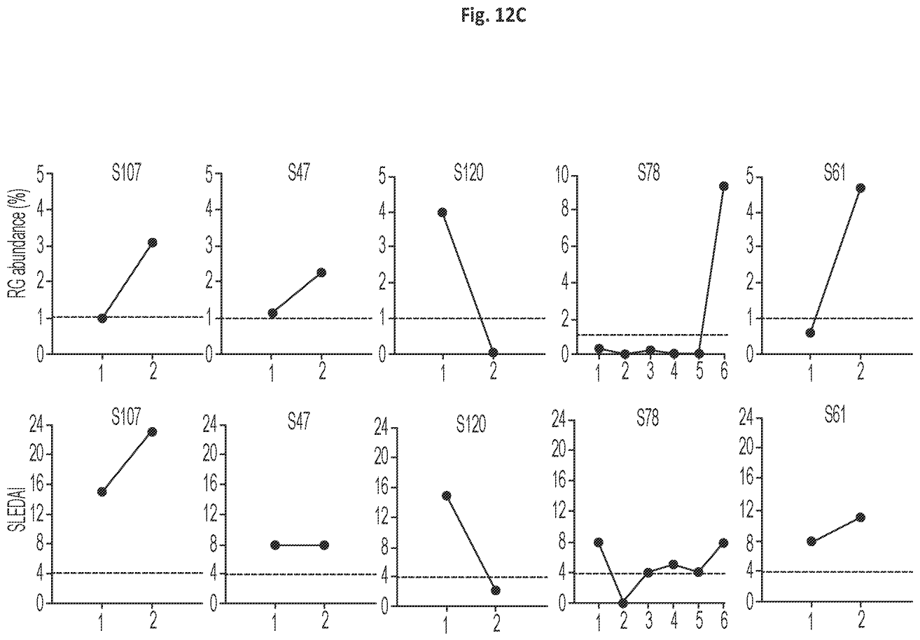

To investigate temporal changes in RG abundance in these patients, the sequential libraries of the CTL and Lupus patients were examined. These surveys included nine LN patients (identified as S47, S78, S89, S107, S120, S124, S134, S172 and S202), and another seven patients who never had renal manifestations (i.e., non-renal patients; S49, S61, S188, S190, S191, S198 and S205) but instead had histories of inflammatory arthritis, cutaneous disease, and/or other disease features (Table 1 and Table 2). In accord with the level reported in a large-scale population-based survey, an average 0.15% RG abundance was observed, with remarkable stability within the healthy female CTLs (FIG. 12A). Amongst the 16 Lupus patients, 11 patients exhibited low or undetectable level of RG abundance in their gut communities, with limited variation overtime in individual patients (FIG. 12B). Of these 11 patients, patient S134, who had quiescent LN during the study (Table 1), also had low or undetectable RG in all four timepoints sampled. Similarly, another five LN patients also displayed stable low RG abundance pattern despite episodes of active renal disease documented at one or more of the sampling timepoints (FIG. 12B).

In contrast, changes in RG abundance were detected in the four other LN patients (˜44% of those studied over time) (S107, S47, S120 and S78). Each patient had an RG bloom that reached a mean of 9-fold higher abundance than in other sample(s) from the same donor. Such an RG bloom, with temporal concordance with clinical disease flare, was also identified in patient (S61) who had non-renal involvement, with inflammatory arthritis (FIG. 12C). Taken together, when only samplings from these 5 patients with an episode of RG bloom were examined, the level of disease activity significantly correlated with the RG abundance (r=0.320, p=0.03). Hence, within individual Lupus patients with unstable gut communities, RG, which colonizes the host intestine with close proximity to the mucosal epithelial barrier (Nava et al., 2011), underwent blooms temporally concordant with disease flares. Thus, RG blooms may contribute to the clinical pattern of relapsing-remitting disease activity that is widely documented to occur in many Lupus patients despite close clinical monitoring and treatment (Barr et al., 1999).

### Example 13. Isolation and Characterization of RG Strains from LN Patients at Time of Disease Flare

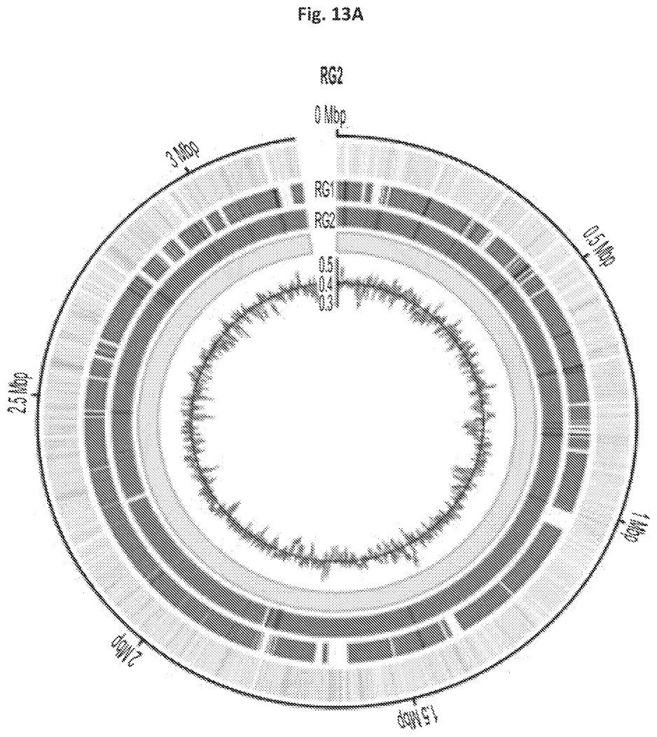

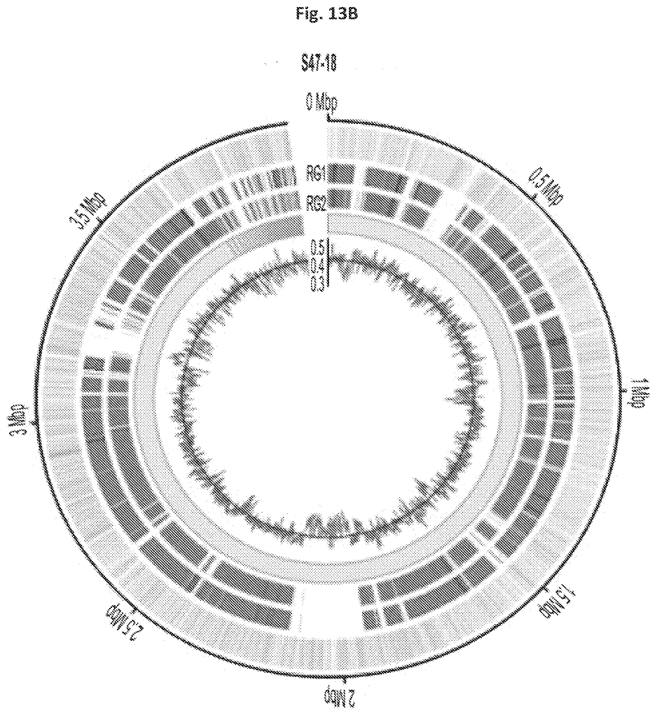

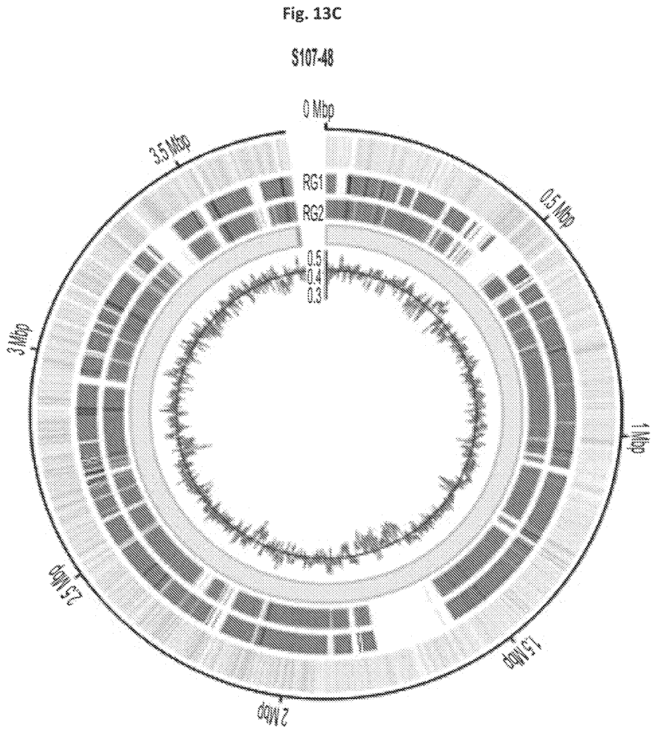

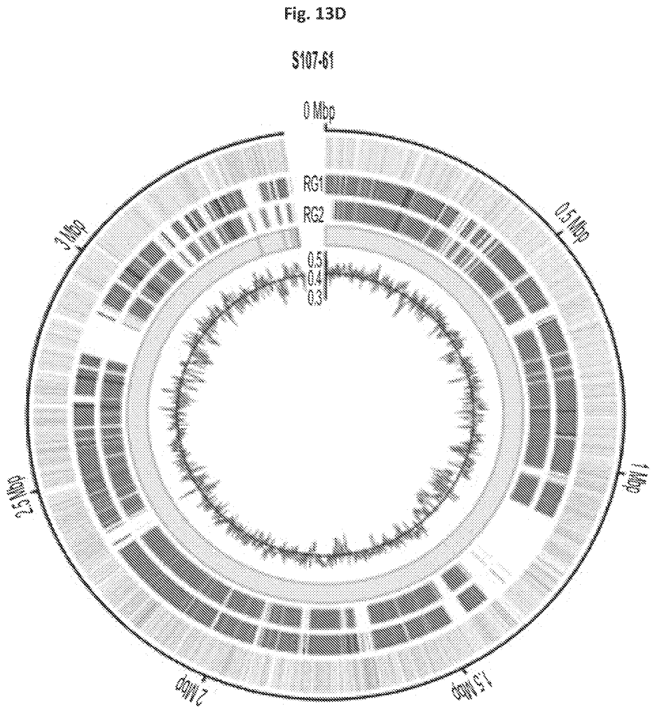

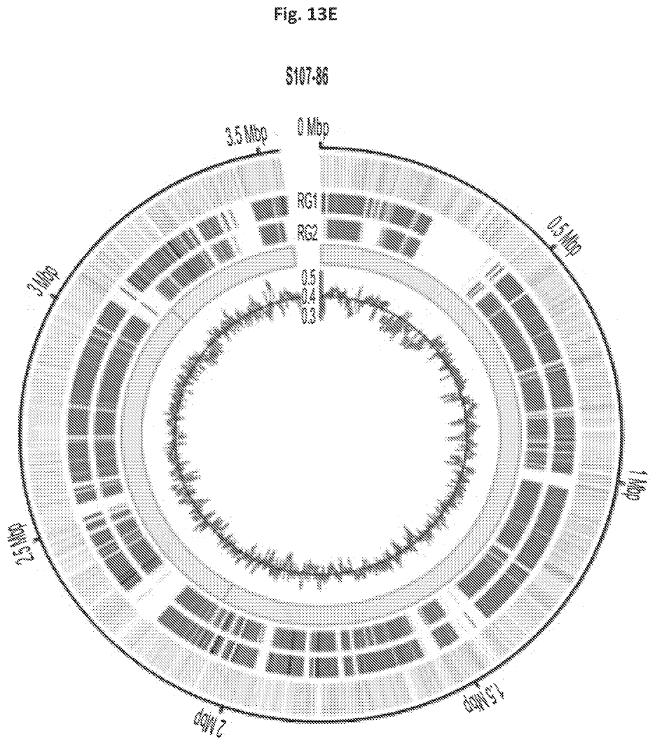

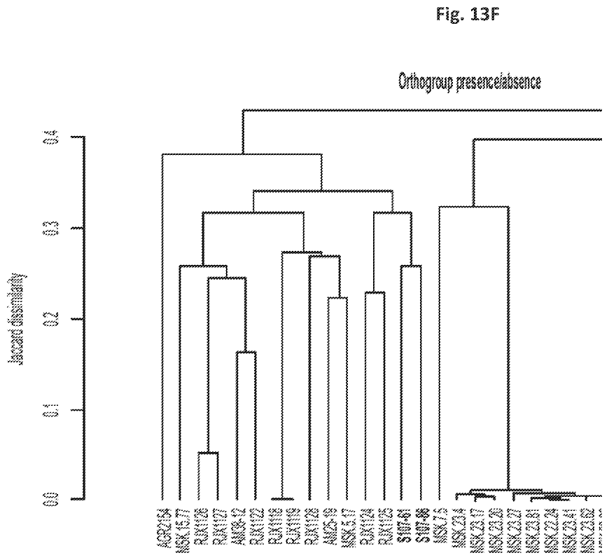

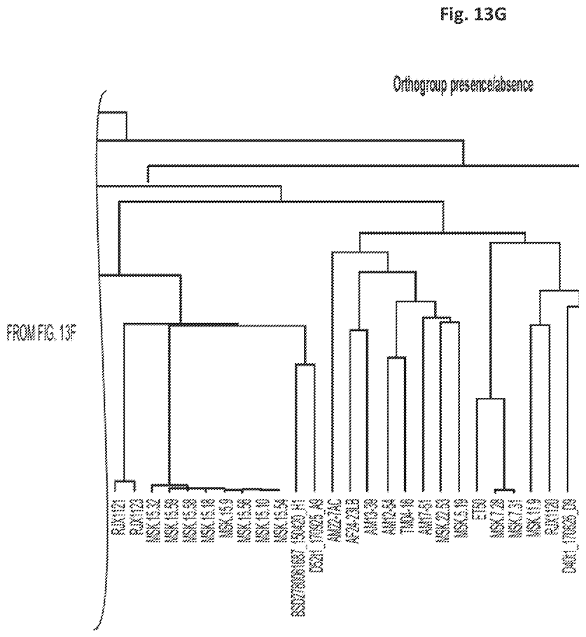

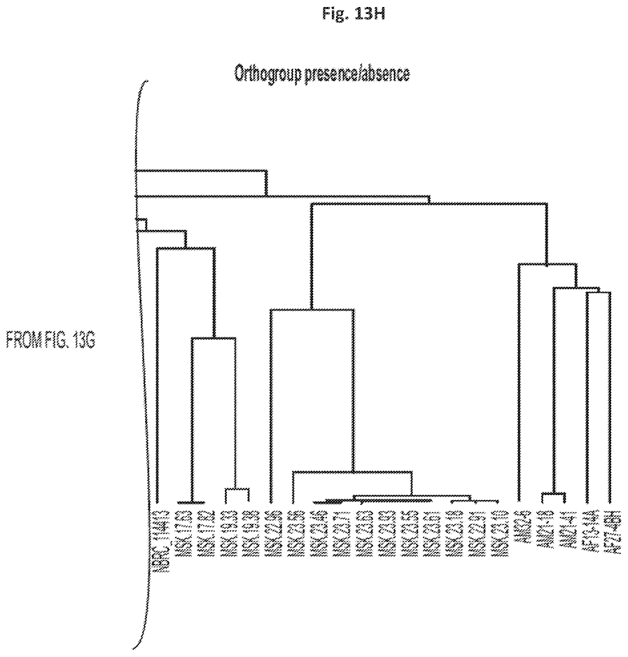

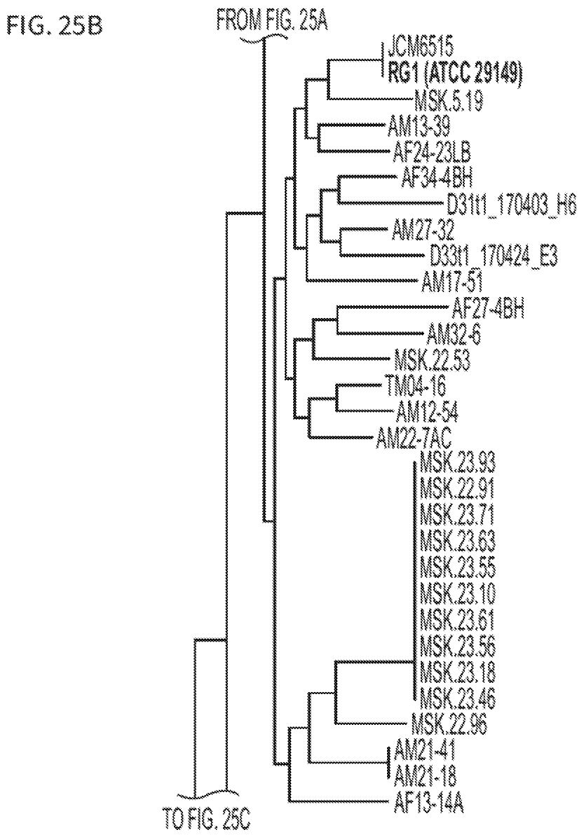

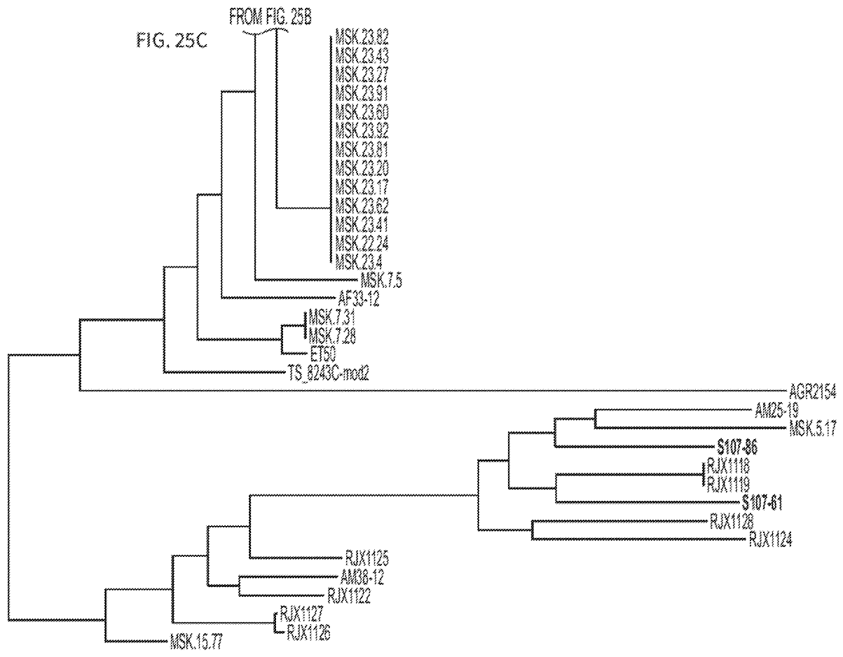

To investigate the genomic relatedness of strains that colonize Lupus patients, the genomes of RG isolates from fecal samples of LN patients were characterized. Whereas selective media for RG in vitro culture are currently unknown, initial efforts using fecal samples from patients at time of low disease activity were nonproductive. However, RG isolates were obtained from fecal samples from two active LN patients, S47 and S107, from the time of LN clinical flares when RG blooms were documented by 16S rDNA amplicon analysis (FIG. 12C). These recovered RG isolates were designated with a prefix accordingly to their donor. Each of these novel whole genome sequences were compared to the RG1 and RG2 type strains (FIGS. 13A-13E), with comparisons to all known RG isolate genomes (FIGS. 13F-13H and FIGS. 25A-25C).

### Example 14. LN Serum Antibodies Recognize Conserved Non-Protein Oligoband Antigens in RG Strains from LN

In an earlier report of serologic cross-sectional surveys of a female Lupus cohort, patients with higher disease activity, and especially those with active renal disease (i.e., Lupus Nephritis, LN), had high-titer serum IgG-responses to ⅛ RG strains originally obtained from healthy donors (Azzouz et al., 2019). The reactivity with this RG type strain, CC55-00C (which is termed RG2) (FIG. 13), was predominantly against a novel lipoglycan (LG), whereas such antibody responses were infrequent or absent in patients with low disease activity and undetectable in other immune-mediated glomerulopathies or healthy female controls (Azzouz et al., 2019).

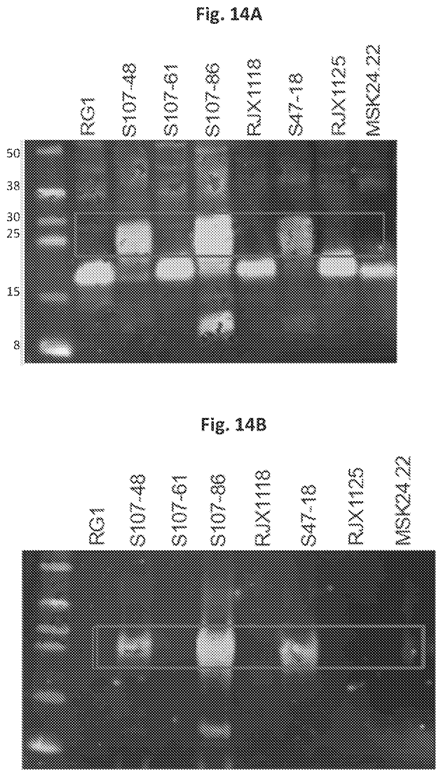

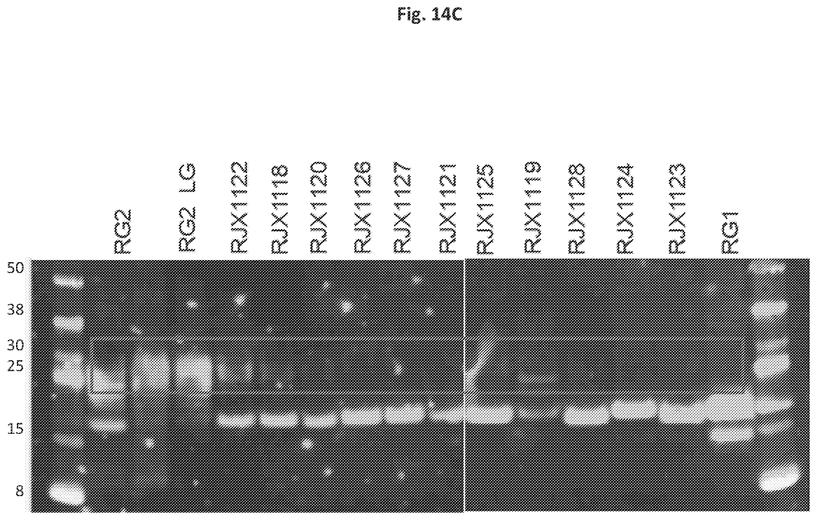

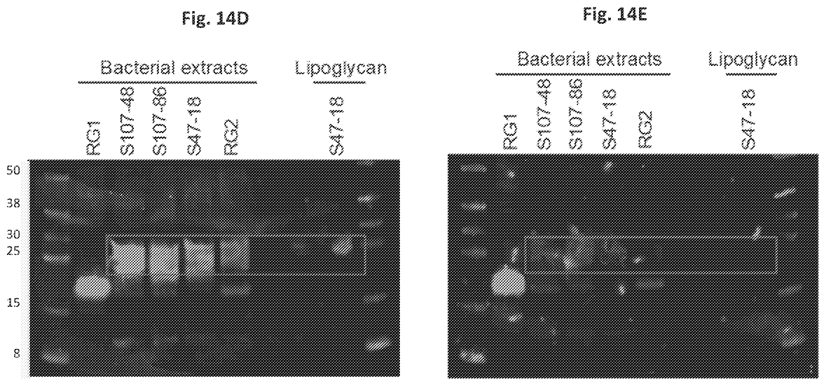

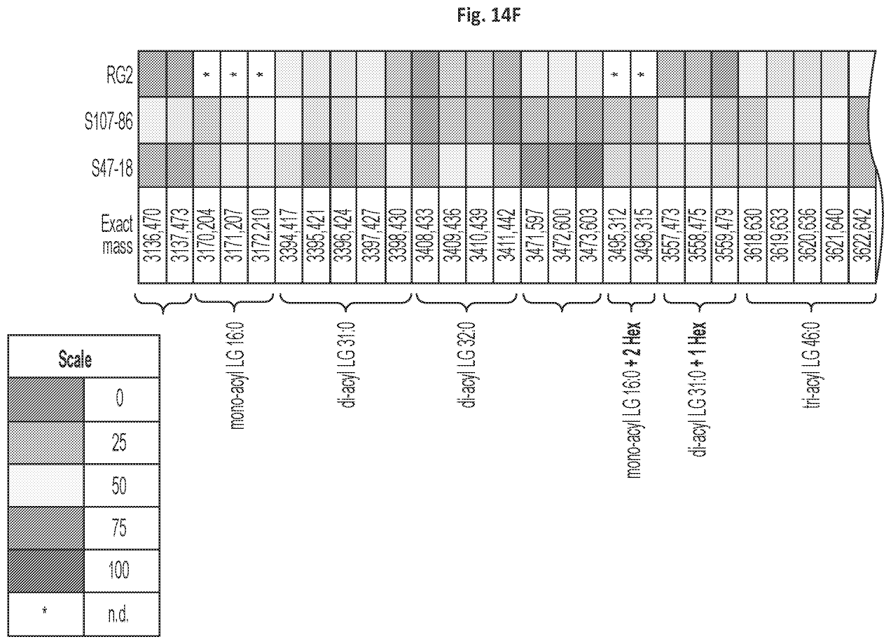

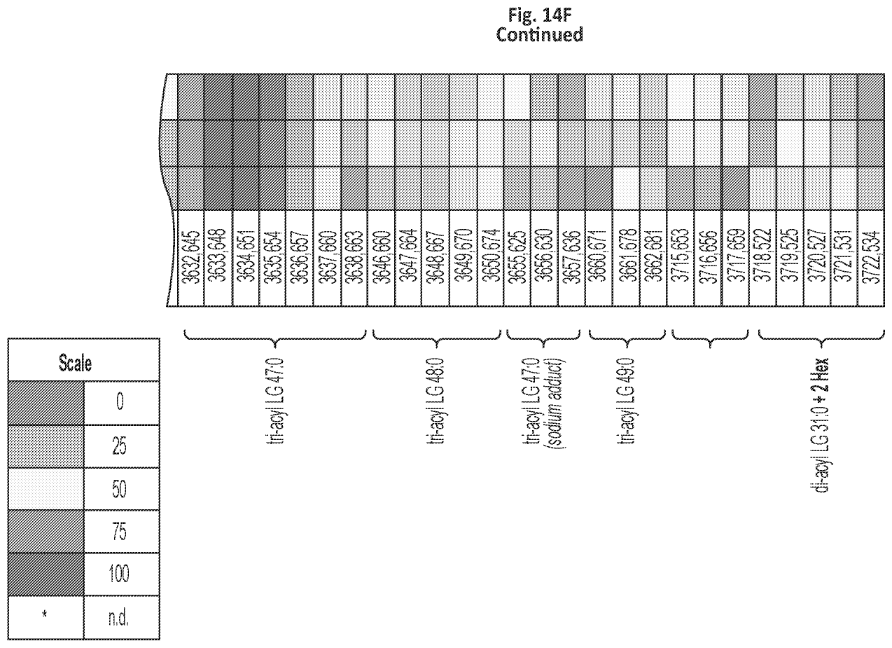

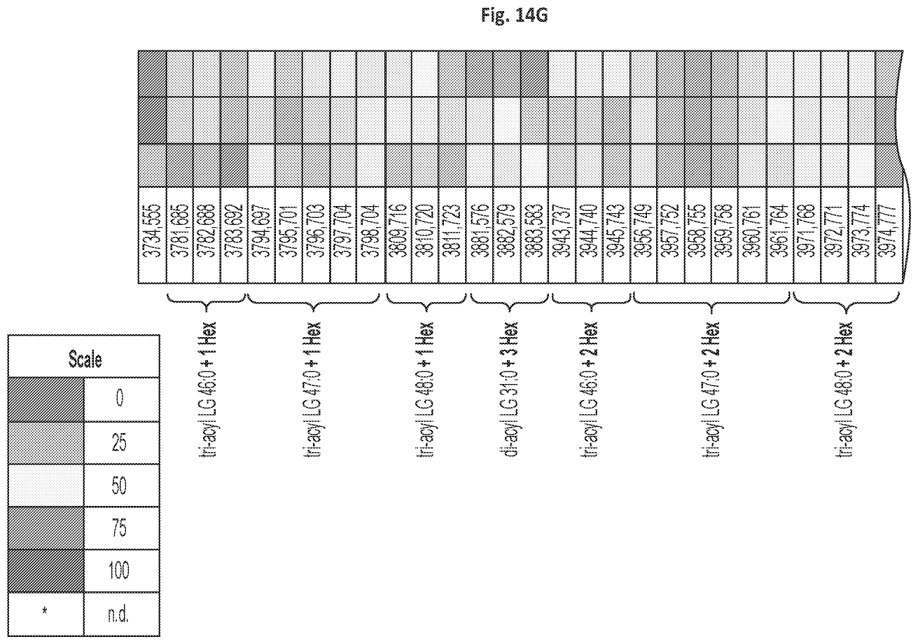

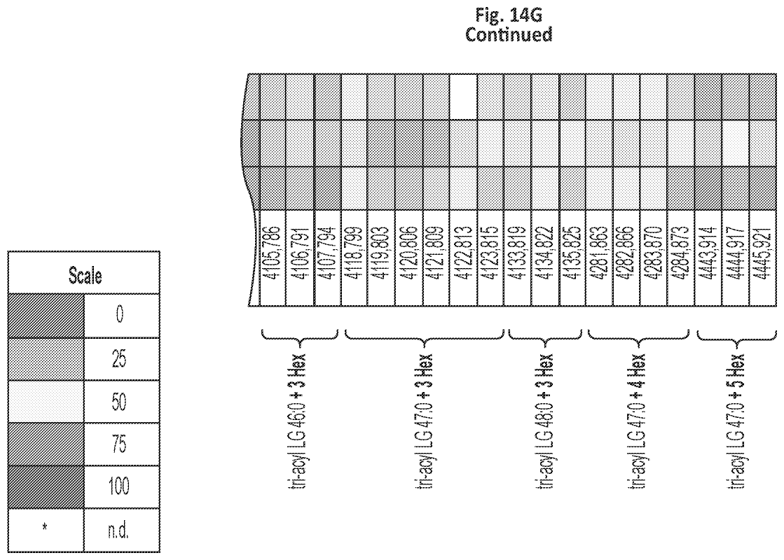

To investigate whether these induced LG-specific Lupus serum antibody responses also recognized antigenic determinants on bacterial strains within the gut of Lupus patients, (i.e., expressed by Lupus-derived RG strains), immunoblots were performed with the serum of the S47 patient, one of the Lupus donors from which RG fecal isolates were obtained at the time of clinical flare (FIGS. 14A-14E). Herein, serum IgG-antibodies were reactive with moieties in extracts of diverse RG strains (FIG. 4A), and after protease digestion of these extracts, there was persistence of IgG-reactive oligobands of the same 25-30 kDa distribution expressed by RG strains, S107-48, S107-86 and S47-18, from the two LN patients, but these antibodies were non-reactive with a RG type strain a healthy donor, ATCC29149 (here termed RG1) and non-reactive with 9 RG strains from IBD patients (RJX1125, RJX1122, RJX1120, RJX1126, RJX1127, RJX1121, RJX1128, RJX1124, RJX1123), and two RG strains from antibiotic-treated infants (RJX1118, RJX1119) all with known genomic sequences (FIGS. 13F-3H) (Hall et al., 2017), were also devoid of these IgG-reactive antigenic oligobands (FIGS. 14A-14C). Such reactivity was also absent with S107-61 strain, but this genome displayed modifications attributable to transposon activity (FIGS. 13F-3H and FIGS. 25A-25C), which may have disrupted genes responsible for LG production (that are currently unidentified).

From evaluations of the relatedness of LG antigens by immunoblot, oligobands were detected in the extracts of the Lupus strains, S107-48, S107-86 and S47-18, that were of the same MW distribution as found in LG purified by hydrophobic interaction column fractionation from the Lupus S47-18 strain (FIGS. 14A-14B). Preincubation of this same Lupus sera with LG purified from RG2 strain resulted in complete inhibition of reactivity with oligobands, in the RG extracts, and the LG purified from the Lupus RG strain, while the lower MW, protease-sensitive band in the RG1 type strain was unaffected (FIGS. 14D-14E).

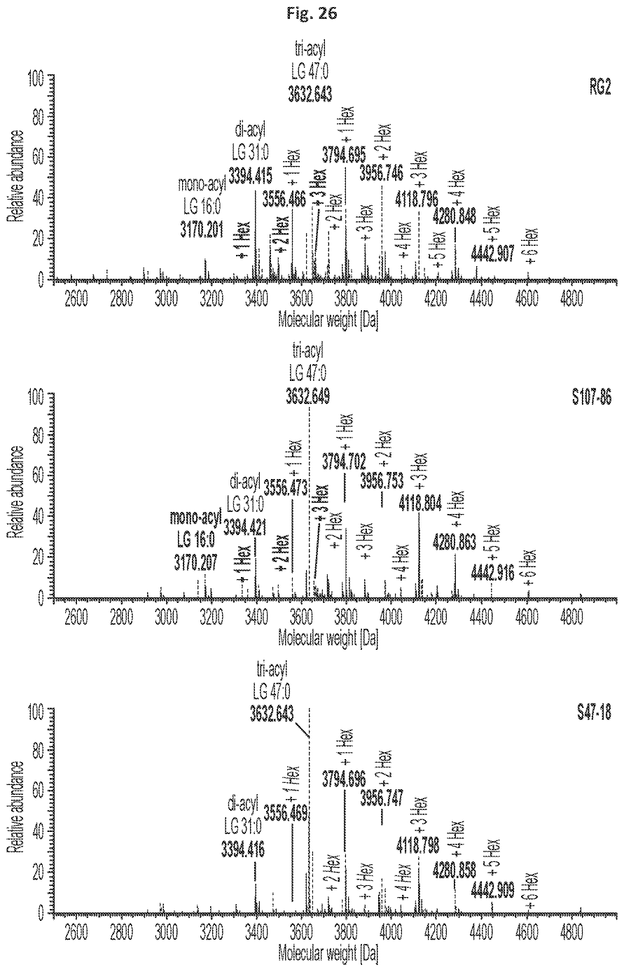

Mass spectrometric (MS) analyses were performed on LGs purified from three isolated RG strains RG2, S107-86, and S47-18 (FIG. 26). The 100 most abundant detected mass signals in the range of 2500-5000 Da are depicted as a heatmap (FIGS. 14F-14G). Ion cluster originating from the same molecular species are grouped by brackets and the LG structural composition is assigned. Using these 100 most abundant peaks, a similarity score was calculated (FIG. 14H), indicating a high similarity between the LG populations of these three strains. The most abundant molecular species in all three strains had an average mono-isotopic mass of 3632.645 Da, which can be assigned to an LG comprised of three fatty acids with acyl chain composition of 47:0, one glycerol, eight hexoses, five N-acetyl-hexosamines, and three hexuronic acids (calc. mono-isotopic mass: 3632.636 Da; tri-acyl LG 47:0). The difference with the major di-acylated LG, with a mass of 3394.415 Da (in RG2; calc. mono-isotopic mass: 3394.407 Da; di-acyl LG 31:0), can be explained by acyl composition of 31:0, just one palmitic acid (16:0) less. As the major mono-acylated species, an LG with 16:0 acyl composition was identified (3170.204 Da; mono-isotopic mass: 3170.193 Da; mono-acyl LG 16:0). In strains RG2 and S107-86, a mono-acyl LG carrying a margaric acid (17:0) was also observed (3184.217 Da in RG2; mono-isotopic mass: 3184.208 Da; mono-acyl LG 17:0). In addition, independent of the acylation status, further variants of the LGs with additional hexoses (up to six detected) were observed, most prominently for the tri-acyl LGs. A detailed summary of the detected LG species in strain RG2 is given in Table 7. Specifically, Table 7 displays calculated and experimentally determined monoisotopic masses of lipoglycan species observed in MS¹ spectra of the LG mixture isolated from strain RG2 shown in FIG. 26 (top panel). Only species with a monoisotopic mass peak abundance>2% are listed. Annotation accuracy of chemical composition to mass measurements are stated as Δppm.

TABLE 7
Detailed summary of the detected LG species in strain RG2

| | Fatty acid | | Calculated | Observed | |
|---|---|---|---|---|---|
| Acylation | sum | | exact mass | monoisotopic | Error |
| status | composition | Glycan composition | [Da] | mass [Da] | [Δppm] |
| mono- | 16:0 | 1 Gro, 8 Hex, 5 | 3170.193 | 3170.201 | 2.5 |
| acyl | | HexNAc, 3 HexU | | | |
| mono- | 16:0 | 1 Gro, 8 Hex, 5 | 3332.246 | 3332.254 | 2.4 |
| acyl | | HexNAc, 3 HexU + 1 | | | |
| | | Hex | | | |
| mono- | 16:0 | 1 Gro, 8 Hex, 5 | 3494.298 | 3494.306 | 2.3 |
| acyl | | HexNAc, 3 HexU + 2 | | | |
| | | Hex | | | |
| mono- | 16:0 | 1 Gro, 8 Hex, 5 | 3656.351 | 3656.358 | 1.9 |
| acyl | | HexNAc, 3 HexU + 3 | | | |
| | | Hex | | | |
| mono- | 17:0 | 1 Gro, 8 Hex, 5 | 3184.208 | 3184.217 | 2.8 |
| acyl | | HexNAc, 3 HexU | | | |
| di-acyl | 30:0 | 1 Gro, 8 Hex, 5 | 3380.391 | 3380.399 | 2.4 |
| | | HexNAc, 3 HexU | | | |
| di-acyl | 31:0 | 1 Gro, 8 Hex, 5 | 3394.407 | 3394.415 | 2.4 |
| | | HexNAc, 3 HexU | | | |
| di-acyl | 31:0 | 1 Gro, 8 Hex, 5 | 3556.460 | 3556.466 | 1.7 |
| | | HexNAc, 3 HexU + 1 | | | |
| | | Hex | | | |
| di-acyl | 31:0 | 1 Gro, 8 Hex, 5 | 3718.512 | 3718.519 | 1.9 |
| | | HexNAc, 3 HexU + 2 | | | |
| | | Hex | | | |
| di-acyl | 31:0 | 1 Gro, 8 Hex, 5 | 3880.565 | 3880.570 | 1.3 |
| | | HexNAc, 3 HexU + 3 | | | |
| | | Hex | | | |
| di-acyl | 31:0 | 1 Gro, 8 Hex, 5 | 4042.618 | 4042.621 | 0.7 |
| | | HexNAc, 3 HexU + 4 | | | |
| | | Hex | | | |
| di-acyl | 32:0 | 1 Gro, 8 Hex, 5 | 3408.422 | 3408.431 | 2.6 |
| | | HexNAc, 3 HexU | | | |
| di-acyl | 32:0 | 1 Gro, 8 Hex, 5 | 3570.475 | 3570.482 | 2.0 |
| | | HexNAc, 3 HexU + 1 | | | |
| | | Hex | | | |
| di-acyl | 32:0 | 1 Gro, 8 Hex, 5 | 3732.528 | 3732.534 | 1.6 |
| | | HexNAc, 3 HexU + 2 | | | |
| | | Hex | | | |
| di-acyl | 32:0 | 1 Gro, 8 Hex, 5 | 3894.581 | 3894.585 | 1.0 |
| | | HexNAc, 3 HexU + 3 | | | |
| | | Hex | | | |
| di-acyl | 33:0 | 1 Gro, 8 Hex, 5 | 3422.438 | 3422.442 | 1.2 |
| | | HexNAc, 3 HexU | | | |
| di-acyl | 33:0 | 1 Gro, 8 Hex, 5 | 3746.544 | 3746.543 | −0.3 |
| | | HexNAc, 3 HexU + 2 | | | |
| | | Hex | | | |
| di-acyl | 33:0 | 1 Gro, 8 Hex, 5 | 3908.597 | 3908.591 | −1.5 |
| | | HexNAc, 3 HexU + 3 | | | |
| | | Hex | | | |
| tri-acyl | 45:0 | 1 Gro, 8 Hex, 5 | 3604.605 | 3604.612 | 1.9 |
| | | HexNAc, 3 HexU | | | |
| tri-acyl | 46:0 | 1 Gro, 8 Hex, 5 | 3618.621 | 3618.627 | 1.7 |
| | | HexNAc, 3 HexU | | | |
| tri-acyl | 46:0 | 1 Gro, 8 Hex, 5 | 3780.674 | 3780.679 | 1.3 |
| | | HexNAc, 3 HexU + 1 | | | |
| | | Hex | | | |
| tri-acyl | 46:0 | 1 Gro, 8 Hex, 5 | 3942.726 | 3942.731 | 1.3 |
| | | HexNAc, 3 HexU + 2 | | | |
| | | Hex | | | |
| tri-acyl | 46:0 | 1 Gro, 8 Hex, 5 | 4104.779 | 4104.782 | 0.7 |
| | | HexNAc, 3 HexU + 3 | | | |
| | | Hex | | | |
| tri-acyl | 47:0 | 1 Gro, 8 Hex, 5 | 3632.636 | 3632.643 | 1.9 |
| | | HexNAc, 3 HexU | | | |
| tri-acyl | 47:0 | 1 Gro, 8 Hex, 5 | 3794.689 | 3794.695 | 1.6 |
| | | HexNAc, 3 HexU + 1 | | | |
| | | Hex | | | |
| tri-acyl | 47:0 | 1 Gro, 8 Hex, 5 | 3956.742 | 3956.746 | 1.0 |
| | | HexNAc, 3 HexU + 2 | | | |
| | | Hex | | | |
| tri-acyl | 47:0 | 1 Gro, 8 Hex, 5 | 4118.795 | 4118.796 | 0.2 |
| | | HexNAc, 3 HexU + 3 | | | |
| | | Hex | | | |
| tri-acyl | 47:0 | 1 Gro, 8 Hex, 5 | 4280.848 | 4280.856 | 1.9 |
| | | HexNAc, 3 HexU + 4 | | | |
| | | Hex | | | |
| tri-acyl | 47:0 | 1 Gro, 8 Hex, 5 | 4442.901 | 4442.907 | 1.4 |
| | | HexNAc, 3 HexU + 5 | | | |
| | | Hex | | | |
| tri-acyl | 48:0 | 1 Gro, 8 Hex, 5 | 3646.652 | 3646.657 | 1.4 |
| | | HexNAc, 3 HexU | | | |
| tri-acyl | 48:0 | 1 Gro, 8 Hex, 5 | 3808.705 | 3808.709 | 1.1 |
| | | HexNAc, 3 HexU + 1 | | | |
| | | Hex | | | |
| tri-acyl | 48:0 | 1 Gro, 8 Hex, 5 | 3970.758 | 3970.760 | 0.5 |
| | | HexNAc, 3 HexU + 2 | | | |
| | | Hex | | | |
| tri-acyl | 48:0 | 1 Gro, 8 Hex, 5 | 4132.811 | 4132.811 | 0.0 |
| | | HexNAc, 3 HexU + 3 | | | |
| | | Hex | | | |
| tri-acyl | 48:0 | 1 Gro, 8 Hex, 5 | 4294.863 | 4294.871 | 1.9 |
| | | HexNAc, 3 HexU + 4 | | | |
| | | Hex | | | |
| tri-acyl | 49:0 | 1 Gro, 8 Hex, 5 | 3660.668 | 3660.669 | 0.3 |
| | | HexNAc, 3 HexU | | | |
| tri-acyl | 49:0 | 1 Gro, 8 Hex, 5 | 3822.721 | 3822.722 | 0.3 |
| | | HexNAc, 3 HexU + 1 | | | |
| | | Hex | | | |
| tri-acyl | 49:0 | 1 Gro, 8 Hex, 5 | 3984.773 | 3984.770 | −0.8 |
| | | HexNAc, 3 HexU + 2 | | | |
| | | Hex | | | |
| tri-acyl | 49:0 | 1 Gro, 8 Hex, 5 | 4146.826 | 4146.820 | −1.4 |
| | | HexNAc, 3 HexU + 3 | | | |
| | | Hex | | | |
| tri-acyl | 50:0 | 1 Gro, 8 Hex, 5 | 3674.683 | 3674.681 | −0.5 |
| | | HexNAc, 3 HexU | | | |

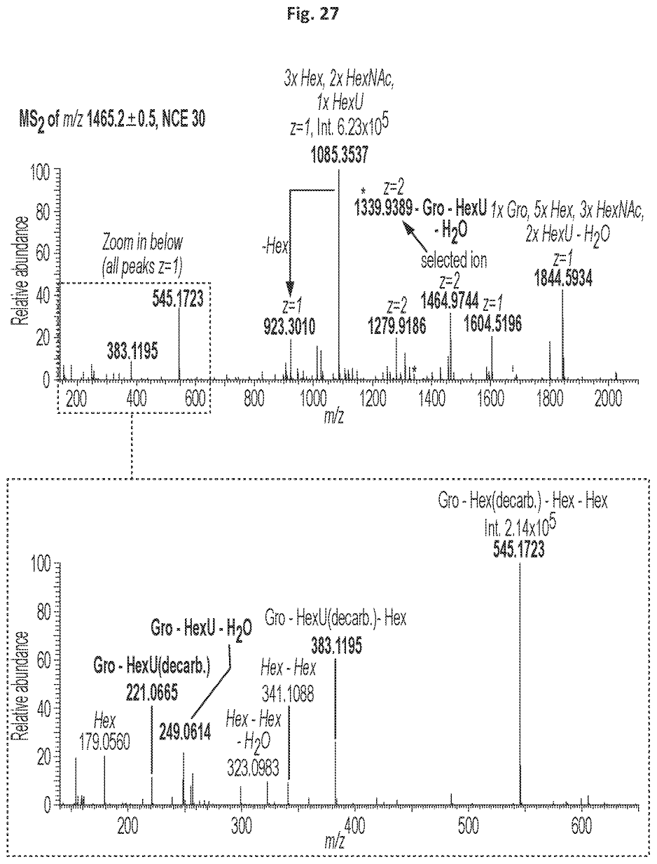

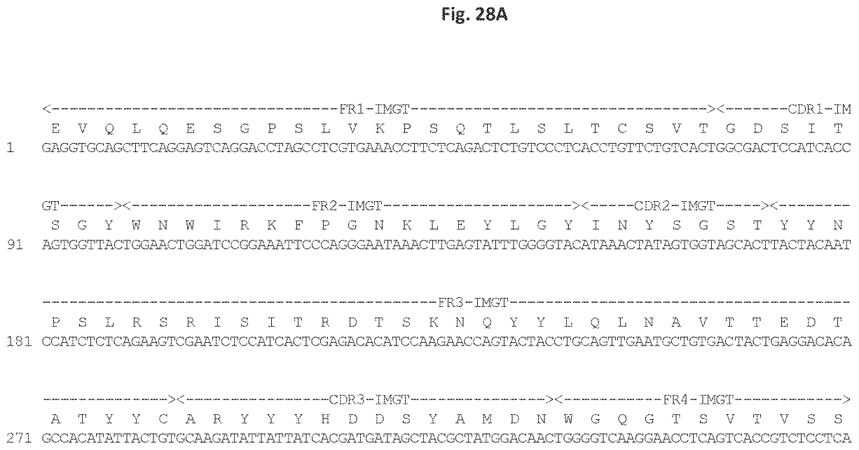

To gain further insight into the composition of the glycolipid anchor structure of these LGs, an earlier reported de-O-acyl LG sample (there named de-O-acyl LG3; (Azzouz et al., 2019)) was used for MS²-experiments. The analysis of the doubly charged ion of the core de-O-acyl LG (calc. mono-isotopic mass: 2931.963 Da) is presented in FIG. 27. The interpretation of fragment ions indicates the presence of a glycerol-hexuronic acid unit that can further consist at least two additional hexoses. Taken together, the mass spectrometric analyses allowed the formulation of a structure model of the major abundant LG species (FIG. 14I) that accounts for all abundant mass spectrometric signals of the analyzed strains.

### Example 15. Post-Immunization Murine Antibodies Recognize Structurally Conserved LGs from Lupus RG Strains

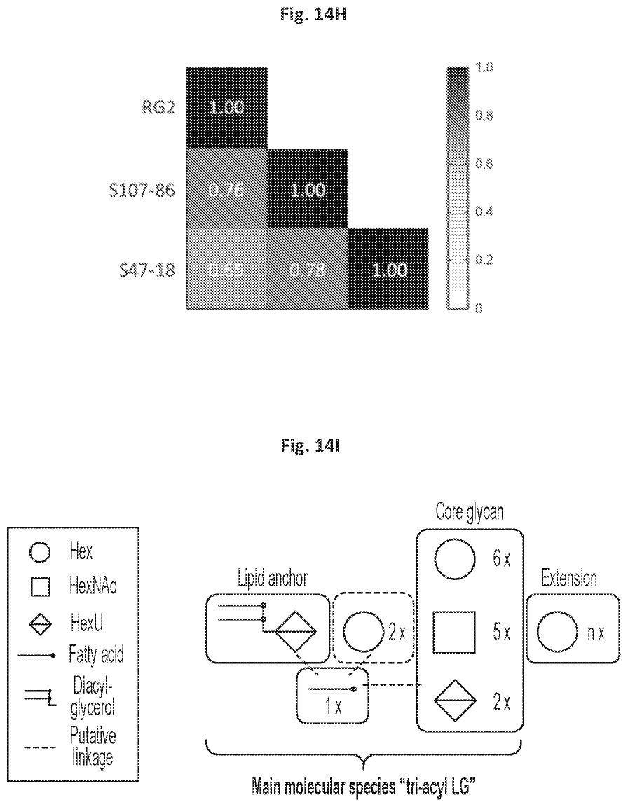

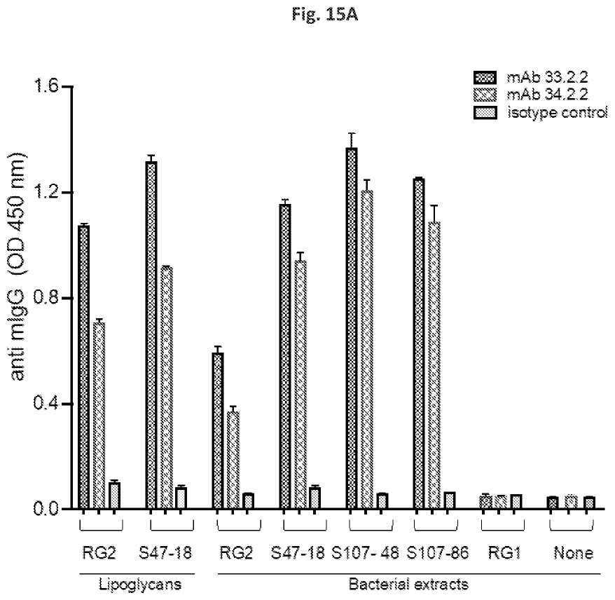

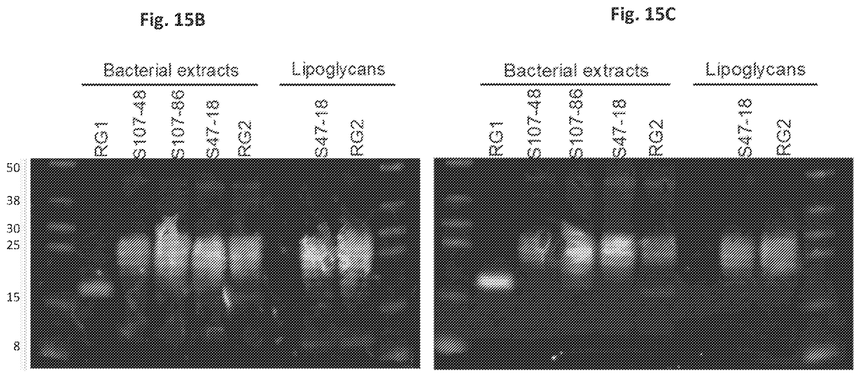

To provide an independent approach to investigate the antigenic diversity expressed by different RG strains, murine monoclonal antibodies were generated by RG bacterial immunization (see below-described methods). By ELISA, both the mAb 33.2.2 and mAb 34.2.2 strongly react with the purified LGs from the S47-18 strain used for the immunization boost and the purified LG from the RG2 strain (FIG. 15A). Both mAbs were reactive with oligobands of the same MW in bacterial extracts from the RG2 strain and Lupus strains, S47-18, S107-48 and S107-86, that were isolated from two different LN patients (FIG. 15A). However, these antibodies were non-reactive with the RG1 extract (FIG. 15A).

The commonality of these recognized non-protein antigens was confirmed based on immune reactivity with these two mAbs. These mAbs recognized oligoband antigen of the same MW in extracts of the Lupus RG strains from two patients, S107-48, S107-86 and S47-18, as well as the index RG2 strain. These mAbs also bound purified LG from both the S47-18 strain and RG2, while there was no reactivity with the extract of RG1, a strain type from a healthy donor (FIG. 15B-15C). Notably, the mAb 33.2.2 is of the IgG2a subclass, while mAb 34.2.2 is of the IgG1 subclass, which suggests these are products of B-cell clones that express independent antibody gene rearrangements that are convergent in encoding for binding of RG LG antigen-specificity. The CDR and variable region sequences for mAb 33.2.2 and mAb 34.2.2 are shown in Table 8. Taken together, these findings documented that RG strains, which colonize different Lupus patients, express structurally related (see, e.g., FIG. 13 and FIG. 27) highly immunogenic, cell wall-associated LGs with conserved cross-reactive antigens, and these LGs are recognized by antibodies that spontaneously arise within human immune systems of many Lupus patients, and murine monoclonal antibodies that were induced by bacterial immunization. By contrast, these LG do not appear to be commonly expressed by RG strains from healthy donors, or by RG strains from IBD patients that in some cases are reported to express forms of capsular polysaccharides (Henke et al., 2021; Henke et al., 2019).

TABLE 8
CDR and variable region sequences for mAb 33.2.2 and mAb 34.2.2

| | HCDR1 | HCDR2 | HCDR3 | VH | | LCDR1 | | LCDR3 | VL | |
|---|---|---|---|---|---|---|---|---|---|---|
| | Amino | Amino | Amino | Amino | | Amino | | Amino | Amino | |
| | acid | acid | acid | acid | DNA | acid | LCDR2 | acid | acid | DNA |
| | sequence | sequence | sequence | sequence | sequence | sequence | Amino | sequence | sequence | sequence |
| mAb | (SEQ ID | (SEQ ID | (SEQ ID | (SEQ ID | (SEQ ID | (SEQ ID | acid | (SEQ ID | (SEQ ID | (SEQ ID |
| # | NO) | NO) | NO) | NO) | NO) | NO) | sequence | NO) | NO) | NO) |
| mAb | 21 | 22 | 3 | 4 | 5 | 24 | KAS | 8 | 9 | 10 |
| 33.2.2 | | | | | | | | | | |
| mAb | 21 | 25 | 12 | 13 | 14 | 24 | KAS | 8 | 15 | 16 |
| 34.2.2 | | | | | | | | | | |

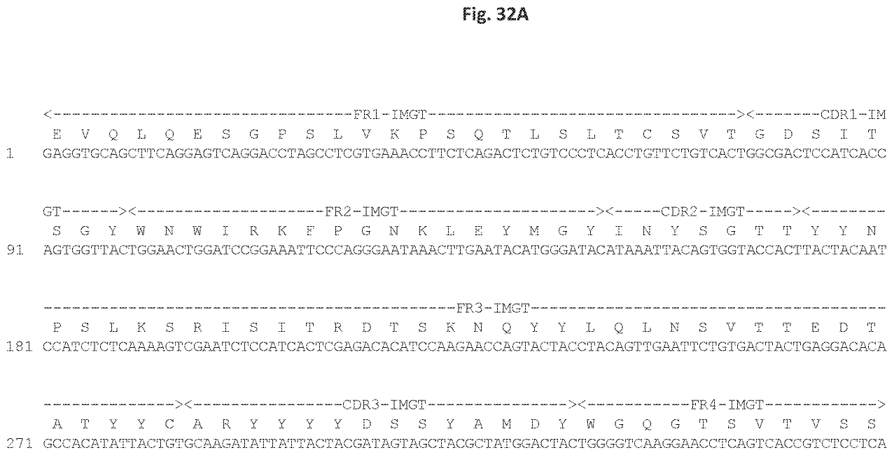

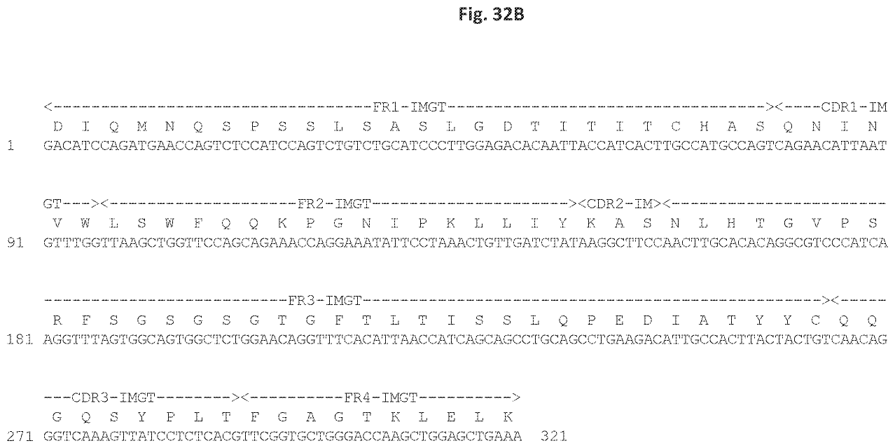

An IMGT definition of the CDRs of mAb 34.2.2 is shown in FIGS. 32A-32B.

### Example 16. High-Titer Antibody Responses Arise Against Structurally Conserved RG Lipoglycans of Lupus Strain Associated Blooms

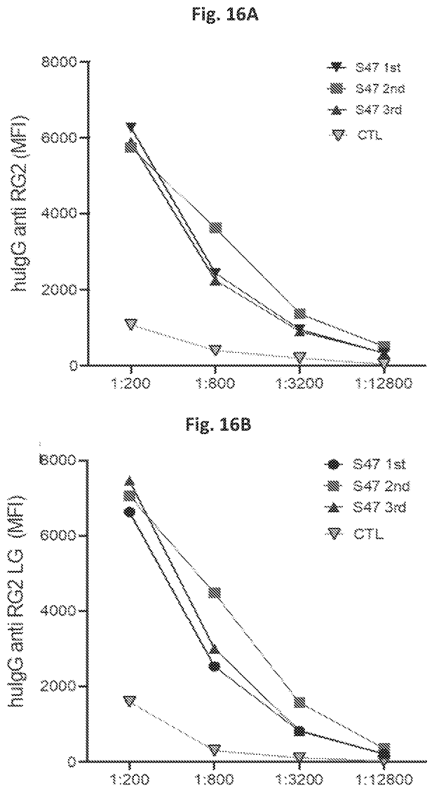

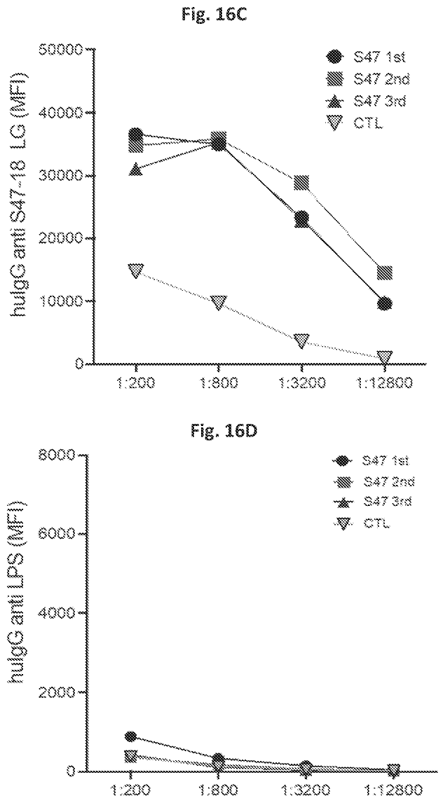

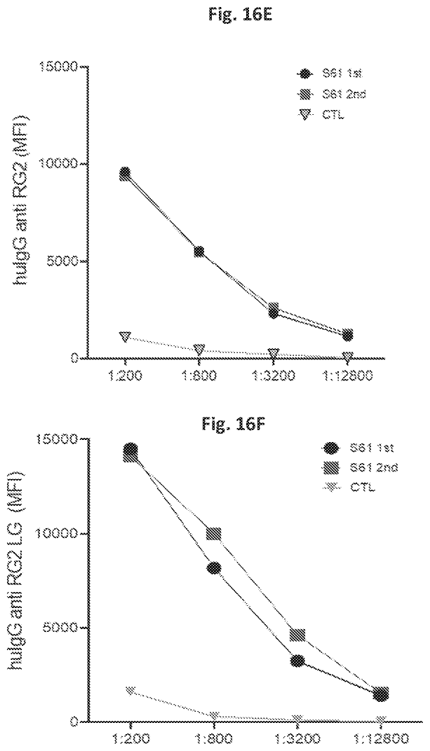

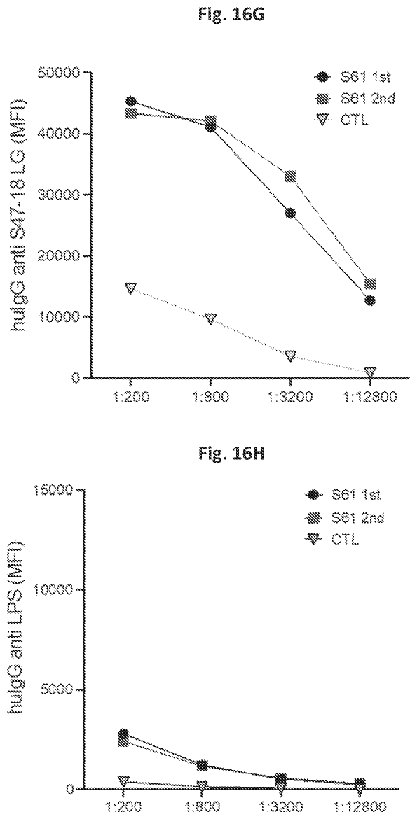

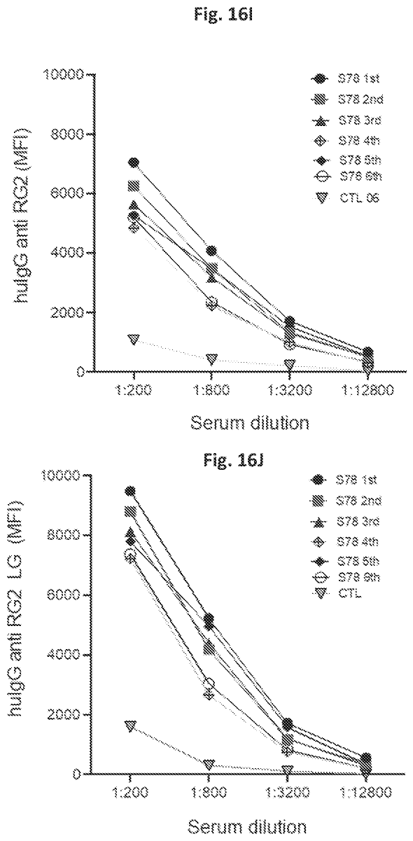

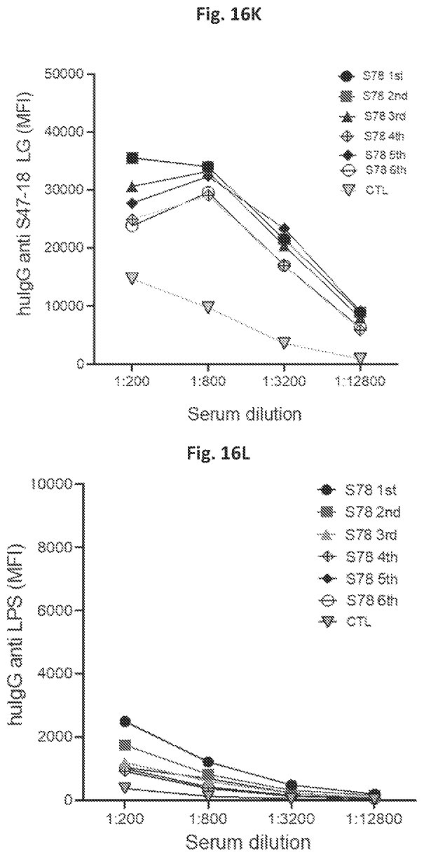

To investigate the Lupus host immune response to the expansion of RG strains overtime, the available longitudinal sera from three Lupus patients was studied (S47, FIGS. 16A-16D; S61, FIGS. 16E-16H; and S78, FIGS. 16I-16L), which included serum samples obtained at the time of clinical LN flare (see FIG. 12C). Serum IgG-reactivities, at multiple dilutions, were assessed for binding interactions with whole bacterial extract of RG2, the index RG strain first shown to contain an immunogenic LG (FIG. 16A, FIG. 16E, and FIG. 16I) (Azzouz et al., 2019), with comparisons of IgG-reactivities with purified LG from this same RG2 strain (i.e., RG2 LG) (Azzouz et al., 2019) (FIG. 16B, FIG. 16F, and FIG. 16J). The near identical reactivity patterns of whole bacterial extracts and purified LG confirm the high immunogenicity of the LG within the RG2 bacterial extract. The same patterns of IgG-reactivities were documented with the structurally-related LG from the S47-18 strain (S47-18 LG) obtained from the S47 LN donor (FIG. 14D and FIG. 14E; FIGS. 5A-5B), although for this LG there were uniformly stronger binding interactions with the IgG in sera from each of the three LN patients (as IgG-binding curves for S47 LG were shifted up and to the right, FIG. 16C, FIG. 16G, and FIG. 16K). By contrast, there was little or no detectable reactivity with the unrelated LPS glycan from a Pseudomonas species (FIG. 16D, FIG. 16H, FIG. 16L) that is not known to bloom within these subjects. Taken together, these data revealed high-titer Lupus host LG-specific serum antibody reactivities, with peak levels in Lupus patients S47 and S61 at the time of disease flare, which was also concordant with an RG bloom in abundance. For patient S78, the antibody titers were relatively invariant, and substantial disease activity was documented at most visits (FIG. 16I, FIG. 16J, and FIG. 16K). Notably, the strongest antibody reactivity of the S47 serum antibody reactivity was with the purified LG from an RG strain isolated from a S47 fecal sample (FIG. 16C), which was obtained at time of disease flare and RG bloom. Parallel highest IgG-reactivity was also found in the sera of the other two Lupus patients (FIG. 16G and FIG. 16K). These findings documented the intertwined nature of Lupus disease flares, which involve immune-complex mediated pathogenetic pathways (Pedersen et al., 2015), and at time with host immune responses to a novel RG Lupus strain-associated LG (FIG. 16).

The longitudinal studies reported herein demonstrated temporal instability within Lupus gut microbiota communities from which blooms of RG blooms arise concordant with Lupus disease flares (FIG. 11 and FIG. 12). The level of variance over time within these communities did not correlate with numbers of samples from a donor, the timespan of sampling, or the features of the disease activity. This variance was neither correlated with maximum SLEDAI disease score during the period of study nor span from lowest to highest SLEDAI score. Furthermore, the greater variance in Lupus patients also did not appear to correlate with medication taken (Tables 1-2 and Tables 5-6). Hence, the inherent instability of the gut microbiota in Lupus patients appears to be a feature inherent to the disease.

Whereas RG was consistently documented only at low stable levels overtime in healthy subjects that was not the case in SLE. By contrast, in a subset of the Lupus patients, representing 4/9 LN patients, the dynamic RG blooms were strikingly concordant with periods of greatly increased disease activity, which in almost every case was manifest as flares involving renal disease. However, in one other Lupus patient (S61) who had no history of renal involvement, RG gut bloom was concurrent with an arthritis flare involving multiple joints. These RG bloom-associated disease flares occurred in White, African American, Asian and Hispanic patients (Table 1), which may suggest that while RG blooms trigger flares of Lupus disease activity, the patterns of organ involvement reflect patient-specific factors—there are likely genetic factors that predispose to renal involvement, akin to differences in murine Lupus strains that are susceptible to, or are protected from, nephritis. Autoantibody profiles may also be contributory, as certain types of IgM-autoantibodies may be protective from renal injury (Gronwall et al., 2012), although antibody levels to RG were not here evaluated.

The RG strains recovered from Lupus patients with blooms produces a novel lipoglycan, with pro-inflammatory properties, and structural features that were conserved based on mass spectrometric analysis, and conserved antigenic determinants recognized by murine monoclonal antibodies. These lipoglycans were highly immunogenic, inducing high-titer systemic IgG responses, which paralleled RG abundance in cross-sectional and longitudinal studies, in particular, in LN with active disease (FIG. 16), and in cohorts across the United States (Azzouz et al., 2019). High-titer IgG antibodies to RG LG were also documented in active LN in a European cohort (Silverman, 2019), suggesting this linkage has no simple geographic restriction.

Emerging evidence further suggests that subclinical disturbances in gut-barrier function found in other conditions (Fasano, 2011) may be common in Lupus patients (Azzouz et al., 2019; Ogunrinde et al., 2019). Indeed, in a murine Lupus model, a gut pathobiont was shown to translocate from the intestine and cause an inflammatory condition (Manfredo Vieira et al., 2018). Furthermore, fecal transplants from mice with Lupus-like glomerulonephritis induced an inflammatory and autoimmune state in otherwise non-autoimmune germ-free C57BL/6 mice (Choi et al., 2020), which clearly demonstrated the pathogenic potential of some gut microbiota communities. It is therefore relevant that murine colonization with RG strains expressing this lipoglycan, but not the control RG1 strain, induced severe gut leakiness, and production of antibodies to the lipoglycan, as well as to native DNA (Deng et al., 2021), a hallmark of Lupus. Within the cross-sectional studies, levels of anti-RG2 antibodies also significantly correlated with serum levels of IL-6, type I interferon, TNF-α, and IL-10 (Azzouz et al., 2019). Taken together, anti-RG responses arise in concert with autoantibody production, which may be a consequence of bacterial factors contributing to the stimulation of autoreactive B cells and disease-specific autoantibody production in predisposed hosts. Indeed, in the serial blood samples from Lupus patients, antibody responses to RG, and specifically for the RG lipoglycan, paralleled the level of RG gut abundance (FIG. 21). In an earlier report, levels of antibodies to RG2 strain (CC55_001C) which was dominated by recognition of LG antigen, inversely correlated with C3 and C4 levels, which was suggestive of an associated immune complex disease (Azzouz et al., 2019). In addition, anti-RG2 levels correlated with IgG anti-dsDNA levels.

RG is a member of the Lachnospiraceae family that plays pleotropic roles in host metabolism and immunity (reviewed in (Vacca et al., 2020)), including bile acid metabolism (Buffie et al., 2015) and the production of the short-chain fatty acids (SCFAs) that aid immune regulation (Arpaia et al., 2013), and RG has otherwise generally been associated with pro-homeostatic communities (Henke et al., 2021). It is therefore important to note that based on whole genome differences, as well as 16S rDNA sequence variations, RG is genetically distinct from other members of this family, and from other Blautia isolates (Sorbara et al., 2020). Importantly, the RG strains in the blooms in Lupus appear to be different than the RG expansions that have also been documented in independent cohorts with IBD (Breban et al., 2017; Hall et al., 2017), including patients with IBD associated spondyloarthritis in whom RG abundance correlated with joint disease activity (Breban et al., 2017). Yet, IBD is a disease of the bowel in which RG expansions may be directly involved in tissue injury inherent to the disease, and examination of 9 RG strains from IBD patients found no detectable lipoglycan (FIGS. 14A-14D). IBD patients do not have prominent associations with autoantibody production or immune complex disease, while susceptibility based on Class I HLA B27 alleles has pointed towards very different mechanistic roles of the gut microbiome with IL-17/23 associated pathology (Dumas et al., 2020). By contrast, while systemic autoantibody production and immune complex disease are integral to the pathogenesis of Lupus, clinically apparent colitis is not a common complication (Koutroubakis et al., 1998).

While key structural and antigenic features of the R. gnavus (RG) lipoglycan (LG) have now been documented to have remarkable conservation between different LN-derived strains (FIG. 14), much remains to be defined in the structural and compositional nature of RG LGs. Under the cultivation conditions used in this study, only the LGs earlier described as B-series (Azzouz et al., 2019) have been observed. Further, in contrast to earlier work in which di-acylated LGs were the predominant species, mainly tri-acylated species were observed. The fact, that the earlier LG preparation of strain RG2, but also all new LG preparations from strains RG2, S107-86, and S47-18, respectively, resulted in the same band pattern in immunoblots with serum IgG-antibodies from LN patients points to the involvement of LGs from the B-series in this interaction. Two of the best investigated LGs from other bacteria are the Corynebacterium glutamicum lipomannan (CgLM) (Mishra et al., 2008; Tatituri et al., 2007) and the Lipomannan (LM) from Mycobacterium tuberculosis [reviewed in (Mishra et al., 2011)]. The LGs observed in R. gnavus strains share some feature similarities to these glycans, but in turn there are also distinct differences. The glycolipid anchor of CgLM-B consists of a diacylglycerol (GroAc2) with a glucuronic acid (GlcA) and a mannose (Man) residue attached, and ManGlcAGroAc2 has been isolated as a major glycolipid from C. glutamicum cells (Tatituri et al., 2007). A very similar anchor structure was identified in the present Examples for the LG of R. gnavus strains. The membrane anchor of M. tuberculosis LM is a mannosylated phosphatidylinositol, which can occur with a third or even a fourth fatty acid attached to the inositol or the mannose residue, respectively [reviewed in (Mishra et al., 2011)]. This feature in the context of a third fatty acid was observed for the LG of R. gnavus strains as well. However, unlike the LG of R. gnavus, neither the presence of further hexuronic acids nor of N-acetyl hexosamines in the core glycan has been described in the other lipoglycans, in which the glycan core comprises only mannose residues, while the LG in RG has only hexose moieties. Therefore, the lipoglycan observed in the R. gnavus strains described herein can be considered as unique.

Taken together, the findings disclosed herein document the conservation of an LG with conserved structural and antigenic determinants in the RG strains isolated from blooms in active LN patients. The responsible genes and molecular enzymatic pathways responsible for assembly still need to be defined. Their identification would be of great value to be able to interfere genetically in the LG biosynthesis, e.g., for reduction of the glycan complexity by preventing the hexose extensions, potentially enabling a full structure elucidation of the core glycan.

Findings disclosed herein also support the notion that RG expansions can directly contribute to Lupus disease flares. This does not exclude the potential involvement of other pathobionts, and/or contractions of “protective” species such as Faecalibacterium prausnitizii that also occur during active Lupus disease (Azzouz et al., 2019). Overall, the present findings appear to reflect the same altered gut community patterns documented in Chimpanzees after Simian Immunodeficiency Virus (SIV) infection, in which gut microbiota community instability arises and transient spikes/blooms also emerge (Moeller et al., 2013). This report suggests that this gut community (dys-) regulation is likely influenced by CD4⁺ T-cells that are selectively deleted by SIV (Katsuyama et al., 2018). There may be important parallels as SLE patients are known to have generalized defects in T-cell regulation (Suarez-Fueyo et al., 2016). Moreover, in health CD4⁺ T-cells play central roles of in the local maintenance of the gut barrier (Marks and Craft, 2009), although the implications of human Lupus associated T-cell dysregulation for gut bacterial expansions and altered intestinal permeability have not been examined.

In conclusion, an overarching question of whether in healthy individuals RG of some strains provides benefits to the host, while in susceptible Lupus patients, RG blooms of some but not other strains can stoke systemic inflammation and tissue injury, must now be considered. The pathogenic potential of an RG strain may be influenced by the production of glycans such as the tolerogenic capsular polysaccharide isolated from an RG strain from a healthy donor (Henke et al., 2021) while other RG strains instead produce a highly immunogenic pro-inflammatory cell wall lipoglycan (Azzouz et al., 2019). Therefore, as multiple RG strains have been isolated from a single donor (Sorbara et al., 2020), disease flares could also result from intra-community shifts in the representation of individual RG strains that differ based on their associated sets of genes that may determine their pathogenic potential.

Below are the methods used in Examples 8-15 described above.

Studies were performed under the supervision of the NYU Institutional Human Subjects Committee, and all subjects provided written informed consent. Exclusion and inclusion criteria for patients and healthy controls have previously been described (Azzouz et al., 2019). These specific criteria, other aspects of the clinical studies, and the collection and characterization of the gut microbiota is described in the supplement (Table 1).

Isolation, characterization, genome sequence determination of RG colonies. To recover R. gnavus colonies, fecal samples were streaked onto TSBA or BHI plates and grown under anaerobic conditions. Individual colonies, identified based on morphology and growth characteristics of the strains ATCC29149 (termed RG1) and CC55_001C (termed RG2), were sub-streaked and subcultured. From each colony genomic DNA was recovered with the power soil (Qiagen™) kit, according to the manufacturer's instructions, quantified on a Nanodrop 1000 (Thermo Scientific). To assess total 16S rDNA gene, PCR assays were performed with the T100 thermocycler (Bio-Rad™) using the primers (Png et al., 2010):

UniF340
(SEQ ID NO: 17)
(5′-ACTCCTACGGGAGGCAGCAGT-3′)
UniR514
(SEQ ID NO: 18)
(5′-ATTACCGCGGCTGCTGGC-3′)

94° C. for 3 minutes, followed by 35 cycles of 94° C. for 45 seconds, 50° C. for 1 min; and followed by extension at 72° C. for 10 minutes and 4° C. hold.

The RG species-specific 16S rDNA was determined with the previously reported oligonucleotide primers (Png et al., 2010):

Fwd
(SEQ ID NO: 19)
5′-GGACTGCATTTGGAACTGTCAG-3′
Rev
(SEQ ID NO: 20)
5′-AACGTCAGTCATCGTCCAGAAAG-3′

The following cycles were used: an initial 94° C. for 3 minutes, followed by 35 cycles of 94° C. for 45 seconds, 58° C. for 1 min; and followed by extension at 72° C. for 10 minutes and 4° C. hold.

Colonies of interest were named based on the Lupus patients that provided the fecal sample of origin, S47 and S107, which were named according to the donor source. DNA from each isolate was then subjected to whole genome sequencing, using both NextSeq 550 Illumina and PacBio technologies. Alignments with the genomes of RG1 and RG2 strains documented that the assignment to the RG species with genomes that affirmed that independent non-identical RG strains (FIGS. 25A-25C).

Bacterial whole-genome sequence analysis. Raw short-read sequence reads were preprocessed using fastp (Chen et al., 2018) version 0.22 using default settings. Preprocessed reads were screened for within-species contamination using ConFindr (Low et al., 2019) as well as cross-species contamination using MetaPhlAn (Beghini et al., 2021). Preprocessed short reads were assembled using Unicycler (Wick et al., 2017) using its conservative mode.

Processing and assembly of PacBio long reads was performed using the PacBio SMRT Link software suite. Assemblies were further assessed by BLAST search (Altschul et al., 1990) against the NCBI nucleotide database. Contigs matching Cutibacterium acnes, a common human skin bacterium, were removed from long-read assemblies, and it was verified that the remaining contigs mapped to corresponding uncontaminated short-read assemblies Genome assemblies were annotated using Prokka (Seemann, 2014). Raw sequence data and assemblies have been deposited to NCBI repositories under BioProject PRJNA821229.

The BEDTools software suite (Quinlan and Hall, 2010) was used to divide genome assemblies into 1 kbp windows and to analyze GC content per window. Each window was compared to RG1 (NCBI accession GCF_009831375.1) and RG2 (GCF_000507805.1) using BLAST (Altschul et al., 1990), keeping only alignments with E values below 10-20. Visualizations of each genome assembly were produced using the circlize R package (Gu et al., 2014).

All genome assemblies linked to the species [Ruminococcus] gnavus (taxonomy ID 33038) in NCBI RefSeq (accessed Mar. 10, 2022) were downloaded for comparison to the newly generated assemblies. One of these (accession number GCF_020538135.1) was found to be highly divergent from the others (with average nucleotide identity<90%) and was excluded from further comparisons. Assessment of gene content across assemblies was performed using Orthofinder (Emms and Kelly, 2019). The resulting presence/absence matrix of orthogroups was used to generate pairwise Jaccard dissimilarities between isolates using the vegan R package (Oksanen, 2020).

Lipoglycan purification and mass spectrometric analysis. RG strains were individually expanded in chopped meat media to stationary phase, pelleted, and LGs were extracted as described earlier (Azzouz et al., 2019). Mass spectrometric analyses of LG preparations were performed on a Q Exactive Plus (ThermoFisher Scientific, Bremen, Germany) using a Triversa Nanomate (Advion, Ithaca, NY) as nano-ESI source. LG extracts were initially dissolved in a concentration of 1 μg μl⁻¹ in water and 10 μl of this solution were mixed with 150 μl of water/propan-2-ol/7 M triethylamine/acetic acid (50:50:0.06:0.02, [v/v/v/v]). Mass spectra were recorded for 0.50 min in the negative mode in an m/z-range of 400-2000 or 500-3000 applying a spray voltage of −1.1 kV. Depicted MS spectra were charge deconvoluted (Xtract module of Xcalibur 3.1 software; ThermoFisher Scientific, Bremen, Germany) and all provided values refer to mono-isotopic masses of neutral molecules. Single scan *.mzmL files were generated with MSconvert (Chambers et al., 2012) and used as import for LipidXplorer 1.2.8 (Herzog et al., 2011) to compute an aligned data set. From this data set, the top hundred intense peaks were used to prepare the heatmap shown in FIGS. 14F-14G, and to compute the similarity score depicted in FIG. 14H based on Spearman rank correlation. For MS2 experiments aiming to analyze the glycolipid linker composition, the de-O-acyl LG3 preparation described earlier (Azzouz et al., 2019) was used. Doubly charged ions of interest were selected and spectra were recorded in the negative ion mode at different normalized collision energies (NCE).

Immunoblotting. Electrophoretic separation used Bis-Tris mini gels (Novex, Thermo Fisher) with bacterial extracts loaded at the same concentration, then transferred to membranes, which were incubated with sera diluted at 1:100, and incubated overnight at 4° C. For detection, anti-human IgG biotin conjugated (Jackson ImmunoResearch Labs, USA) was added and developed by IRDye® 800CW Streptavidin (LI-COR®).

Generation of LG-specific murine monoclonal antibodies. A commercial vendor (Envigo Bioproducts Inc., Indianapolis) immunized 10 BALB/c mice with extract of the RG2 strain emulsified in complete Freund's adjuvant and later boosted with lipoglycan purified from the Lupus S47-18 strain, which was emulsified in incomplete Freund's adjuvant, were purified from an RG strain by a method that included fractionation by hydrophobic interaction chromatography, as previously described (Azzouz et al., 2019). The spleen from the mouse with the strongest post-immunization was fused with Ig-deficient NS-1 myeloma cells. The spent supernatants subclones were evaluated for IgG-reactivity, which demonstrated highly correlated reactivity with whole extracts of the immunizing RG strain and purified RG lipoglycan, with the subcloned hybridoma cell lines, referred to as mAb 33.2.2 and mAb 34.2.2 herein.

Direct binding ELISA. To detect the reactivity of the murine monoclonal antibodies (mAb) 33.2.2 and mAb 34.2.2 with the different RG strains, the ELISA plates were coated with the bacterial extracts from RG2, S47-18, S107-48, S107-86, RG1 as well as with the purified lipoglycan from the strains RG2 and S47-18. Next, the murine monoclonal antibodies were added at two concentrations (at 100 ng/ml and 25 ng/ml) in duplicate, incubated for 2 h at RT. Binding was detected with goat anti mouse IgG HRP conjugated at 1:10,000 (Jackson ImmunoResearch), then TMB substrate was added to develop the plate.

Multiplex bead-based immunoassay. The assays were performed as described previously (Azzouz et al., 2019). Briefly, serum samples from patients and healthy controls underwent to 4-fold serial dilutions starting at 1:200 to 1:12,800 against a panel of antigens including the bacterial extracts from Lupus strains RG2, S47-18, RG1 as well as the purified lipoglycan from the strains RG2 and S47-18, then detecting using goat anti-human, PE conjugated (eBioscience).

Statistical analysis. Data are presented as mean+SD. Student unpaired t test with Welch's correction was used in 2-group comparisons of normally distributed data, whereas the Mann-Whitney nonparametric test was used when the normality assumption was not met. Fisher's exact test was performed to evaluate bivariate associations between categorical variables, or as described. To test for correlations between two variables Spearman test was used. p-values were considered significant at <0.05 for two-tailed tests. Prism software Version 9 (GraphPad) was used for all analyses.

### Example 17. Generation of Chimeric Antibody that Specifically Binds the R. Gnavus Lipoglycan

Towards the goal of generating practical protein-based and/or antibody agents that specifically bind the R. gnavus lipoglycan, by itself or when attached to the R. gnavus bacteria which produces it, a chimeric antibody made with murine antibody variable region genes, that were fused to human IgG2 constant region genes, was generated and high level of binding reactivity was demonstrated.

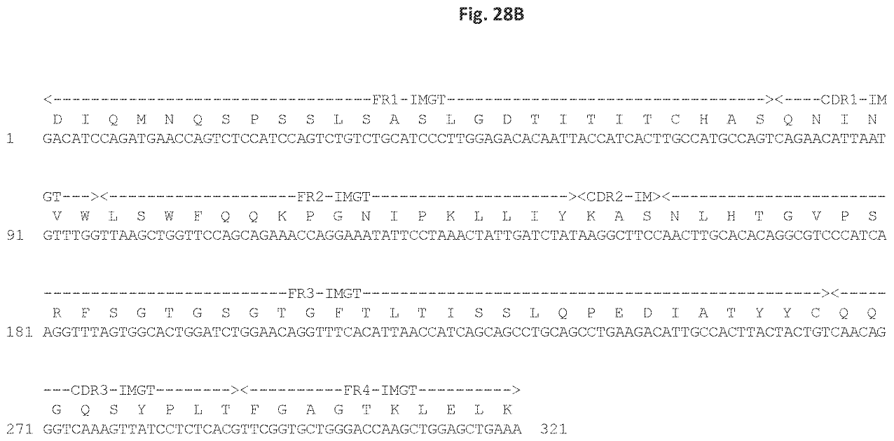

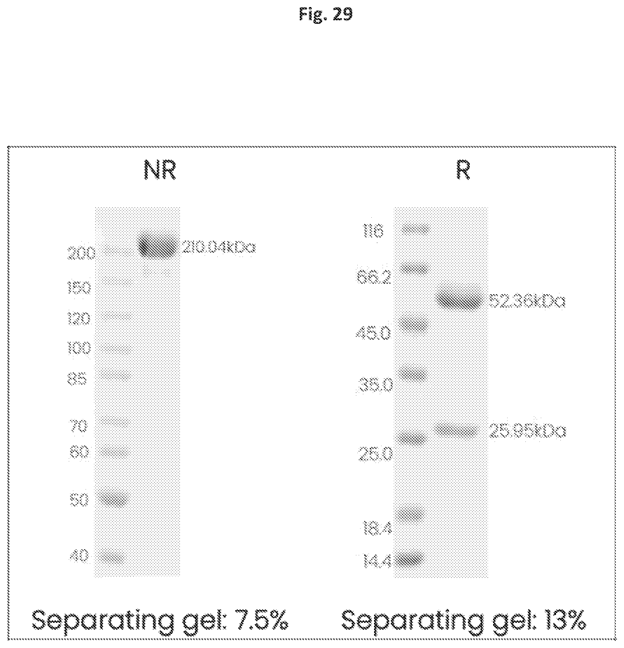

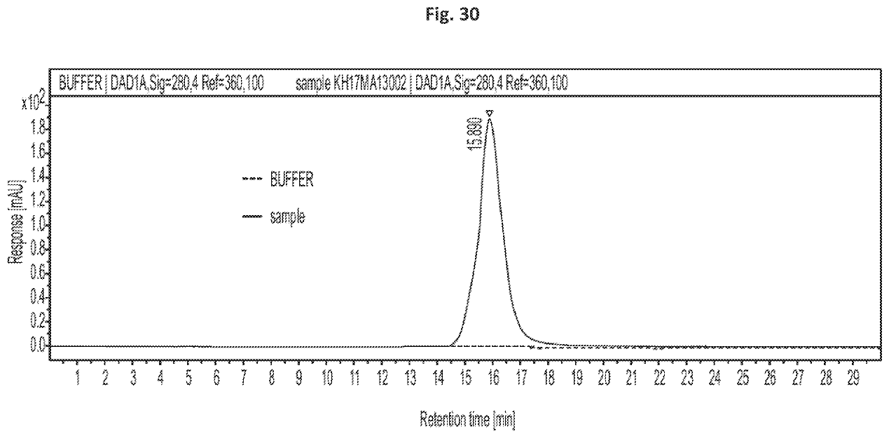

The generation of this chimeric antibody was performed in the following steps:
1) Gene synthesis. Synthetic DNA sequences were generated in which the VH region (FIG. 28A) of the parental 33.2.2 B cell hybridoma cell line was placed upstream (i.e., 5′) to the gene encoding the human gamma 2 subclass constant region. In parallel, the light chain variable region (FIG. 28B) for this cell line was placed upstream of the human kappa constant region gene.
2) Vector construction. These target genes were amplified by polymerase chain reaction, with the use of oligonucleotide primers that facilitated cloning into a compatible plasmid vector.
3) Validation of production of the chimeric antibody expressed in HEK293 cells. For transient transfection, the plasmids were mixed with transfection reagents at an optimal ratio and then added into the culture of HEK293 cells, which were grown in a serum-free medium and maintained in Erlenmeyer Flasks on an orbital shaker or the bioreactor by a suitable stirring speed at 37° C. for 6 days.
4) Purification and Analysis: a) cell culture broth from the growth of the transfected cell line was centrifuged to remove unwanted cells and debris; b) cell culture supernatant was then loaded onto a protein A affinity purification column at an appropriate flow rate, then IgG was eluted with a mild acid buffer with rapid neutralization and dialysis into a physiologic pH buffer; c) the purified protein was analyzed by SDS-PAGE (FIG. 29) and SEC-HPLC (FIG. 30).

### Characterization of the Binding Activity for the Lipoglycan Antigen.

To characterize the capacity of the chimeric antibody to bind the antigen used for initial generation of the parental B cell hybridoma cell line, a validated immunoassay was used, with purified lipoglycan, designated LG3, with a method using the Magpix instrument (Luminex) as previously described (Azzouz et al. Ann Rheum Dis 2019 July; 78(7):947-956.).

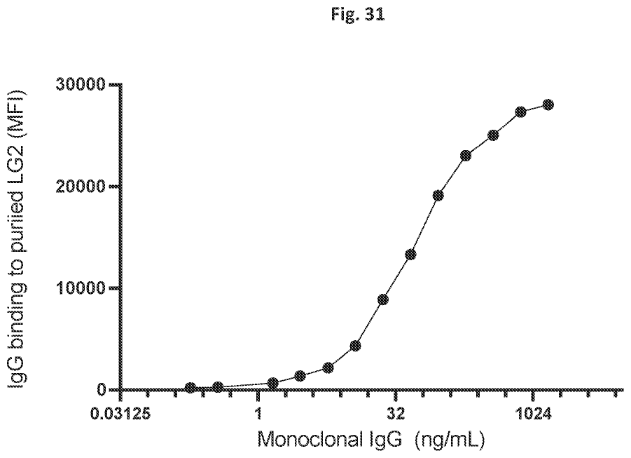

Binding activity was detected with a signal above background for the assay with a IgG concentration of below 400 μg/ml, with activity also documented that was below saturation of the assay using 1500 ng/ml of the chimeric antibody (FIG. 31). Hence, a functional recombinant antibody was produced, purified, and demonstrated to retain high level binding activity for the lipoglycan produced by a strain of R. gnavus.

### REFERENCES

Aliyu, S. H., Marriott, R. K., Curran, M. D., Parmar, S., Bentley, N., Brown, N. M., Brazier, J. S., and Ludlam, H. (2004). Real-time PCR investigation into the importance of Fusobacterium necrophorum as a cause of acute pharyngitis in general practice. J Med Microbiol 53, 1029-1035. 10.1099/jmm.0.45648-0.
Almaani, S., Meara, A., and Rovin, B. H. (2017). Update on Lupus Nephritis. C/in J Am Soc Nephrol 12, 825-835. 10.2215/CJN.05780616.
Altschul, S. F., Gish, W., Miller, W., Myers, E. W., and Lipman, D. J. (1990). Basic local alignment search tool. J Mol Biol 215, 403-410. 10.1016/S0022-2836(05)80360-2.
Arpaia, N., Campbell, C., Fan, X., Dikiy, S., van der Veeken, J., deRoos, P., et al. (2013). Metabolites produced by commensal bacteria promote peripheral regulatory T-cell generation. Nature 504(7480), 451-455. doi: 10.1038/nature12726.
Azzouz, D., Omarbekova, A., Heguy, A., Schwudke, D., Gisch, N., Rovin, B. H., et al. (2019). Lupus nephritis is linked to disease-activity associated expansions and immunity to a gut commensal. Ann Rheum Dis 78(7), 947-956. doi: 10.1136/annrheumdis-2018-214856.
Barr, S. G., Zonana-Nacach, A., Magder, L. S., and Petri, M. (1999). Patterns of disease activity in systemic lupus erythematosus. Arthritis Rheum 42, 2682-2688. 10.1002/1529-0131(199912)42:12<2682::AID-ANR26>3.0. CO; 2-6.
Beghini, F., McIver, L. J., Blanco-Miguez, A., Dubois, L., Asnicar, F., Maharjan, S., Mailyan, A., Manghi, P., Scholz, M., Thomas, A. M., et al. (2021). Integrating taxonomic, functional, and strain-level profiling of diverse microbial communities with bioBakery 3. Elife 10. 10.7554/eLife.65088.
Bell, A., Brunt, J., Crost, E., Vaux, L., Nepravishta, R., Owen, C. D., et al. (2019). Elucidation of a sialic acid metabolism pathway in mucus-foraging Ruminococcus gnavus unravels mechanisms of bacterial adaptation to the gut. Nat Microbiol 4(12), 2393-2404. doi: 10.1038/s41564-019-0590-7.
Bhattarai, Y., and Kashyap, P. C. (2016). Germ-Free Mice Model for Studying Host-Microbial Interactions. Methods Mol Biol 1438, 123-135. doi: 10.1007/978-1-4939-3661-8_8.
Breban, M., Tap, J., Leboime, A., Said-Nahal, R., Langella, P., Chiocchia, G., Furet, J. P., and Sokol, H. (2017). Faecal microbiota study reveals specific dysbiosis in spondyloarthritis. Ann Rheum Dis 76, 1614-1622. 10.1136/annrheumdis-2016-211064.
Brook, I. (1996). Veillonella infections in children. J Clin Microbiol 34, 1283-1285. 10.1128/jcm.34.5.1283-1285.1996.
Buffie, C. G., Bucci, V., Stein, R. R., McKenney, P. T., Ling, L., Gobourne, A., et al. (2015). Precision microbiome reconstitution restores bile acid mediated resistance to Clostridium difficile. Nature 517(7533), 205-208. doi: 10.1038/nature13828.
Chambers, M. C., Maclean, B., Burke, R., Amodei, D., Ruderman, D. L., Neumann, S., Gatto, L., Fischer, B., Pratt, B., Egertson, J., et al. (2012). A cross-platform toolkit for mass spectrometry and proteomics. Nat Biotechnol 30, 918-920. 10.1038/nbt.2377.
Chen, B. D., Jia, X. M., Xu, J. Y., Zhao, L. D., Ji, J. Y., Wu, B. X., et al. (2020). An Autoimmunogenic and Proinflammatory Profile Defined by the Gut Microbiota of Patients With Untreated Systemic Lupus Erythematosus. Arthritis Rheumatol. doi: 10.1002/art.41511.
Chen, S., Zhou, Y., Chen, Y., and Gu, J. (2018). fastp: an ultra-fast all-in-one FASTQ preprocessor. Bioinformatics 34, i884-i890. 10.1093/bioinformatics/bty560.
Choi, S. C., Brown, J., Gong, M., Ge, Y., Zadeh, M., Li, W., Croker, B. P., Michailidis, G., Garrett, T. J., Mohamadzadeh, M., and Morel, L. (2020). Gut microbiota dysbiosis and altered tryptophan catabolism contribute to autoimmunity in lupus-susceptible mice. Sci Transl Med 12. 10.1126/scitranslmed.aax2220.
Deng, J., Azzouz, D. F., Ferstler, N., and Silverman, G. J. (2021). Sex-dependent Lupus Ruminococcus blautia gnavus strain induction of zonulin-mediated intestinal permeability and autoimmunity. bioRxiv 07.06.451365. 10.1101/2021.07.06.451365.
Donaldson, G. P., Lee, S. M., and Mazmanian, S. K. (2016). Gut biogeography of the bacterial microbiota. Nat Rev Microbiol 14(1), 20-32. doi: 10.1038/nrmicro3552.
Dumas, E., Venken, K., Rosenbaum, J. T., and Elewaut, D. (2020). Intestinal Microbiota, HLA-B27, and Spondyloarthritis: Dangerous Liaisons. Rheum Dis Clin North Am 46, 213-224. 10.1016/j.rdc.2020.01.007.
Emms, D. M., and Kelly, S. (2019). OrthoFinder: phylogenetic orthology inference for comparative genomics. Genome Biol 20, 238. 10.1186/s13059-019-1832-y.
Fasano, A. (2012). Zonulin, regulation of tight junctions, and autoimmune diseases. Ann N Y Acad Sci 1258, 25-33. doi: 10.1111/j.1749-6632.2012.06538.x.
Gronwall, C., Akhter, E., Oh, C., Burlingame, R. W., Petri, M., and Silverman, G. J. (2012). IgM autoantibodies to distinct apoptosis-associated antigens correlate with protection from cardiovascular events and renal disease in patients with SLE. Clin Immunol 142, 390-398. 10.1016/j.clim.2012.01.002.
Gopalakrishnan, S., Durai, M., Kitchens, K., Tamiz, A. P., Somerville, R., Ginski, M., et al. (2012). Larazotide acetate regulates epithelial tight junctions in vitro and in vivo. Peptides 35(1), 86-94. doi: 10.1016/j.peptides.2012.02.015.
Gu, Z., Gu, L., Eils, R., Schlesner, M., and Brors, B. (2014). circlize Implements and enhances circular visualization in R. Bioinformatics 30, 2811-2812. 10.1093/bioinformatics/btu393.
Hall, A. B., Yassour, M., Sauk, J., Garner, A., Jiang, X., Arthur, T., Lagoudas, G. K., Vatanen, T., Fornelos, N., Wilson, R., et al. (2017). A novel Ruminococcus gnavus clade enriched in inflammatory bowel disease patients. Genome Med 9, 103. 10.1186/s13073-017-0490-5.
Hamidi, B., Wallace, K., Vasu, C., and Alekseyenko, A. V. (2019). W(*)d-test: robust distance-based multivariate analysis of variance. Microbiome 7, 51. 10.1186/s40168-019-0659-9.
Henke, M. T., Brown, E. M., Cassilly, C. D., Vlamakis, H., Xavier, R. J., and Clardy, J. (2021). Capsular polysaccharide correlates with immune response to the human gut microbe Ruminococcus gnavus. Proc Natl Acad Sci USA 118(20). doi: 10.1073/pnas.2007595118.
Henke, M. T., Brown, E. M., Cassilly, C. D., Vlamakis, H., Xavier, R. J., and Clardy, J. (2021). Capsular polysaccharide correlates with immune response to the human gut microbe Ruminococcus gnavus. Proc Natl Acad Sci USA 118. 10.1073/pnas.2007595118.
Henke, M. T., Kenny, D. J., Cassilly, C. D., Vlamakis, H., Xavier, R. J., and Clardy, J. (2019). Ruminococcus gnavus, a member of the human gut microbiome associated with Crohn's disease, produces an inflammatory polysaccharide. Proceedings of the National Academy of Sciences of the United States of America 116, 12672-12677. 10.1073/pnas.1904099116.
Herzog, R., Schwudke, D., Schuhmann, K., Sampaio, J. L., Bornstein, S. R., Schroeder, M., and Shevchenko, A. (2011). A novel informatics concept for high-throughput shotgun lipidomics based on the molecular fragmentation query language. Genome Biol 12, R8. 10.1186/gb-2011-12-1-r8.
Hochberg, M. C. (1997). Updating the American College of Rheumatology revised criteria for the classification of systemic lupus erythematosus. Arthritis Rheum 40, 1725. 10.1002/1529-0131(199709)40:9&lt; 1725::AID-ART29&gt; 3.0. CO; 2-Y.
Hoffmann, T. W., Pham, H. P., Bridonneau, C., Aubry, C., Lamas, B., Martin-Gallausiaux, C., et al. (2015). Microorganisms linked to inflammatory bowel disease-associated dysbiosis differentially impact host physiology in gnotobiotic mice. ISME J. doi: 10.1038/ismej.2015.127.
Jackson, M. A., Verdi, S., Maxan, M. E., Shin, C. M., Zierer, J., Bowyer, R. C. E., Martin, T., Williams, F. M. K., Menni, C., Bell, J. T., et al. (2018). Gut microbiota associations with common diseases and prescription medications in a population-based cohort. Nat Commun 9, 2655. 10.1038/s41467-018-05184-7.
Katsuyama, T., Tsokos, G. C., and Moulton, V. R. (2018). Aberrant T Cell Signaling and Subsets in Systemic Lupus Erythematosus. Front Immunol 9, 1088. 10.3389/fimmu.2018.01088.
Kim, M., Merrill, J., Kalunian, K., Hahn, B., Roach, A., Izmirly, P., and Lupus Foundation of America Collective Data Analysis Initiative, G. (2017). Brief Report: Longitudinal Patterns of Response to Standard of Care Therapy for Systemic Lupus Erythematosus: Implications for Clinical Trial Design. Arthritis Rheumatol 69, 785-790. 10.1002/art.40013.
Koutroubakis, I. E., Kritikos, H., Mouzas, I. A., Spanoudakis, S. M., Kapsoritakis, A. N., Petinaki, E., Kouroumalis, E. A., and Manousos, O. N. (1998). Association between ulcerative colitis and systemic lupus erythematosus: report of two cases. Eur J Gastroenterol Hepatol 10, 437-439. 10.1097/00042737-199805000-00016.
Lee, S. H. (2015). Intestinal permeability regulation by tight junction: implication on inflammatory bowel diseases. Intest Res 13(1), 11-18. doi: 10.5217/ir.2015.13.1.11.
Liu, C., Finegold, S. M., Song, Y., and Lawson, P. A. (2008). Reclassification of Clostridium coccoides, Ruminococcus hansenii, Ruminococcus hydrogenotrophicus, Ruminococcus luti, Ruminococcus productus and Ruminococcus schinkii as Blautia coccoides gen. nov., comb. nov., Blautia hansenii comb. nov., Blautia hydrogenotrophica comb. nov., Blautia luti comb. nov., Blautia producta comb. nov., Blautia schinkii comb. nov. and description of Blautia wexlerae sp. nov., isolated from human faeces. Int J Syst Evol Microbiol 58, 1896-1902. 10.1099/ijs.0.65208-0.
Low, A. J., Koziol, A. G., Manninger, P. A., Blais, B., and Carrillo, C. D. (2019). ConFindr: rapid detection of intraspecies and cross-species contamination in bacterial whole-genome sequence data. Peer J7, e6995. 10.7717/peerj.6995.
Manfredo Vieira, S., Hiltensperger, M., Kumar, V., Zegarra-Ruiz, D., Dehner, C., Khan, N., et al. (2018). Translocation of a gut pathobiont drives autoimmunity in mice and humans. Science 359(6380), 1156-1161. doi: 10.1126/science.aar7201.
Marks, B. R., and Craft, J. (2009). Barrier immunity and IL-17. Semin Immunol 21, 164-171. 10.1016/j.smim.2009.03.001.
Mishra, A. K., Driessen, N. N., Appelmelk, B. J., and Besra, G. S. (2011). Lipoarabinomannan and related glycoconjugates: structure, biogenesis and role in Mycobacterium tuberculosis physiology and host-pathogen interaction. FEMS Microbiol Rev 35, 1126-1157. 10.1111/j.1574-6976.2011.00276.x.
Mishra, A. K., Klein, C., Gurcha, S. S., Alderwick, L. J., Babu, P., Hitchen, P. G., Morris, H. R., Dell, A., Besra, G. S., and Eggeling, L. (2008). Structural characterization and functional properties of a novel lipomannan variant isolated from a Corynebacterium glutamicum pimB′ mutant. Antonie Van Leeuwenhoek 94, 277-287. 10.1007/s10482-008-9243-1.
Moeller, A. H., Shilts, M., Li, Y., Rudicell, R. S., Lonsdorf, E. V., Pusey, A. E., Wilson, M. L., Hahn, B. H., and Ochman, H. (2013). SIV-induced instability of the chimpanzee gut microbiome. Cell Host Microbe 14, 340-345. 10.1016/j.chom.2013.08.005.
Moore, W. E. C., Johnson, J. L., and L. V. Holdeman (1976). Emendation of Bacteroidaceae and Butyrivibrio and Descriptions of Desulfomonas gen. nov. and Ten New Species in the Genera Desulfomonas, Butyrivibrio, Eubacterium, Clostridium, and Ruminococcus. International Journal of Systematic and Evolutionary Microbiology 26(2), 238-252.
Nava, G. M., Friedrichsen, H. J., and Stappenbeck, T. S. (2011). Spatial organization of intestinal microbiota in the mouse ascending colon. ISME J5, 627-638. 10.1038/ismej.2010.161.
Ogunrinde, E., Zhou, Z., Luo, Z., Alekseyenko, A., Li, Q. Z., Macedo, D., et al. (2019). A Link Between Plasma Microbial Translocation, Microbiome, and Autoantibody Development in First-Degree Relatives of Systemic Lupus Erythematosus Patients. Arthritis Rheumatol 71(11), 1858-1868. doi: 10.1002/art.40935.
Oksanen, J. G. B., F.; Friendly, M.; Kindt, R.; and Wagner, H. (2020). vegan community ecology package version 2.5-7 Nov. 2020.
Pedersen, H. L., Horvei, K. D., Thiyagarajan, D., Seredkina, N., and Rekvig, O. P. (2015). Murine and Human Lupus Nephritis: Pathogenic Mechanisms and Theoretical Strategies for Therapy. Semin Nephrol 35, 427-438. 10.1016/j.semnephrol.2015.08.004.
Pelzek, A. J., Gronwall, C., Rosenthal, P., Greenberg, J. D., McGeachy, M., Moreland, L., et al. (2017). Persistence of Disease-Associated Anti-Citrullinated Protein Antibody-Expressing Memory B Cells in Rheumatoid Arthritis in Clinical Remission. Arthritis Rheumatol 69(6), 1176-1186. doi: 10.1002/art.40053.
Pelzek, A. J., Shopsin, B., Radke, E. E., Tam, K., Ueberheide, B. M., Fenyo, D., et al. (2018a). Human Memory B Cells Targeting Staphylococcus aureus Exotoxins Are Prevalent with Skin and Soft Tissue Infection. MBio 9(2), pii: e02125-02117. doi: 10.1128/mBio.02125-17.
Pelzek, A. J., Shopsin, B., Radke, E. E., Tam, K., Ueberheide, B. M., Fenyo, D., et al. (2018b). Human Memory B Cells Targeting Staphylococcus aureus Exotoxins Are Prevalent with Skin and Soft Tissue Infection. mBio 9(2). doi: 10.1128/mBio.02125-17.
Png, C. W., Linden, S. K., Gilshenan, K. S., Zoetendal, E. G., McSweeney, C. S., Sly, L. I., et al. (2010). Mucolytic bacteria with increased prevalence in IBD mucosa augment in vitro utilization of mucin by other bacteria. Am J Gastroenterol 105(11), 2420-2428. doi: 10.1038/ajg.2010.281.
Qin, J., Li, R., Raes, J., Arumugam, M., Burgdorf, K. S., Manichanh, C., et al. (2010). A human gut microbial gene catalogue established by metagenomic sequencing. Nature 464(7285), 59-65. doi: 10.1038/nature08821.
Quinlan, A. R., and Hall, I. M. (2010). BEDTools: a flexible suite of utilities for comparing genomic features. Bioinformatics 26, 841-842. 10.1093/bioinformatics/btq033.
Radke, E. E., Brown, S. M., Pelzek, A. J., Fulmer, Y., Hernandez, D. N., Torres, V. J., et al. (2018). Hierarchy of human IgG recognition within the Staphylococcus aureus immunome. Sci Rep 8(1), 13296. doi: 10.1038/s41598-018-31424-3.
Rosser, E. C., and Mauri, C. (2016). A clinical update on the significance of the gut microbiota in systemic autoimmunity. J Autoimmun 74, 85-93. 10.1016/j.jaut.2016.06.009.
Round, J. L., and Mazmanian, S. K. (2009). The gut microbiota shapes intestinal immune responses during health and disease. Nat Rev Immunol 9(5), 313-323. doi: 10.1038/nri2515.
Sagheddu, V., Patrone, V., Miragoli, F., Puglisi, E., and Morelli, L. (2016). Infant Early Gut Colonization by Lachnospiraceae: High Frequency of Ruminococcus gnavus. Front Pediatr 4, 57. doi: 10.3389/fped.2016.00057.
Saad, R., Rizkallah, M. R., and Aziz, R. K. (2012). Gut Pharmacomicrobiomics: the tip of an iceberg of complex interactions between drugs and gut-associated microbes. Gut Pathog 4, 16. 10.1186/1757-4749-4-16.
Seemann, T. (2014). Prokka: rapid prokaryotic genome annotation. Bioinformatics 30, 2068-2069. 10.1093/bioinformatics/btu153.
Schloissnig, S., Arumugam, M., Sunagawa, S., Mitreva, M., Tap, J., Zhu, A., et al. (2013). Genomic variation landscape of the human gut microbiome. Nature 493(7430), 45-50. doi: 10.1038/nature11711.
Silverman, G. J. A., D. F.; Gronwall, C.; Gunnarsson, I.; Svenungsson, E. (2019). Validation of a Serologic Antibody Biomarker Against a Candidate Gut Pathobiont for the Diagnosis of Lupus Nephritis [abstract]. Arthritis Rheumatol. 71, abs 1915.
Sorbara, M. T., Littmann, E. R., Fontana, E., Moody, T. U., Kohout, C. E., Gjonbalaj, M., et al. (2020). Functional and Genomic Variation between Human-Derived Isolates of Lachnospiraceae Reveals Inter- and Intra-Species Diversity. Cell Host Microbe 28(1), 134-146 e134. doi: 10.1016/j.chom.2020.05.005.
Sturgeon, C., and Fasano, A. (2016). Zonulin, a regulator of epithelial and endothelial barrier functions, and its involvement in chronic inflammatory diseases. Tissue Barriers 4(4), e1251384. doi: 10.1080/21688370.2016.1251384.
Suarez-Fueyo, A., Bradley, S. J., and Tsokos, G. C. (2016). T cells in Systemic Lupus Erythematosus. Curr Opin Immunol 43, 32-38. 10.1016/j.coi.2016.09.001.
Tatituri, R. V. V., Illarionov, P. A., Dover, L. G., Nigou, J., Gilleron, M., Hitchen, P., Krumbach, K., Morris, H. R., Spencer, N., Dell, A., et al. (2007). Inactivation of Corynebacterium glutamicum NCgl0452 and the role of MgtA in the biosynthesis of a novel mannosylated glycolipid involved in lipomannan biosynthesis. J Biol Chem 282, 4561-4572. 10.1074/jbc.M608695200.
Vacca, M., Celano, G., Calabrese, F. M., Portincasa, P., Gobbetti, M., and De Angelis, M. (2020). The Controversial Role of Human Gut Lachnospiraceae. Microorganisms 8(4). doi: 10.3390/microorganisms8040573.
Wesemann, D. R. (2015). Microbes and B cell development. Adv Immunol 125, 155-178. doi: 10.1016/bs.ai.2014.09.005.
Wick, R. R., Judd, L. M., Gorrie, C. L., and Holt, K. E. (2017). Unicycler: Resolving bacterial genome assemblies from short and long sequencing reads. PLoS Comput Biol 13, e1005595. 10. 1371/journal.pcbi. 1005595.
Yang, C. C.-L., A.; Moran, T. W.; Cerutti, A.; Faith, J. J. (2020). Immunoglobulin A Antibody Composition Is Sculpted to Bind the Self Gut Microbiome. bioRxiv. doi: 10.1101/2020.11.30.405332.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. An isolated antibody, or antigen-binding fragment thereof, that specifically recognizes a lipoglycan-containing antigen associated with Ruminococcus gnavus strain CC55_001C/HM-1056, S107-86, S47-18, and/or S107-48.

2. The isolated antibody or antigen-binding fragment of claim 1, wherein the lipoglycan-containing antigen comprises a lipoglycan which has a molecular weight between about 20,000 Daltons and about 30,000 Daltons.

3. The isolated antibody or antigen-binding fragment of claim 1 or 2, wherein the lipoglycan-containing antigen comprises a lipoglycan selected from those listed in Table 7.

4. The isolated antibody or antigen-binding fragment of claim, wherein the lipoglycan-containing antigen comprises a lipoglycan comprising three fatty acids with acyl chain composition of 47:0, one glycerol, eight hexoses, five N-acetyl-hexosamines, and three hexuronic acids (tri-acyl lipoglycan 47:0 with a mono-isotopic mass of about 3632).

5. The isolated antibody or antigen-binding fragment of claim 3, wherein the lipoglycan-containing antigen comprises a lipoglycan comprising two fatty acids with acyl chain composition of 31:0, one glycerol, eight hexoses, five N-acetyl-hexosamines, and three hexuronic acids (di-acyl lipoglycan 31:0 with a mono-isotopic mass of about 3394).

6. The isolated antibody or antigen-binding fragment of claim 3, wherein the lipoglycan-containing antigen comprises a lipoglycan comprising one fatty acid with acyl chain composition of 16:0, one glycerol, eight hexoses, five N-acetyl-hexosamines, and three hexuronic acids (mono-acyl lipoglycan 16:0 with a mono-isotopic mass of about 3170).

7. The isolated antibody or antigen-binding fragment of claim 3, wherein the lipoglycan-containing antigen comprises a lipoglycan comprising one margaric acid with acyl chain composition of 17:0, one glycerol, eight hexoses, five N-acetyl-hexosamines, and three hexuronic acids (mono-acyl lipoglycan 17:0 with a mono-isotopic mass of about 3184).

8. The isolated antibody or antigen-binding fragment of any one of claims 1-7, wherein the lipoglycan-containing antigen is the same as the lipoglycan-containing antigen obtained using a method selected from:
(i) Method 1 comprising the steps:
a). culturing Ruminococcus guavas strain CC55_001C/HM-1056, S107-86, 547-18, or S107-48 at 37° C. under anaerobic conditions for 2-7 days, and
b). producing bacterial extract in the presence of a lysozyme, Serratia marcescens endonuclease, Proteinase K, and a detergent under non-denaturing conditions;
(ii) Method 2 comprising the steps:
a). culturing Ruminococcus gnavus strain CC55_001C/HM-1056, S107-86, S47-18, or S107-48 in rich nutrient media at 37° C. under anaerobic conditions (75% N₂, 20% CO₂, and 5% H₂) for 2-7 days,
b). pelleting bacteria by centrifugation,
c). producing a bacterial extract in a protein extraction buffer in the presence of lysozyme, Serratia marcescens endonuclease, and a detergent under non-denaturing conditions,
d). treating the extract obtained in step (c) with Proteinase K,
e). incubating the treated extract obtained in step (d) at 55° C. for about 10 minutes,
f). removing cell debris by centrifugation, and
g). using the supernatant as an antigen preparation; or
(iii) Method 3 comprising the steps:
a). disrupting Ruminococcus gnavus strain CC55-001C/HM-1056, S107-86, S47-18, or S107-48 cells with a French press,
b). ultracentrifugating to obtain a precipitate and produce an ultracentrifugation supernatant,
c). subjecting the ultracentrifugation supernatant obtained in step (b) to butanol-water extraction and isolating therefrom an aqueous phase,
d). applying the aqueous phase from step (c) to a hydrophobic interaction chromatography matrix, and
e). isolating lipoglycan-containing fractions.

9. The isolated antibody or antigen-binding fragment of any one of claims 1-8, comprising a heavy chain complementarity determining region 1 (CDR1), a heavy chain CDR2, a heavy chain CDR3 of a heavy chain variable region (VH) comprising an amino acid sequence of SEQ ID NO: 4 or 13; and/or a light chain CDR1, a light chain CDR1, a light chain CDR2, and a light chain CDR3 of a light chain variable region (VL) comprising an amino acid sequence of SEQ ID NO: 9 or 15.

10. The isolated antibody or antigen-binding fragment of any one of claims 1-9, comprising a heavy chain CDR1, a heavy chain CDR2 and a heavy chain CDR3 of a VH comprising an amino acid sequence of SEQ ID NO: 4, and a light chain CDR1, a light chain CDR2 and a light chain CDR3 of a VL comprising an amino acid sequence of SEQ ID NO: 9; or a heavy chain CDR1, a heavy chain CDR2 and a heavy chain CDR3 of a VH comprising an amino acid sequence of SEQ ID NO: 13, and a light chain CDR1, a light chain CDR2 and alight chain CDR3 of a VL comprising an amino acid sequence of SEQ ID NO: 15.

11. The isolated antibody or antigen-binding fragment of any one of claims 1-10, comprising a heavy chain CDR1 comprising the amino acid sequence of SEQ ID NO: 21, a heavy chain CDR2 comprising the amino acid sequence of SEQ ID NO: 22, and a heavy chain CDR3 comprising the amino acid sequence of SEQ ID NO: 3; or
a heavy chain CDR1 comprising the amino acid sequence of SEQ ID NO: 21, a heavy chain CDR2 comprising the amino acid sequence of SEQ ID NO: 25, and a heavy chain CDR3 comprising the amino acid sequence of SEQ ID NO: 12.

12. The isolated antibody or antigen-binding fragment of any one of claims 1-10 and 11, comprising
a light chain CDR1 comprising the amino acid sequence of SEQ ID NO: 24, a light chain CDR2 comprising the amino acid sequence of KAS, and a light chain CDR3 comprising the amino acid sequence of SEQ ID NO: 8.

13. The isolated antibody or antigen-binding fragment of any one of claims 1-10 and 11-12, comprising
the heavy chain CDR1 of SEQ ID NO: 21, the heavy chain CDR2 of SEQ ID NO: 22, the heavy chain CDR3 of SEQ ID NO: 3, the light chain CDR1 of SEQ ID NO: 24, the light chain CDR2 of KAS, and the light chain CDR3 of SEQ ID NO: 8; or
the heavy chain CDR1 of SEQ ID NO: 21, the heavy chain CDR2 of SEQ ID NO: 25, the heavy chain CDR3 of SEQ ID NO: 12, the light chain CDR1 of SEQ ID NO: 24, the light chain CDR2 of KAS, and the light chain CDR3 of SEQ ID NO: 8.

14. The isolated antibody or antigen-binding fragment of any one of claims 1-13, comprising
a VH comprising an amino acid sequence of SEQ ID NO: 4 or 13; and/or
a V_{L} comprising an amino acid sequence of SEQ ID NO: 9 or 15.

15. The isolated antibody or antigen-binding fragment of any one of claims 1-14, comprising
a VH comprising an amino acid sequence of SEQ ID NO: 4 and a VL comprising an amino acid sequence of SEQ ID NO: 9; or
a VH comprising an amino acid sequence of SEQ ID NO: 13 and a VL comprising an amino acid sequence of SEQ ID NO: 15.

16. The isolated antibody or antigen-binding fragment of any one of claims 1-15, wherein the antibody or antigen-binding fragment is recombinant.

17. The isolated antibody or antigen-binding fragment of any one of claims 1-16, wherein the antibody or antigen-binding fragment is a human antibody, a humanized antibody, a chimeric antibody, a murine antibody, a monoclonal antibody, a single chain antibody, a Fab, a Fab′, a F(ab′)2, a Fv, a scFv, or a nanobody.

18. The isolated antibody or antigen-binding fragment of any one of claims 1-17, wherein the antibody or antigen-binding fragment is an IgG antibody.

19. The isolated antibody or antigen-binding fragment of any one of claims 1-18, wherein the antibody or antigen-binding fragment is of IgG1, IgG2, IgG3, or IgG4 subclass.

20. The isolated antibody or antigen-binding fragment of any one of claim 1-19, wherein the antibody or antigen-binding fragment is of IgG1 subclass.

21. The isolated antibody or antigen-binding fragment of any one of claim 1-19, wherein the antibody or antigen-binding fragment is of IgG2 subclass.

22. An isolated polynucleotide encoding the isolated antibody or antigen-binding fragment of any one of claims 1-21.

23. The isolated polynucleotide of claim 22, comprising
a VH-encoding nucleotide sequence of SEQ ID NO: 5 or 14; and/or
a VL-encoding nucleotide sequence of SEQ ID NO: 10 or 16.

24. The isolated polynucleotide of claim 23, comprising
a VH-encoding nucleotide sequence of SEQ ID NO: 5 and a VL-encoding nucleotide sequence of SEQ ID NO: 10; or
a VH-encoding nucleotide sequence of SEQ ID NO: 14 and a VL-encoding nucleotide sequence of SEQ ID NO: 16.

25. The isolated polynucleotide of any one of claims 22-24, wherein the polynucleotide is a DNA molecule or a derivative thereof.

26. The isolated polynucleotide of any one of claims 22-24, wherein the polynucleotide is an RNA molecule or a derivative thereof.

27. The isolated polynucleotide of claim 26, wherein the polynucleotide is an mRNA.

28. A vector comprising the polynucleotide of any one of claims 22-27.

29. The vector of claim 28, wherein the sequence encoding the isolated antibody or antigen-binding fragment is operably linked to a promoter, wherein the promoter mediates expression of the isolated antibody or antigen-binding fragment.

30. The vector of claim 28 or 29, wherein the vector is a viral vector.

31. The vector of claim 30, wherein the vector has tropism to the gastrointestinal tract.

32. A recombinant cell comprising the polynucleotide of any one of claims 22-27 or the vector of any one of claims 28-31.

33. The cell of claim 32, wherein the cell is a hybridoma.

34. The cell of claim 32 or 33, wherein the antibody or antigen-binding fragment is recombinantly produced.

35. An antibody-drug conjugate comprising the isolated antibody or antigen-binding fragment of any one of claims 1-21 conjugated to a second moiety.

36. The antibody-drug conjugate of claim 35, wherein the second moiety is a bacterial toxin or an antibiotic.

37. A pharmaceutical composition comprising the isolated antibody or antigen-binding fragment of any one of claims 1-21, or the polynucleotide of any one of claims 22-27, or the vector of any one of claims 28-31, or the antibody-drug conjugate of any one of claims 35-36 and a pharmaceutically acceptable excipient or carrier.

38. The pharmaceutical composition of claim 37, which is milk, yogurt, infant formula, or other dairy product.

39. The pharmaceutical composition of claim 37, wherein the pharmaceutical composition is formulated such that it is released primarily in the gastrointestinal tract.

40. The pharmaceutical composition of claim 39, wherein the pharmaceutical composition is formulated such that it is released primarily in the small intestine.

41. The pharmaceutical composition of claim 40, wherein the pharmaceutical composition is formulated such that it is released primarily in the ileum.

42. A vaccine composition comprising the polynucleotide of any one of claims 22-27 or the vector of any one of claims 28-31.

43. The vaccine composition of claim 42, wherein the polynucleotide is an mRNA.

44. A kit comprising (i) the isolated antibody or antigen-binding fragment of any one of claims 1-21, or the polynucleotide of any one of claims 22-27, or the vector of any one of claims 28-31, or the antibody-drug conjugate of any one of claims 35-36 and (ii) packaging and/or instructions for the same.

45. A method of producing the isolated antibody or antigen-binding fragment of any one of claims 1-21, wherein said method comprises culturing the recombinant cell of any one of claims 32-34, and isolating said antibody or antigen-binding fragment.

46. A method of producing the isolated antibody or antigen-binding fragment of any one of claims 1-21, wherein said method comprises isolating said antibody or antigen-binding fragment from an animal immunized with a lipoglycan-containing antigen associated with Ruminococcus gnavus strain CC55_001C/HM-1056, S107-86, 547-18, and/or S107-48 or a Ruminococcus gnavus strain that produces a lipoglycan that is structurally or functionally equivalent to Ruminococcus gnavus strain CC55_001C/HM-1056, S107-86, S47-18, and/or S107-48, or colonized with Ruminococcus gnavus strain CC55_001C/HM-1056, S107-86, S47-18, and/or S107-48 or a Ruminococcus gnavus strain that produces a lipoglycan that is structurally or functionally equivalent to Ruminococcus gnavus strain CC55_001C/HM-1056, S107-86, S47-18, and/or S107-48 in the gastrointestinal tract, or genetically modified to produce said antibody or antigen-binding fragment.

47. The method of claim 46, wherein the animal is genetically modified with the polynucleotide of any one of claims 22-27 or the vector of any one of claims 28-31.

48. The method of claim 47, wherein the animal is a dairy animal and said antibody or antigen-binding fragment is isolated from milk produced by the dairy animal.

49. The method of claim 48, wherein the dairy animal is a goat, a cow, a buffalo, a sheep, or a camel.

50. A method of reducing the amount of a lipoglycan-containing antigen and/or of a bacterial strain associated with the lipoglycan-containing antigen in a subject in need thereof, comprising administering to the subject an effective amount of the isolated antibody or antigen-binding fragment of any one of claims 1-21, or the polynucleotide of any one of claims 22-27, or the vector of any one of claims 28-31, or the antibody-drug conjugate of any one of claims 35-36, or the pharmaceutical composition of any one of claims 37-41.

51. A method of treating or preventing a disease or disorder caused by a lipoglycan-containing antigen and/or a bacterial strain associated with the lipoglycan-containing antigen in a subject in need thereof, comprising administering to the subject an effective amount of the isolated antibody or antigen-binding fragment of any one of claims 1-21, or the polynucleotide of any one of claims 22-27, or the vector of any one of claims 28-31, or the antibody-drug conjugate of any one of claims 35-36, or the pharmaceutical composition of any one of claims 37-41, or the vaccine of any one of claims 42-43.

52. The method of any one of claims 46-51, wherein the lipoglycan-containing antigen comprises a lipoglycan which has a molecular weight between about 20,000 Daltons to about 30,000 Daltons.

53. The method of any one of claims 46-52, wherein the lipoglycan-containing antigen comprises a lipoglycan which is a lipoglycan selected from those listed in Table 7.

54. The method of claim 53, wherein the lipoglycan-containing antigen comprises a lipoglycan comprising three fatty acids with acyl chain composition of 47:0, one glycerol, eight hexoses, five N-acetyl-hexosamines, and three hexuronic acids (tri-acyl lipoglycan 47:0 with a mono-isotopic mass of about 3632).

55. The method of claim 53, wherein the lipoglycan-containing antigen comprises a lipoglycan comprising two fatty acids with acyl chain composition of 31:0, one glycerol, eight hexoses, five N-acetyl-hexosamines, and three hexuronic acids (di-acyl lipoglycan 31:0 with a mono-isotopic mass of about 3394).

56. The method of claim 53, wherein the lipoglycan-containing antigen comprises a lipoglycan comprising one fatty acid with acyl chain composition of 16:0, one glycerol, eight hexoses, five N-acetyl-hexosamines, and three hexuronic acids (mono-acyl lipoglycan 16:0 with a mono-isotopic mass of about 3170).

57. The method of claim 53, wherein the lipoglycan-containing antigen comprises a lipoglycan comprising a lipoglycan comprising one margaric acid with acyl chain composition of 17:0, one glycerol, eight hexoses, five N-acetyl-hexosamines, and three hexuronic acids (mono-acyl lipoglycan 17:0 with a mono-isotopic mass of about 3184).

58. The method of any one of claims 46-57, wherein the lipoglycan-containing antigen is the same as the lipoglycan-containing antigen obtained using a method selected from:
(i) Method 1 comprising the steps:
a). culturing Ruminococcus gnavus strain CC55_001C/HM-1056, S107-86, S47-18, or S107-48 at 37° C. under anaerobic conditions for 2-7 days, and
b). producing bacterial extract in the presence of a lysozyme, Serratia marcescens endonuclease, Proteinase K, and a detergent under non-denaturing conditions;
(ii) Method 2 comprising the steps:
a). culturing Ruminococcus gnavus strain CC55_001C/1HM-1056, S107-86, 547-18, or S107-48 in rich nutrient media at 37° C. under anaerobic conditions (75% N₂, 20% CO₂, and 5% H₂) for 2-7 days,
b). pelleting bacteria by centrifugation,
c). producing a bacterial extract in a protein extraction buffer in the presence of lysozyme, Serratia marcescens endonuclease, and a detergent under non-denaturing conditions,
d). treating the extract obtained in step (c) with Proteinase K,
e). incubating the treated extract obtained in step (d) at 55° C. for about 10 minutes,
f). removing cell debris by centrifugation, and
g). using the supernatant as an antigen preparation; or
(iii) Method 3 comprising the steps:
a). disrupting Ruminococcus gnavus strain CC55_001C/HM-1056, S107-86, S47-18, or S107-48 cells with a French press,
b). ultracentrifugating to obtain a precipitate and produce an ultracentrifugation supernatant,
c). subjecting the ultracentrifugation supernatant obtained in step (b) to butanol-water extraction and isolating therefrom an aqueous phase,
d). applying the aqueous phase from step (c) to a hydrophobic interaction chromatography matrix, and
e). isolating lipoglycan-containing fractions.

59. The method of any one of claims 50-58, wherein the bacterial strain is a strain of Ruminococcus gnavus.

60. The method of claim 59, wherein the lipoglycan-associated bacterial strain is Ruminococcus gnavus strain CC55_001C/HM-1056, S107-86, S47-18, or S107-48.

61. The method of any one of claims 51-60, wherein the disease or disorder is systemic lupus erythematosus (SLE), lupus nephritis, hidradenitis suppurativa, inflammatory bowel disease (IBD), incomplete lupus (ILE), undifferentiated connective tissue disease (UCTD), complications of SLE, IgA nephropathy, Henoch Schonlein Purpura (HSP), and other types of glomerulonephritis.

62. The method of claim 61, wherein the lupus nephritis is proliferative lupus nephritis, membranous lupus nephritis, membranoproliferative lupus nephritis, or mesangial glomerulonephritis.

63. The method of any one of claims 50-61, wherein the isolated antibody or antigen-binding fragment, or the polynucleotide, or the vector, or the pharmaceutical composition of any one of claims 32-34 is administered by a route selected from oral, nasal, rectal, mucosal, sublingual, and via naso/oro-gastric gavage.

64. The method of any one of claims 50-63, wherein the subject is human.


SEQUENCE LISTING (25 sequences)

SEQ ID NO: 1 - protein, 10 aa, synthetic construct
GDSITSGYWN

SEQ ID NO: 2 - protein, 15 aa, synthetic construct
INYSGSTYYNPSLRS

SEQ ID NO: 3 - protein, 14 aa, synthetic construct
ARYYYHDDSYAMDN

SEQ ID NO: 4 - protein, 120 aa, synthetic construct
EVQLQESGPSLVKPSQTLSLTCSVTGDSITSGYWNWIRKFPGNKLEYLGYINYSGSTYYNPSLRSRISITRDTSKNQYYLQLNAVTTEDTATYYCARYYYHDDSYAMDNWGQGTSVTVSS

SEQ ID NO: 5 - DNA, 360 bp, synthetic construct
  source 1..360: mol_type = other DNA
gaggtgcagcttcaggagtcaggacctagcctcgtgaaaccttctcagactctgtccctcacctgttctgtcactggcgactccatcaccagtggttactggaactggatccggaaattcccagggaataaacttgagtatttggggtacataaactatagtggtagcacttactacaatccatctctcagaagtcgaatctccatcactcgagacacatccaagaaccagtactacctgcagttgaatgctgtgactactgaggacacagccacatattactgtgcaagatattattatcacgatgatagctacgctatggacaactggggtcaaggaacctcagtcaccgtctcctca

SEQ ID NO: 6 - protein, 9 aa, synthetic construct
NINVWLSWF

SEQ ID NO: 7 - protein, 8 aa, synthetic construct
NLHTGVPS

SEQ ID NO: 8 - protein, 9 aa, synthetic construct
QQGQSYPLT

SEQ ID NO: 9 - protein, 107 aa, synthetic construct
DIQMNQSPSSLSASLGDTITITCHASQNINVWLSWFQQKPGNIPKLLIYKASNLHTGVPSRFSGTGSGTGFTLTISSLQPEDIATYYCQQGQSYPLTFGAGTKLELK

SEQ ID NO: 10 - DNA, 321 bp, synthetic construct
  source 1..321: mol_type = other DNA
gacatccagatgaaccagtctccatccagtctgtctgcatcccttggagacacaattaccatcacttgccatgccagtcagaacattaatgtttggttaagctggttccagcagaaaccaggaaatattcctaaactattgatctataaggcttccaacttgcacacaggcgtcccatcaaggtttagtggcactggatctggaacaggtttcacattaaccatcagcagcctgcagcctgaagacattgccacttactactgtcaacagggtcaaagttatcctctcacgttcggtgctgggaccaagctggagctgaaa

SEQ ID NO: 11 - protein, 15 aa, synthetic construct
INYSGTTYYNPSLKS

SEQ ID NO: 12 - protein, 14 aa, synthetic construct
ARYYYYDSSYAMDY

SEQ ID NO: 13 - protein, 120 aa, synthetic construct
EVQLQESGPSLVKPSQTLSLTCSVTGDSITSGYWNWIRKFPGNKLEYMGYINYSGTTYYNPSLKSRISITRDTSKNQYYLQLNSVTTEDTATYYCARYYYYDSSYAMDYWGQGTSVTVSS

SEQ ID NO: 14 - DNA, 360 bp, synthetic construct
  source 1..360: mol_type = other DNA
gaggtgcagcttcaggagtcaggacctagcctcgtgaaaccttctcagactctgtccctcacctgttctgtcactggcgactccatcaccagtggttactggaactggatccggaaattcccagggaataaacttgaatacatgggatacataaattacagtggtaccacttactacaatccatctctcaaaagtcgaatctccatcactcgagacacatccaagaaccagtactacctacagttgaattctgtgactactgaggacacagccacatattactgtgcaagatattattactacgatagtagctacgctatggactactggggtcaaggaacctcagtcaccgtctcctca

SEQ ID NO: 15 - protein, 107 aa, synthetic construct
DIQMNQSPSSLSASLGDTITITCHASQNINVWLSWFQQKPGNIPKLLIYKASNLHTGVPSRFSGSGSGTGFTLTISSLQPEDIATYYCQQGQSYPLTFGAGTKLELK

SEQ ID NO: 16 - DNA, 321 bp, synthetic construct
  source 1..321: mol_type = other DNA
gacatccagatgaaccagtctccatccagtctgtctgcatcccttggagacacaattaccatcacttgccatgccagtcagaacattaatgtttggttaagctggttccagcagaaaccaggaaatattcctaaactgttgatctataaggcttccaacttgcacacaggcgtcccatcaaggtttagtggcagtggctctggaacaggtttcacattaaccatcagcagcctgcagcctgaagacattgccacttactactgtcaacagggtcaaagttatcctctcacgttcggtgctgggaccaagctggagctgaaa

SEQ ID NO: 17 - DNA, 21 bp, synthetic construct
  source 1..21: mol_type = other DNA
actcctacgggaggcagcagt

SEQ ID NO: 18 - DNA, 18 bp, synthetic construct
  source 1..18: mol_type = other DNA
attaccgcggctgctggc

SEQ ID NO: 19 - DNA, 22 bp, synthetic construct
  source 1..22: mol_type = other DNA
ggactgcatttggaactgtcag

SEQ ID NO: 20 - DNA, 23 bp, synthetic construct
  source 1..23: mol_type = other DNA
aacgtcagtcatcgtccagaaag

SEQ ID NO: 21 - protein, 8 aa, synthetic construct
GDSITSGY

SEQ ID NO: 22 - protein, 7 aa, synthetic construct
INYSGST

SEQ ID NO: 23 - protein, 14 aa, synthetic construct
ARYYYHDDSYAMDN

SEQ ID NO: 24 - protein, 6 aa, synthetic construct
QNINVW

SEQ ID NO: 25 - protein, 7 aa, synthetic construct
INYSGTT